SEQUENCE LISTING (42 sequences)

SEQ ID NO: 1 - DNA, 978 bp, Azospirillum oryzae
atgattgatcagcgtaccagcgactttctaagcgagttcctggcgtcaagccaccgtgatccggcgcgtttagatagcttcctgcttcacgggccaggccgcggcgctcgcgcggcgaaaccgcgtctgaaaattgcgttttttgacttttggccggaatttgacccggctgcgaacttttttgtggacattctgagcgcgcgctttgatgtaagcgttgtggataacgatagcgacttagcgattgtgagcgtgtttggcacccgccatcgcgaagcgcgtaccgcgcgtagcatgttttttaccggcgaaaatgtgcgtccgccgttagatggcgtggatatgagcgtgagctttgatcgcattgatgacccacgccactatcgcctgccgttatatgtgatgcacgcgtgggatcataggcgtgaaggggcgacgccgcacttttgccagagcgtgctgccgccggtgccgccgacgcgtgaagaagcggcgaaacgtaagttttgcgcgtttttatataaaaatccaaactgcgcgaggcgcaacgactttttccagatgttatgcgcgaggcgccacgtggaaagcgtgggctggctgctgaataataccggcagcgtggtgaaaatgggctggctgccgaaaattcgtgtgttcagccgctaccgctttgcgtttgcgtttgaaaatgcgagccacccaggctacctgacggagaaaattttagatgcgtttcaagcgggcgcggtgccgttatattggggcgacccaggagtgctgcgcgacgttgcggccggcagctttattgatgtgagccgctacgcgagcgatgaagaagcgtgcgacgcgattctggccgcggatgatgattacgatacctaccgccgctaccgcagcacgccgccgtttctgggcgcggaagatttttactttgatgcgtttcggctggccgagtggattgagagccgtctataa

SEQ ID NO: 2 - protein, 325 aa, Azospirillum oryzae
MIDQRTSDFLSEFLASSHRDPARLDSFLLHGPGRGARAAKPRLKIAFFDFWPEFDPAANFFVDILSARFDVSVVDNDSDLAIVSVFGTRHREARTARSMFFTGENVRPPLDGVDMSVSFDRIDDPRHYRLPLYVMHAWDHRREGATPHFCQSVLPPVPPTREEAAKRKFCAFLYKNPNCARRNDFFQMLCARRHVESVGWLLNNTGSVVKMGWLPKIRVFSRYRFAFAFENASHPGYLTEKILDAFQAGAVPLYWGDPGVLRDVAAGSFIDVSRYASDEEACDAILAADDDYDTYRRYRSTPPFLGAEDFYFDAFRLAEWIESRL

SEQ ID NO: 3 - DNA, 978 bp, Azospirillum lipoferum
atgattgatcagcgtacgagcgattttctgagcgagtttctagcgtcaccaaaccgtgatccagcggtgttagatcggtttctgcttcatgggccggagcgcgggggccgtgcggccagaccgcgcctgaaaattgcgttttttgacttttggccggaatttgacccgagcgcgaatttttttgtagaaattctgagcagccgctttgatgtgagcgtggttgataatgatagcgatttagcgattctgagcgtgtttggcgaacgccaccgcgaagcgcgtaccgcgcgcgcgctgttttttaccggcgaaaatgtgcgcccgccgttagatggcgtggatatgagcgtgagctttgatcgcattgatcatccacgtcattatcgcctgccgttatacgtgatgcatgcgtgggatcaccgtcgcgaaggggcgaccccgcatttttgccatccggtgctgccgccggtgccgccgacgcgtgaagaagcggcgaaacgtaagttttgcgcgtttttatataaaaatcctcactgcgcgcgccgcaacgatttttttcagatgctgtgcgcgcggcgccatgtggaaagcgtgggctggctgctgaataataccggcagcgtggtgaaaatgggctggctgccgaaaattcgcgtgtttgcgcgctatcgctttgcgtttgcgtttgaaaacgcggcgcatccaggctatctgaccgagaaaattctggatgcgtttcaggccgggacggtaccgttatactggggcgacagcggcgtgctgcgcgacgttgcggccggcagctttattgatgtgagccgctatgcgagcgatgaagaagcgattgaagcgattctggcgattgatgatgactatgatagctatcgccggtaccgcggcacggcgccatttttaggcaccgaggacttttactttgacgcgtaccggctggccgagtggattgagagccgcctgtag

SEQ ID NO: 4 - protein, 325 aa, Azospirillum lipoferum
MIDQRTSDFLSEFLASPNRDPAVLDRFLLHGPERGGRAARPRLKIAFFDFWPEFDPSANFFVEILSSRFDVSVVDNDSDLAILSVFGERHREARTARALFFTGENVRPPLDGVDMSVSFDRIDHPRHYRLPLYVMHAWDHRREGATPHFCHPVLPPVPPTREEAAKRKFCAFLYKNPHCARRNDFFQMLCARRHVESVGWLLNNTGSVVKMGWLPKIRVFARYRFAFAFENAAHPGYLTEKILDAFQAGTVPLYWGDSGVLRDVAAGSFIDVSRYASDEEAIEAILAIDDDYDSYRRYRGTAPFLGTEDFYFDAYRLAEWIESRL

SEQ ID NO: 5 - DNA, 990 bp, Basilea psittacipulmonis
atgtcagtgctgaaaaaactggtaagaacgctgaaaaagaaaaaagatattcccagcgaaaatcaggaagatattaagccgcaggaatttggacatatcaaacattatcatttctggcctctgtcgaacgaaacgttttttaaccaatttgcgcaggaaaaaaaccttgatctgagccagacggccctaattagctgctttggggaactatcagcgattcctaaaatccctgaacggtataaagtgttttttacgggggaaaatatctatcatccggatcgcatctcatattcggatccggagctctatcgcatggtggatctgtatttaggctttgaataccggacggaaccgaagtatctacgctttccgctgtgggtttggtatttatgcggcctgacgaaaaaaccgcatttttcacatgaatcaattgcggaatttattcggaaaatgaaccaacctgagtttcgcctgcagtcatcaaggaatcgcttttgcagccatattagcagccatgatacgaacggcattcggaaacggatgattgatttaattttaccgattgcgtcagtggattgcgccggcaagtttatgaacaacacggatgagttaaaagcgaagtttaatgatgacaagatcgactatctaaaacagtatcggtttaatctctgtcctgaaaactcagaatcagtcgggtatatcacggaaaagatttttgaatcaattatggccgggtgcattccaatttattggggaggagtcaagcagctttttgtcgaaccggatattttaaatccggaagcatttatttactacgaaaaagggaaagaagagcaattagcgaaacaggttgaagaactttggatatcacctaaacggtatgaagagtttgcagcaattgccccgtttaaagaggacgcagccgaagtgatttatacgtggattgaagaactggaaaaacggctacgggcatttgaaccaaaagcctaa

SEQ ID NO: 6 - protein, 329 aa, Basilea psittacipulmonis
MSVLKKLVRTLKKKKDIPSENQEDIKPQEFGHIKHYHFWPLSNETFFNQFAQEKNLDLSQTALISCFGELSAIPKIPERYKVFFTGENIYHPDRISYSDPELYRMVDLYLGFEYRTEPKYLRFPLWVWYLCGLTKKPHFSHESIAEFIRKMNQPEFRLQSSRNRFCSHISSHDTNGIRKRMIDLILPIASVDCAGKFMNNTDELKAKFNDDKIDYLKQYRFNLCPENSESVGYITEKIFESIMAGCIPIYWGGVKQLFVEPDILNPEAFIYYEKGKEEQLAKQVEELWISPKRYEEFAAIAPFKEDAAEVIYTWIEELEKRLRAFEPKA

SEQ ID NO: 7 - DNA, 930 bp, Planctopirus limnophila
atgcaaaaaactttttctctacgtccagtcccagtggatgcaattcgatggaactttgcaaacttttggtcgggtttcgatgcattagcattcgagcggcacctgttaggtgtctctgggaaacataagttccggattagtgaacaaaaccctcagattgtgttcgaatcggtctttgggactccagggaaggggcgcgagcgatggccaaaggcacgacaggtgtggtatacgggagaaaacgtcgcaccaccactggatcagtttgataaatgtttatcgttccatcgggatattaaggatccacggcatttgcgttggccatactatctactgcacttagcaagcttaccaatgtctttcaatgatttggtgaagtgccagagctcagtgtcgacttgggcagaacgtccagggttctgtgcatttattgcatttaacgagggatgccagacgcggaaccggtttgtggaaaagctaagtcgataccgtcgagtggattgtccaggtcgggtcttaaataatatgacgagtgagacactgggtcagcgagggaacttgcatgggaaaattaacttcctgaagcaatataaatacgcagtgtgcttcgagaatactagcacgcgaggatcagaagggtatgtgacggaaaagctggttgacgcgatgctggcaggctgcataccattatactggggcgaccaccgggttggggaagattttaacgagaactcgttcattaacttaggagtatacgggaacgatgtgaatgcaatggtccagcatgtgattgaactggattctgacgaacggttgcaaaataacctgtttcaagagccatggttgccagaaattaagtcgtcagagcacttctctttcgaaacgagcaaggatgcaattctgaagttagtggcaaacgtaaataaatga

SEQ ID NO: 8 - protein, 309 aa, Planctopirus limnophila
MQKTFSLRPVPVDAIRWNFANFWSGFDALAFERHLLGVSGKHKFRISEQNPQIVFESVFGTPGKGRERWPKARQVWYTGENVAPPLDQFDKCLSFHRDIKDPRHLRWPYYLLHLASLPMSFNDLVKCQSSVSTWAERPGFCAFIAFNEGCQTRNRFVEKLSRYRRVDCPGRVLNNMTSETLGQRGNLHGKINFLKQYKYAVCFENTSTRGSEGYVTEKLVDAMLAGCIPLYWGDHRVGEDFNENSFINLGVYGNDVNAMVQHVIELDSDERLQNNLFQEPWLPEIKSSEHFSFETSKDAILKLVANVNK

SEQ ID NO: 9 - DNA, 951 bp, Pedobacter glucosidilyticus
atgaagtattttctgctgctggctaaacgccacaagaaatacctgaaagagaagaaaatcttccgcaactcgactatctctttttacaacttctgggaaatcgaggattacaacaacttttggctgcagaaattcatcgtagaccgtaacctgaacccaaaaaacaaatccatcaacttcttttctgtgttcggcccgcgctatgtccttaaaaagcaaaaagcagcgatcaatattttcttctcgggcgaaaccatgagccgtttcaaaaaataccacgactattgtctgcctgaagttgatctggcgctgggtttcgacgatctgcaacacgagaagtacttccgtctgccgctgtggatcctggactttttcgaaccgactgttgaccttgaaaaagctaaagaaaaactgaaacagctgaactactacaaaaacaataaaccgatcgtgcgtgaaaagttctgctctctgatcgcccgtcacgacgaaaacggcatccgtaaaaagattgtgaacacgctgaacccaatcgaaacggttgactgtgcaggcaaactgttcaacaatactgctcgcttacagaccgaattcgcgaacaacaaagtaaaatttctggagaactacaagtttaacatctgcccggaaaacaccaaccaggaatcctacaccaccgaaaaacttttcgaaagcttcgctgcaggctgtatcccgatctactggggttctgctcagaaaccggaaccgaacatcttcaaaccgtctagcatcatctttttcgatgagttcaaaaacaccctgtctgaggatgttgaacgtctacataaagatccgaaactgtacctggactttattagccagaacccgtttcaggacacagctgctgaatacatcatccagactatctccaacctggaactcaaactgaaagagatcattaaccaggcataa

SEQ ID NO: 10 - protein, 316 aa, Pedobacter glucosidilyticus
MKYFLLLAKRHKKYLKEKKIFRNSTISFYNFWEIEDYNNFWLQKFIVDRNLNPKNKSINFFSVFGPRYVLKKQKAAINIFFSGETMSRFKKYHDYCLPEVDLALGFDDLQHEKYFRLPLWILDFFEPTVDLEKAKEKLKQLNYYKNNKPIVREKFCSLIARHDENGIRKKIVNTLNPIETVDCAGKLFNNTARLQTEFANNKVKFLENYKFNICPENTNQESYTTEKLFESFAAGCIPIYWGSAQKPEPNIFKPSSIIFFDEFKNTLSEDVERLHKDPKLYLDFISQNPFQDTAAEYIIQTISNLELKLKEIINQA

SEQ ID NO: 11 - DNA, 1029 bp, Porphyromonas catoniae
atgccgatatatgatataaaagccatgaataccccgtcgaagcaaccattacgagaacgactgcatatgatgcgtcgacgtaatcgtgtgcggaagcgttcggttatagccctgattaagtcgcatttagatagctcacgttatcaggattataactggtgggattctcatgcgtctaccttctggttaccacggtttatagatttgcatctggaaccgaagaagaaaattaatttattttcgtgctttcaaaatccgttaatgctgattcgttattataaaggtgttaaaatttttttatcaggtgaaaacctgaccaataacgaacatttcggttttcacccgcgtatgctggatcaccgaataaatgaagttgatctagcgttaggtttcgaatttcgtaaagacccgaaatattaccgttttccgttatggatttaccagaatgaatttattagcccgtctgccagcttagaggacatatgtgttctggtaggccagataaacgacccgtcgacccgtcgtagcgccaagcgttctcgttttattggtcagatttcgagccatgataagggtggcatgcggggacggctgatagatctgttatctccgattgggcaaattgattgcgccggtaaatttcgtcataataccgacgaactgttagaagtgtatggtgacgataaattcaaatatttagccaactatcgttttaacttatgcccagaaaattcattaggtgagggctatattaccgaaaaagtgtttgatagcatacgtgccggttgtattccgatttactggggcgcctatttagaaccaggtattttaaacccgaaagcgattttacgttttgaagagggcaaagagcaagaattttataaccgggttaaagaattatgggaaaacgaggaagcgtatgaacagttcattctggaaccaccgtttgtcgaaggggccgccgaacgtatttgggaaattttgcagggtttacgtgaacgtttagcgccattagttgaagaagggtaa

SEQ ID NO: 12 - protein, 342 aa, Porphyromonas catoniae
MPIYDIKAMNTPSKQPLRERLHMMRRRNRVRKRSVIALIKSHLDSSRYQDYNWWDSHASTFWLPRFIDLHLEPKKKINLFSCFQNPLMLIRYYKGVKIFLSGENLTNNEHFGFHPRMLDHRINEVDLALGFEFRKDPKYYRFPLWIYQNEFISPSASLEDICVLVGQINDPSTRRSAKRSRFIGQISSHDKGGMRGRLIDLLSPIGQIDCAGKFRHNTDELLEVYGDDKFKYLANYRFNLCPENSLGEGYITEKVFDSIRAGCIPIYWGAYLEPGILNPKAILRFEEGKEQEFYNRVKELWENEEAYEQFILEPPFVEGAAERIWEILQGLRERLAPLVEEG

SEQ ID NO: 13 - DNA, 972 bp, Porphyromonas sp.
atgaacgcggtcgagcgagtacggaatatacttaattattgtattaacgaagtccaaatgtaccggcagtgtcccaattcaaaatactataatttctggccctgtgattataataataattggtttaaccatttcgtagaacaccgaggcttagctaaagaacggcaccggcttaactttttctcggtctttggtaaccctctactgccgcggattataccggggaagaaagtgttcttcactggggagaatcttgcagataactcaatacactcaatagggcgagctttcaaaaagacctttccggtatatgatctggtacttgggttcgactatgaagtagaggatagccgggtgaattatatgcggtttccattatggatagccttcctgatagatccgaccgccgattatcagaaaataaaggaaacgattgaacggattaacgacccgtcaacgcggcttaacgcgagccgggatcgtttcgcctgccttgttgccagccacgataaaactggtatacggcagaaattatatgatgtccttatgccgatagcgtcagtaacttgcccaggacggttccagaataatacgaacgagcttcacgatttatatgcaaacgacaagcgggaatatttaaaactgtttaaatttaacgtatgtccagaaaattcatcgactccgggttatataactgaaaagttattcgattcgttcgcatcaggttgtattcccatatacttcggtgggggaactgaggaaatagagcccgatattgtaaaccaaggagcgttcatacggtactgggatgatgggcgaatggactggatggacacggtacgggaactttgggaatcgccgtcagcataccgggccgttgccgagataccaccgttcaaagaacaagcagcagatgtaatttatgcctatatggaaaaccttcacgacaaacttgcggcaatagtgcggtga

SEQ ID NO: 14 - protein, 323 aa, Porphyromonas sp.
MNAVERVRNILNYCINEVQMYRQCPNSKYYNFWPCDYNNNWFNHFVEHRGLAKERHRLNFFSVFGNPLLPRIIPGKKVFFTGENLADNSIHSIGRAFKKTFPVYDLVLGFDYEVEDSRVNYMRFPLWIAFLIDPTADYQKIKETIERINDPSTRLNASRDRFACLVASHDKTGIRQKLYDVLMPIASVTCPGRFQNNTNELHDLYANDKREYLKLFKFNVCPENSSTPGYITEKLFDSFASGCIPIYFGGGTEEIEPDIVNQGAFIRYWDDGRMDWMDTVRELWESPSAYRAVAEIPPFKEQAADVIYAYMENLHDKLAAIVR

SEQ ID NO: 15 - DNA, 951 bp, Selenomonas infelix
atgttaatgcgtgcactcagaaaaatgaagcgatggggacgtgtggcgtttgattacacgaatactacgaaggatggagctgtttgctatcataattggtggccgtgtaattatgaggaagagtggtttcatcgttttgttgtacaaaatattggaacagaacgttgctatcatttcttttctgtatttggtccacgtattgcgttgacgctgccaacaccgaataaagtttttttctgtggtgaaaatgtgcataacgcagagtggccctataaaagctatcaagatcatgcacttggagatgtcaagctggctctcggatatgatgatatacaggatgaacgatatattcgatttcctctgtggttgctctatatgttcgatcctgttgttgaccgatatgccatccgtgagcgaattgaagaaatcaatcatgcagagaatacaagaaaatatgaatgtgtattgatttccagacacgataagtggaatatgcgtggtccaatttatgatgcattgaaagatcatttggctatttcctgtgctgggaaatggaagcaaaacactgatgaactgtggacggtttacaatgatgataaaccacgctatctaaaagagtttaagtttaatatctgcccggagaattttgacacgccgtattatgttacagagaagctgtttgaagcctttcggagtggaacaattcctatttatgcaggcggaggcgatcatccggagccggaaattgtgaatcgaagcgcactactcctttgggagcgaggacaaagtgatcatagtgccttggtacaggaagttatacggctcgcacgcgatgagatatactatgataaatttgtacatcaggttcgtttgcttccgtatacggaagagtttgtttatgaacagttttcatcgctgaaagagcggttgttgcagataagacgagggtga

SEQ ID NO: 16 - protein, 316 aa, Selenomonas infelix
MLMRALRKMKRWGRVAFDYTNTTKDGAVCYHNWWPCNYEEEWFHRFVVQNIGTERCYHFFSVFGPRIALTLPTPNKVFFCGENVHNAEWPYKSYQDHALGDVKLALGYDDIQDERYIRFPLWLLYMFDPVVDRYAIRERIEEINHAENTRKYECVLISRHDKWNMRGPIYDALKDHLAISCAGKWKQNTDELWTVYNDDKPRYLKEFKFNICPENFDTPYYVTEKLFEAFRSGTIPIYAGGGDHPEPEIVNRSALLLWERGQSDHSALVQEVIRLARDEIYYDKFVHQVRLLPYTEEFVYEQFSSLKERLLQIRRG

SEQ ID NO: 17 - DNA, 1437 bp, Helicobacter pylori
atgtttcagccgctgctggatgcatatgttgaaagcgcaagcattgaaaaaatggcaagcaaaagtccgcctccgctgaaaattgcagttgcaaattggtggggtgatgaagaaatcaaagagtttaaaaacagcgtcctgtactttattctgagccagcgttataccattaccctgcatcagaatccgaatgaattttccgatctggtttttggtaatccgctgggtagcgcacgtaaaattctgagctatcagaatgcaaaacgcgtgttttataccggtgaaaatgaaagcccgaacttcaacctgtttgattatgccattggtttcgatgagctggattttaatgatcgttatctgcgtatgccgctgtattatgatcgtctgcatcataaagcagaaagcgttaatgataccaccgcaccgtataaactgaaagataatagcctgtacgcactgaaaaaaccgagccattgctttaaagaaaaacatccgaatctgtgtgccgtggttaatgatgaaagcgatcctctgaaacgtggttttgcaagctttgttgcaagcaatccgaacgcaccgattcgtaatgcattctatgatgcactgaatagcattgaaccggttaccggtggtggtagcgttcgtaataccctgggttataatgtgaaaaacaaaaacgaattcctgagccagtataaattcaatctgtgctttgaaaacacccagggttatggttatgtgaccgaaaaaatcatcgatgcctatttcagccataccattccgatttattggggtagcccgagcgttgcaaaagatttcaatccgaaaagctttgtgaacgtgcacgacttcaaaaactttgatgaagccatcgattatatcaaatacctgcacacccataaaaacgcctatctggatatgctgtatgaaaatccgctgaatacactggatggtaaagcctatttttaccagaacctgagcttcaaaaaaatcctggcctttttcaaaaccatcctggaaaacgataccatctatcacgataacccgtttatcttttgccgtgatctgaatgaaccgctggttaccattgatgatctgcgtgttaattatgatgacctgcgcgtgaactacgacgatctgcgcatcaattatgatgatctgcgggtaaactatgatgatctgcgtatcaactacgacgacctgcgtgtgaactacgatgacctgcgggttaattatgatgatctgcggattaattatgatgatctgcgtgtgaactatgacgatctgcgtgtgaattacgagcgtctgctgagcaaagcaacccctctgctggaactgagccagaataccaccagtaaaatctatcgtaaagcgtaccagaaaagcctgcctctgctgcgtgcaattcgtcgttgggttaaaaaactgggtctgtaa

SEQ ID NO: 18 - protein, 478 aa, Helicobacter pylori
MFQPLLDAYVESASIEKMASKSPPPLKIAVANWWGDEEIKEFKNSVLYFILSQRYTITLHQNPNEFSDLVFGNPLGSARKILSYQNAKRVFYTGENESPNFNLFDYAIGFDELDFNDRYLRMPLYYDRLHHKAESVNDTTAPYKLKDNSLYALKKPSHCFKEKHPNLCAVVNDESDPLKRGFASFVASNPNAPIRNAFYDALNSIEPVTGGGSVRNTLGYNVKNKNEFLSQYKFNLCFENTQGYGYVTEKIIDAYFSHTIPIYWGSPSVAKDFNPKSFVNVHDFKNFDEAIDYIKYLHTHKNAYLDMLYENPLNTLDGKAYFYQNLSFKKILAFFKTILENDTIYHDNPFIFCRDLNEPLVTIDDLRVNYDDLRVNYDDLRINYDDLRVNYDDLRINYDDLRVNYDDLRVNYDDLRINYDDLRVNYDDLRVNYERLLSKATPLLELSQNTTSKIYRKAYQKSLPLLRAIRRWVKKLGL

SEQ ID NO: 19 - DNA, 978 bp, Azospirillum sp.
atgattgatcagcggaccggcgtgtttctgagcgaatttcttgatacccgcaacagggacccggcagtactggatcgctttctactgcagggaccggatggcggaaggcggggagcgaaaccgaacctgaaagtggcgttttttgacttttggccagaatttgaccccagcgcaaacttttttgtggagattctgagcgcgcgctttcaggtgagcgtggtggaaaacgatagcgatcttgcgattgtgagcgtgtttggcaccggaccacgggaaatacggactgcgcggagcatgttttttaccggagaaaatgtgcggccgccgcttgatggcattgatatgagcgtgagctttgatcgcattgatgacccacggcattttcgcctgccgctatatgtggtgcatgcgtatgatcatctgcgcgagggagcagcaccgtatttttgccagccagtgctgccgccagtgccgccgactcgggaagatgcggcagaacggaagttttgcgcgtttctttataaaaacccaaactgcgcgcgccgcaacgatttttttcatatgcttggcgcgcggcgccatgtggatagcgtgggctggctgctgaacaacaccggcagcgtggtgaaaatgggatggctaccgaaaattcgggtgtttagccgctatcgctttgcgtttgcgtttgaaaacgctagccatccaggctatctgaccgaaaaaattctggatgcttttcaggcgggagcagtgccgctttattggggcgacccaggcgtgctgcgcgacgtggcagcgggcagctttattgatgtgagcaggtatagcagcgatgaagaagcgattgaagcgattctggcgattgatgatgactatggcgcgtatcgccgctatcgcagcactccgccctttcttggcactgaagactttcattttgacgcgtatcgactggcggagtggattgagagccgactataa

SEQ ID NO: 20 - protein, 325 aa, Azospirillum sp.
MIDQRTGVFLSEFLDTRNRDPAVLDRFLLQGPDGGRRGAKPNLKVAFFDFWPEFDPSANFFVEILSARFQVSVVENDSDLAIVSVFGTGPREIRTARSMFFTGENVRPPLDGIDMSVSFDRIDDPRHFRLPLYVVHAYDHLREGAAPYFCQPVLPPVPPTREDAAERKFCAFLYKNPNCARRNDFFHMLGARRHVDSVGWLLNNTGSVVKMGWLPKIRVFSRYRFAFAFENASHPGYLTEKILDAFQAGAVPLYWGDPGVLRDVAAGSFIDVSRYSSDEEAIEAILAIDDDYGAYRRYRSTPPFLGTEDFHFDAYRLAEWIESRL

SEQ ID NO: 21 - DNA, 978 bp, Azospirillum sp.
atgattgacaggcggacaagcgattttctggcggagttcctagcaagcgctaacaaagatccggcagtacttgatcgattcctactacatggaccggaccggggaggccgcagcgcgaaaccgcggctgaaaattgcgttttttgacttttggccggagtttgacccggcagcaaatttttttgtggaaattctgagcgcgcgctttgatctgagcgtggtggataatgatagcgatctagcgattgtgagcgtgtttggaattcgccatcgggaagctcggactgcgcgaagcctgttttttaccggcgaaaatgtgcggcccccgcttgatggcgtggatatgagcgtgagctttgatcgcattgatgacccacggcattatcggctgccgctttatgtgatgcatgcgtgggatcatcggcgcgagggagcaactcggcatttttgccatagcgtgctgccgccggtgcccccgactcgggaagaagcagataggcggaagttttgcgcgtttctttataaaaatccaaactgcgagcgccgcaacgactttttccggatgctttgcgcgcgccgccatgtggaaagcgtgggatggctgctgaacaacaccggcagcgtggtgaaaatgggctggctgccgaaaattcgggtgtttagccgctatcgctttgcgtttgcgtttgaaaatgcgagccatccaggctatctgaccgaaaaaattcttgatgcgtttcaggcgggcgctgtgccgctttattggggggacccaggcgtgctgcgggacgtagcggcgggcagctttattgacgtgagccgctatagcagcgatgaagaagcgattgatgcgattctggcaattgatgacgactatgatacctatcgccgccatcgcagcactgctccatttcttggcactgaagacttttattttgacgcgtttcgactggcggagtggattgagagccgactataa

SEQ ID NO: 22 - protein, 325 aa, Azospirillum sp.
MIDRRTSDFLAEFLASANKDPAVLDRFLLHGPDRGGRSAKPRLKIAFFDFWPEFDPAANFFVEILSARFDLSVVDNDSDLAIVSVFGIRHREARTARSLFFTGENVRPPLDGVDMSVSFDRIDDPRHYRLPLYVMHAWDHRREGATRHFCHSVLPPVPPTREEADRRKFCAFLYKNPNCERRNDFFRMLCARRHVESVGWLLNNTGSVVKMGWLPKIRVFSRYRFAFAFENASHPGYLTEKILDAFQAGAVPLYWGDPGVLRDVAAGSFIDVSRYSSDEEAIDAILAIDDDYDTYRRHRSTAPFLGTEDFYFDAFRLAEWIESRL

SEQ ID NO: 23 - DNA, 978 bp, Azospirillum brasilense
atgcttgaccagcggacaagcgcattcctagaagagtttctggcaaaaccaggcggcgatccggaacggcttgatcgctttctgctgcatggcccatatcgcggacggcgcggcggcaggccacggctgaaactggcgttttatgatttttggccggaatttgatactggcaggaacttttttattgaaattctgagcagccgcttcgacctgagcgtggtggaagatgatagcgaccttgcgattgtgagcgtgtttggcggacggcatcgcgcagcacgcagccggcgcaccctgttttttaccggagaaaatgtgcgccccccgctggatggctttgatatggcagtgagctttgatcgcgtgggcgatccgcgccattatcgcctgccgctttatgtgatgcatgcgtatgaacatatgcgggaaggagcagtgccgcatttttgcagcccagtgctgccgccggtgccgccaagccgggcagcgtttgcagaacgcaacttttgcgcgtttctttataaaaacccgaacggagaacgccgcaaccgcttttttccggcacttgatgcacggcggcgcgtggacagcgtgggctggcatcttaacaacaccggcagcgtggtgaaaatgggctggctggcaaaaattcgcgtgtttgagcgctatcgctttgcgtttgcgtttgaaaacgcgagccatccaggctatctgactgagaaaattcttgatgtgtttcaggcgggagcagtgccgctttattggggggacccagacgtggaacgggaagtggcagcaggcagctttattgatgtgagccgctttgcgactgatgaagaagcagcagaacatattctggcactggatggagactatgatgcgtattgcgcgtatcgcgcggtggcaccatttctgggaactgaagaatttcattttgatgcgtatcgccttgcggattggattgaaagccggctgtag

SEQ ID NO: 24 - protein, 325 aa, Azospirillum brasilense
MLDQRTSAFLEEFLAKPGGDPERLDRFLLHGPYRGRRGGRPRLKLAFYDFWPEFDTGRNFFIEILSSRFDLSVVEDDSDLAIVSVFGGRHRAARSRRTLFFTGENVRPPLDGFDMAVSFDRVGDPRHYRLPLYVMHAYEHMREGAVPHFCSPVLPPVPPSRAAFAERNFCAFLYKNPNGERRNRFFPALDARRRVDSVGWHLNNTGSVVKMGWLAKIRVFERYRFAFAFENASHPGYLTEKILDVFQAGAVPLYWGDPDVEREVAAGSFIDVSRFATDEEAAEHILALDGDYDAYCAYRAVAPFLGTEEFHFDAYRLADWIESRL

SEQ ID NO: 25 - DNA, 912 bp, Azospirillum sp.
atgttagatcggtttctgcttcatgggccggagcgcgggggccgtgcggccagaccgcgcctgaaaattgcgttttttgacttttggccggaatttgacccgagcgcgaatttttttgtagaaattctgagcagccgctttgatgtgagcgtggttgataatgatagcgatttagcgattctgagcgtgtttggcgaacgccaccgcgaagcgcgtaccgcgcgcgcgctgttttttaccggcgaaaatgtgcgcccgccgttagatggcgtggatatgagcgtgagctttgatcgcattgatcatccacgtcattatcgcctgccgttatacgtgatgcatgcgtgggatcaccgtcgcgaaggggcgaccccgcatttttgccatccggtgctgccgccggtgccgccgacgcgtgaagaagcggcgaaacgtaagttttgcgcgtttttatataaaaatcctcactgcgcgcgccgcaacgatttttttcagatgctgtgcgcgcggcgccatgtggaaagcgtgggctggctgctgaataataccggcagcgtggtgaaaatgggctggctgccgaaaattcgcgtgtttgcgcgctatcgctttgcgtttgcgtttgaaaacgcggcgcatccaggctatctgaccgagaaaattctggatgcgtttcaggccgggacggtaccgttatactggggcgacagcggcgtgctgcgcgacgttgcggccggcagctttattgatgtgagccgctatgcgagcgatgaagaagcgattgaagcgattctggcgattgatgatgactatgatagctatcgccggtaccgcggcacggcgccatttttaggcaccgaggacttttactttgacgcgtaccggctggccgagtggattgagagccgcctgtag

SEQ ID NO: 26 - protein, 303 aa, Azospirillum sp.
MLDRFLLHGPERGGRAARPRLKIAFFDFWPEFDPSANFFVEILSSRFDVSVVDNDSDLAILSVFGERHREARTARALFFTGENVRPPLDGVDMSVSFDRIDHPRHYRLPLYVMHAWDHRREGATPHFCHPVLPPVPPTREEAAKRKFCAFLYKNPHCARRNDFFQMLCARRHVESVGWLLNNTGSVVKMGWLPKIRVFARYRFAFAFENAAHPGYLTEKILDAFQAGTVPLYWGDSGVLRDVAAGSFIDVSRYASDEEAIEAILAIDDDYDSYRRYRGTAPFLGTEDFYFDAYRLAEWIESRL

SEQ ID NO: 27 - DNA, 1032 bp, Butyrivibrio sp.
atgaatataattcacttttatgcaagatatttaagagaatcacataactggaacagagaacgtgaagttactcgtaacggtgttatgacttttgctaattggtggagagaagacccgcacaagaactggtttgcaagatttattgacgctggaagcaaagaccctgaacgcaggattaggttctatagtatttttggaccatatagtaaattaaaagaagattttgatggagctaagatattcttttccggagagaatcttgagcagccggtctatcacagaatattaaagacagatcctatagaagatagaatatgggctgacagaaggaagctgtatggtaattatggagcaggagatgtggatcttgctataggatttggcaatagggaagaggattcacttatgggatttgaaggaagcaggaagactaaatatatccgctttcctttatggcttacatatgtttttgatcctgattgtactcatgatgatattaagagaaccattgatgagataaatgcagttcgttccacaggcaggaaggatactctgcttcttgcatcgcatgatttctgggggacaaggtcagatatcttaaagagtctagaaggtgtatgcgatgttagtattgccggtaaatggcgcaacaacaccaaagaactctgggaagattataacaatgacaagaataaatatctgtcagaatttaaatttaatatatgccctgaaaatgttgatgcaccgggatatgtgacagagaagatatttgatgcttttaaatgcggagctattcctatatatcagggctgtcttggtaagcctgagccggatgtgataaatacagatgcagttcttttatgggacttcgatggagataattcagatactatatccttgattaaaaaactaaattcggataatgtatactatgataactttgtatctcagcccaaattcaaaccggatgcggcagagtatgtggttgcatgtatggatgagctgaggcgaagctttgatcagctcatctga

SEQ ID NO: 28 - protein, 343 aa, Butyrivibrio sp.
MNIIHFYARYLRESHNWNREREVTRNGVMTFANWWREDPHKNWFARFIDAGSKDPERRIRFYSIFGPYSKLKEDFDGAKIFFSGENLEQPVYHRILKTDPIEDRIWADRRKLYGNYGAGDVDLAIGFGNREEDSLMGFEGSRKTKYIRFPLWLTYVFDPDCTHDDIKRTIDEINAVRSTGRKDTLLLASHDFWGTRSDILKSLEGVCDVSIAGKWRNNTKELWEDYNNDKNKYLSEFKFNICPENVDAPGYVTEKIFDAFKCGAIPIYQGCLGKPEPDVINTDAVLLWDFDGDNSDTISLIKKLNSDNVYYDNFVSQPKFKPDAAEYVVACMDELRRSFDQLI

SEQ ID NO: 29 - DNA, 1059 bp, Porphyromonas catoniae
atgctagccccatacaaaagccctatctttgtgcccatatacgacactaaggcaatgaatccccccaccaaacaaccacttagagagaggctccacatgatgcgtaggcgcaatcgtatccgaaaacgctctgtgatagctctcatcaaatctcaccttgacagctcacgctaccaggactacaactggtgggacagtcacgcctcgaccttttggctgccacggttcattgacctacacctcgagcccaagaagaggatcaatctcttctcttgcttccaaaatcccctaatgctcatccgctactacaaaggggtgaagatcttcctatcaggtgagaaccttgccaataacgagcactttggcttccatccccgcatgctcgatcataggatcaacgaggtggacttagccctaggctttgagttccgcaaggatcccaagtactatcgcttccccctttggatctatcagaatgagttcatcagccccagtgctagcctagaggatatacgtgcgctccttgagcagatcaacgatccctccacccgtcgtagcacgggacgcagtcgcttcatcgggcagatctccagccacgacaaaggcggaatgcgaggacggctcattgacctcctgaatcccatcggacaaatcgactgcgcagggaagttccgtcacaacaccgatgagctcctcgaggtctacggggatgacaagtttaagtacctagctaactaccgcttcaacctctgcccagagaattcactaggcgaaggctacatcaccgagaaggtcttcgacagcatacgcgcaggctgtatccccatctattggggtgcttacctagagcctggcatccttaaccccaaggctatcctacgcttcgaggaagggaaggaacaagagttctataaccgagtgaaggagctgtgggagaacgaagcggcctacgagcagtttatcctcgagccccccttcgtagagggagcagcagagcgcatctgggaaatcctccaggggcttcgtgagcgccttgcccctcttgtggaggaaggataa

SEQ ID NO: 30 - protein, 352 aa, Porphyromonas catoniae
MLAPYKSPIFVPIYDTKAMNPPTKQPLRERLHMMRRRNRIRKRSVIALIKSHLDSSRYQDYNWWDSHASTFWLPRFIDLHLEPKKRINLFSCFQNPLMLIRYYKGVKIFLSGENLANNEHFGFHPRMLDHRINEVDLALGFEFRKDPKYYRFPLWIYQNEFISPSASLEDIRALLEQINDPSTRRSTGRSRFIGQISSHDKGGMRGRLIDLLNPIGQIDCAGKFRHNTDELLEVYGDDKFKYLANYRFNLCPENSLGEGYITEKVFDSIRAGCIPIYWGAYLEPGILNPKAILRFEEGKEQEFYNRVKELWENEAAYEQFILEPPFVEGAAERIWEILQGLRERLAPLVEEG

SEQ ID NO: 31 - DNA, 1032 bp, Butyrivibrio fibrisolvens
atgaacataattcacttttatgcaagatatttaagagaatcacataattggaacagagaacgtgaagttactcgtaatggtgttatgacttttgctaactggtggagagaagatccgcataagaactggtttgcaagatttattgatgctgggaataaagaccctgagcgcaggatcagattctatagtatttttggaccttatagtaaattgaaggaagattttgatggagccaagatattcttttccggagaaaatcttgaacagccggttttacacagaatactaaagacagatcctatagaagacaggatatgggctgacagaagaaagctgtatggtaattatggagctggagaagtggatcttgctataggttttggtaatagagaagaggattcacttctgggatttgaagggagcaggaagacaaaatatatccgctttcctttatggcttacatatgtctttgatcctgactgtactcatgatgatattaagagaaccatagatgagataaatgcagttcgttctacaggcaggaaggataccctccttcttgcatcgcatgatttctgggggacaaggtcagatatcttaaagagtcttgaaggtgtatgtgatattagtattgccggtaaatggcgcaataacaccaaagagctctgggaagattatgacaatgacaagaataaatatctgtcagaatttaaatttaacatatgccctgaaaatgttgatgcaccgggatatgtaacagagaagatatttgatgcttttaaatgcggagctattcctatatatcagggctgcctaggtaagcctgagccgaatgtgataaatacagatgcagtacttctatgggacttcgatggagataattcagatactatagccttgattaaaaaactaaattcggataatgtatactatgataactttgtatctcagcctaaattcaaaccggatgcggcagagtatgtagttgcatgtatggatgagctaaggcgtagctttgacaggctgatctga

SEQ ID NO: 32 - protein, 343 aa, Butyrivibrio fibrisolvens
MNIIHFYARYLRESHNWNREREVTRNGVMTFANWWREDPHKNWFARFIDAGNKDPERRIRFYSIFGPYSKLKEDFDGAKIFFSGENLEQPVLHRILKTDPIEDRIWADRRKLYGNYGAGEVDLAIGFGNREEDSLLGFEGSRKTKYIRFPLWLTYVFDPDCTHDDIKRTIDEINAVRSTGRKDTLLLASHDFWGTRSDILKSLEGVCDISIAGKWRNNTKELWEDYDNDKNKYLSEFKFNICPENVDAPGYVTEKIFDAFKCGAIPIYQGCLGKPEPNVINTDAVLLWDFDGDNSDTIALIKKLNSDNVYYDNFVSQPKFKPDAAEYVVACMDELRRSFDRLI

SEQ ID NO: 33 - protein, 5 aa, Artificial sequence
  conserved GDP-fucose binding domain
  VARIANT (1)..(1): Xaa can be Tyr, Trp, Leu, His, Phe or Met
  UNSURE (2)..(2): Xaa can be any naturally occuring amino acid
  VARIANT (3)..(3): Xaa can be Thr, Ser or Cys
  VARIANT (4)..(4): Xaa can be Glu, Gln, Asp or Asn
  VARIANT (5)..(5): Xaa can be Lys or Arg
XXXXX

SEQ ID NO: 34 - protein, 6 aa, Artificial sequence
  conserved domain
  VARIANT (1)..(1): Xaa can be Lys or Asp
  VARIANT (2)..(2): Xaa can be Leu, Lys or Met
  UNSURE (3)..(5): Xaa can be any naturally occurring amino acid
  VARIANT (6)..(6): Xaa can be Phe or Tyr
XXXXXX

SEQ ID NO: 35 - protein, 8 aa, Artificial sequence
  conserved motif
  VARIANT (1)..(1): Xaa can be Asn or His
  UNSURE (2)..(2): Xaa can be any naturally occurring amino acid
  UNSURE (6)..(6): Xaa can be any naturally occurring amino acid
XXDPAXLD

SEQ ID NO: 36 - protein, 6 aa, Artificial sequence
  conserved motif
  VARIANT (3)..(3): Xaa can be Ala or Ser
DMXVSF

SEQ ID NO: 37 - protein, 5 aa, Artificial sequence
  conserved GDP-fucose binding domain
  UNSURE (2)..(2): Xaa can be any naturally occurring amino acid
YXTEK

SEQ ID NO: 38 - protein, 6 aa, Artificial sequence
  conserved domain
  VARIANT (1)..(1): Xaa can be Lys or Asp
  UNSURE (3)..(3): Xaa can be any naturally occurring amino acid
  VARIANT (4)..(4): Xaa can be Ile, Leu or Met
  VARIANT (6)..(6): Xaa can be Phe or Tyr
XLXXGX

SEQ ID NO: 39 - protein, 6 aa, Artificial sequence
  conserved domain
  VARIANT (1)..(1): Xaa can be Lys or Asp
  VARIANT (2)..(2): Xaa can be Leu or Lys
  UNSURE (3)..(3): Xaa can be any naturally occurring amino acid
  VARIANT (5)..(5): Xaa can be Ser or Gly
  VARIANT (6)..(6): Xaa can be Phe or Tyr
XXXLXX

SEQ ID NO: 40 - protein, 6 aa, Artificial sequence
  conserved domain
  VARIANT (1)..(1): Xaa can be Lys or Asp
  UNSURE (3)..(3): Xaa can be any naturally occurring amino acid
  UNSURE (6)..(6): Xaa can be Phe or Tyr
XLXLGX

SEQ ID NO: 41 - protein, 8 aa, Artificial sequence
  conserved domain
  VARIANT (2)..(2): Xaa can be Ile or Val
  VARIANT (4)..(4): Xaa can be Phe or Leu
  USNURE (5)..(5): Xaa can be any naturally occurring amino acid
KXFXXGEN

SEQ ID NO: 42 - protein, 5 aa, Artificial sequence
  conserved domain
RFPLW

(19) United States
(12) Patent Application Publication
(10) Pub. No.: US 2022/0002773 A1
(43) Pub. Date: Jan. 6, 2022

(54) PRODUCTION OF 3-FUCOSYLLACTOSE AND LACTOSE CONVERTING ALPHA-1,3-FUCOSYLTRANSFERASE ENZYMES

(71) Applicant: INBIOSE N.V., Gent (BE)

(72) Inventors: Joeri Beauprez, Bredene (BE); Nausicaä Lannoo, Eke-Nazareth (BE); Kristof Vandewalle, Tielt (BE); Annelies Vercauteren, Eke (BE)

(21) Appl. No.: 17/295,688

(22) PCT Filed: Dec. 18, 2019
(86) PCT No.: PCT/EP2019/085841

§ 371 Date: May 20, 2021

(30) Foreign Application Priority Data
Dec. 18, 2018 (EP) 18213728.1

(51) Int. Cl. C12P 19/18; C12N 9/10; C12N 15/70; C12N 15/81; C12N 1/18; C12N 1/20

(52) U.S. Cl. CPC C12P 19/18; C12N 9/1051; C12N 15/70; C12Y 204/01065; C12N 1/18; C12N 1/20; C12N 15/81

(57) ABSTRACT

Methods for producing 3-fucosyllactose (3-FL) as well as novel fucosyltransferases, more specifically novel lactose binding alpha-1,3-fucosyltransferase polypeptides, and their applications. Furthermore, methods are provided for producing 3-fucosyllactose (3-FL) using the novel lactose binding alpha-1,3-fucosyltransferases.

### CROSS-REFERENCE TO RELATED APPLICATIONS

This application is a national phase entry under 35 U.S.C. § 371 of International Patent Application PCT/EP2019/085841, filed Dec. 18, 2019, designating the United States of America and published in English as International Patent Publication WO 2020/127417 A2 on Jun. 25, 2020, which claims the benefit under Article 8 of the Patent Cooperation Treaty to European Patent Application Serial No. 18213728.1, filed Dec. 18, 2019.

### TECHNICAL FIELD

The present disclosure relates to methods for producing 3-fucosyllactose (3-FL) as well as newly identified fucosyltransferases, more specifically newly identified lactose binding alphα-1,3-fucosyltransferase polypeptides, and their applications. Furthermore, the present disclosure provides methods for producing 3-fucosyllactose (3-FL) using the newly identified lactose binding alphα-1,3-fucosyltransferases.

### BACKGROUND

Today, more than 80 compounds belonging to the family of Human Milk Oligosaccharides (HMOs), have been structurally characterized. These HMOs represent a class of complex oligosaccharides that function as prebiotics. Additionally, the structural homology of HMO to epithelial epitopes accounts for protective properties against bacterial pathogens. Within the infant gastrointestinal tract, HMOs selectively nourish the growth of selected bacterial strains and are, thus, priming the development of a unique gut microbiota in breast milk-fed infants.

Some of these Human Milk oligosaccharides require the presence of particular fucosylated structures that most likely exhibit a particular biological activity. Production of these fucosylated oligosaccharides requires the action of a fucosyltransferase. Such fucosyltransferases, which belong to the enzyme family of glycosyltransferases, are widely expressed in vertebrates, invertebrates, plants, fungi, yeasts and bacteria. They catalyze the transfer of a fucose residue from a donor, generally guanosine-diphosphate fucose (GDP-fucose) to an acceptor, which include oligosaccharides, (glyco)proteins and (glyco)lipids. The thus fucosylated acceptor substrates are involved in a variety of biological and pathological processes.

Several fucosyltransferases have been identified, e.g., in the bacteria Helicobacter pylori, Escherichia coli, Salmonella enterica, in mammals, Caenorhabditis elegans and Schistosoma mansoni, as well as in plants.

Fucosyltransferases are classified based on the site of fucose addition into, for example, alphα-1,2, alphα-1,3, alphα-1,4 and O-fucosyltransferases.

Several alphα-1,3-fucosyltransferases are already described in the art. WO 1998/055630 describes a bacterial alphα-1,3-fucosyltransferase gene of Helicobacter pylori that can be used in the production of oligosaccharides such as Lewis X, Lewis Y, and sialyl Lewis X. WO 2016/040531 describes several alphα-1,3-fucosyltransferases for the production of fucosylated oligosaccharides. Here, α-1,3-fucosyltransferases are described with 25 to 100% sequence identity to the Bacteroides nordii CafC enzyme. However, in Table 1 of that filing, the authors clearly show that over half (7 out of 12) of their tested enzymes, many of which with >25% sequence identity to CafC, are unable to produce 3-fucosyllactose using lactose as the acceptor substrate. This illustrates that clearly not all hypothetical fucosyltransferases indeed have lactose binding fucosyltransferase activity. WO2012/049083 describes some new alphα-1,3-fucosyltransferases and their use for the production of fucosylated products. Huang et al. 2017 did a comparison of various exogenous alphα-1,3-fucosyltransferase candidates, as well as a series of E. coli host strains, and demonstrated that futA from Helicobacter pylori using E. coli BL21(DE3) as the host strain yielded the highest titers of 3-fucosyllactose, one of the Human Milk Oligosaccharides.

In general, alphα-1,3-fucosyltransferases, also known as 3-fucosyltransferases or 3-fucosyltransferase enzymes are known to have low affinity for lactose. A 3-fucosyltransferase is needed for the production of the HMO 3-fucosyllactose. The low affinity has a negative effect on the productivity of 3-fucosyllactose. In order to improve conversion rates and productivity, there is need for transferases with sufficient lactose affinity, preferably higher lactose affinity.

Thus, provided herein are tools and methods by means of which 3-fucosyllactose can be produced or synthesized in an efficient, time and cost-effective way and that yields similar or higher amounts of the desired product compared to state of the art methods.

### STATEMENT ACCORDING TO 37 C.F.R. § 1.821(c) or (e)-SEQUENCE LISTING SUBMITTED AS A TXT AND PDF FILES

Pursuant to 37 C.F.R. § 1.821(c) or (e), files containing a TXT version and a PDF version of the Sequence Listing have been submitted concomitant with this application, the contents of which are hereby incorporated by reference.

### BRIEF SUMMARY

Surprisingly, it has now been found that the newly identified lactose binding alphα-1,3-fucosyltransferase enzymes of the disclosure provide for transferases with similar or higher lactose binding and/or transferase properties than the presently known lactose binding alphα-1,3 -fucosyltransferase enzymes.

The disclosure, therefore, provides methods for producing 3-fucosyllactose (3FL) using the newly identified lactose binding alphα-1,3-fucosyltransferases. The 3FL can be obtained by reacting lactose in the presence of alphα-1,3-fucosyltransferase, capable of catalyzing the formation of the 3-fucosyllactose oligosaccharides from lactose and GDP-fucose. Alternatively, it can also be obtained from a microorganism producing an alphα-1,3-fucosyltransferase according to the present disclosure.

### Definitions

The words used in this specification to describe the various embodiments of this disclosure are to be understood, not only in the sense of their commonly defined meanings, but to include by special definition in this specification, structure, material or acts beyond the scope of the commonly defined meanings. Thus, if an element can be understood in the context of this specification as including more than one meaning, then its use in a claim must be understood as being generic to all possible meanings supported by the specification and by the word itself

The various embodiments and aspects of embodiments disclosed herein are to be understood not only in the order and context specifically described in this specification, but to include any order and any combination thereof. Whenever the context requires, all words used in the singular number shall be deemed to include the plural and vice versa. Unless defined otherwise, all technical and scientific terms used herein generally have the same meaning as commonly understood by one of ordinary skill in the art to which this disclosure belongs. Generally, the nomenclature used herein and the laboratory procedures in cell culture, molecular genetics, organic chemistry and nucleic acid chemistry and hybridization described herein are those well-known and commonly employed in the art. Standard techniques are used for nucleic acid and peptide synthesis. Generally, enzymatic reactions and purification steps are performed according to the manufacturer's specifications.

In the drawings and specification, there have been disclosed embodiments of the invention, and although specific terms are employed, the terms are used in a descriptive sense only and not for purposes of limitation, the scope of the invention being set forth in the following claims. It must be understood that the illustrated embodiments have been set forth only for the purposes of example and that it should not be taken as limiting the invention. It will be apparent to those skilled in the art that alterations, other embodiments, improvements, details and uses can be made consistent with the letter and spirit of the disclosure herein and within the scope of this disclosure, which is limited only by the claims, construed in accordance with the patent law, including the doctrine of equivalents. In the claims that follow, reference characters used to designate claim steps are provided for convenience of description only, and are not intended to imply any particular order for performing the steps.

According to the disclosure, the term “polynucleotide(s)” generally refers to any polyribonucleotide or polydeoxyribonucleotide, which may be unmodified RNA or DNA or modified RNA or DNA. “Polynucleotide(s)” include, without limitation, single- and double-stranded DNA, DNA that is a mixture of single- and double-stranded regions or single-, double- and triple-stranded regions, single- and double-stranded RNA, and RNA that is mixture of single- and double-stranded regions, hybrid molecules comprising DNA and RNA that may be single-stranded or, more typically, double-stranded, or triple-stranded regions, or a mixture of single- and double-stranded regions. In addition, “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. As used herein, the term “polynucleotide(s)” also includes DNAs or RNAs as described above that contain one or more modified bases. Thus, DNAs or RNAs with backbones modified for stability or for other reasons are “polynucleotide(s)” according to the disclosure. Moreover, DNAs or RNAs comprising unusual bases, such as inosine, or modified bases, such as tritylated bases, are to be understood to be covered by the term “polynucleotides.” It will be appreciated that a great variety of modifications have been made to DNA and RNA that serve many useful purposes known to those of skill in the art. The term “polynucleotide(s)” as it is employed herein embraces such chemically, enzymatically or metabolically modified forms of polynucleotides, as well as the chemical forms of DNA and RNA characteristic of viruses and cells, including, for example, simple and complex cells. The term “polynucleotide(s)” also embraces short polynucleotides often referred to as oligonucleotide(s).

“Polypeptide(s)” refers to any peptide or protein comprising two or more amino acids joined to each other by peptide bonds or modified peptide bonds. “Polypeptide(s)” refers to both short chains, commonly referred to as peptides, oligopeptides and oligomers and to longer chains generally referred to as proteins. Polypeptides may contain amino acids other than the 20 gene encoded amino acids. “Polypeptide(s)” include those modified either by natural processes, such as processing and other post-translational modifications, but also by chemical modification techniques. Such modifications are well described in basic texts and in more detailed monographs, as well as in a voluminous research literature, and they are well known to the skilled person. The same type of modification may be present in the same or varying degree at several sites in a given polypeptide. Furthermore, a given polypeptide may contain many types of modifications. Modifications can occur anywhere in a polypeptide, including the peptide backbone, the amino acid side-chains, and the amino or carboxyl termini. Modifications include, for example, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent cross-links, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, lipid attachment, sulfation, gamma-carboxylation of glutamic acid residues, hydroxylation and ADP-ribosylation, selenoylation, transfer-RNA mediated addition of amino acids to proteins, such as arginylation, and ubiquitination. Polypeptides may be branched or cyclic, with or without branching. Cyclic, branched and branched circular polypeptides may result from post-translational natural processes and may be made by entirely synthetic methods, as well.

“Isolated” means altered “by the hand of man” from its natural state, i.e., if it occurs in nature, it has been changed or removed from its original environment, or both. For example, a polynucleotide or a polypeptide naturally present in a living organism is not “isolated,” but the same polynucleotide or polypeptide separated from the coexisting materials of its natural state is “isolated,” as the term is employed herein. Similarly, a “synthetic” sequence, as the term is used herein, means any sequence that has been generated synthetically and not directly isolated from a natural source. “Synthesized,” as the term is used herein, means any synthetically generated sequence and not directly isolated from a natural source.

“Recombinant” means genetically engineered DNA prepared by transplanting or splicing genes from one species into the cells of a host organism of a different species. Such DNA becomes part of the host's genetic makeup and is replicated. “Mutant” cell or microorganism as used within the context of the present disclosure refers to a cell or microorganism that is genetically engineered or has an altered genetic make-up.

The terms “cell genetically modified for the production of 3-fucosyllactose” within the context of the present disclosure refers to a cell of a microorganism that is genetically manipulated to comprise at least one of i) a recombinant gene encoding an a 1,3 fucosyltransferase necessary for the synthesis of 3-fucosyllactose, ii) a biosynthetic pathway to produce a GDP-fucose suitable to be transferred by fucosyltransferase to lactose, and/or iii) a biosynthetic pathway to produce lactose or a mechanism of internalization of lactose from the culture medium into the cell where it is fucosylated to produce the 3-fucosyllactose.

The terms “nucleic acid sequence coding for an enzyme for 3-fucosyllactose synthesis” relates to nucleic acid sequences coding for enzymes necessary in the synthesis pathway to 3-fucosyllactose, e.g., an enzyme able to transfer the fucose moiety of a GDP-fucose donor substrate onto the 3 hydroxyl group of the galactose moiety of lactose and thus producing 3-fucosyllactose.

The term “endogenous,” within the context of the present disclosure refers to any polynucleotide, polypeptide or protein sequence that is a natural part of a cell and is occurring at its natural location in the cell chromosome. The term “exogenous” refers to any polynucleotide, polypeptide or protein sequence that originates from outside the cell under study and not a natural part of the cell or that is not occurring at its natural location in the cell chromosome or plasmid.

The term “heterologous” when used in reference to a polynucleotide, gene, nucleic acid, polypeptide, or enzyme refers to a polynucleotide, gene, nucleic acid, polypeptide, or enzyme that is from a source or derived from a source other than the host organism species. In contrast a “homologous” polynucleotide, gene, nucleic acid, polypeptide, or enzyme is used herein to denote a polynucleotide, gene, nucleic acid, polypeptide, or enzyme that is derived from the host organism species. When referring to a gene regulatory sequence or to an auxiliary nucleic acid sequence used for maintaining or manipulating a gene sequence (e.g., a promoter, a 5′ untranslated region, 3′ untranslated region, poly A addition sequence, intron sequence, splice site, ribosome binding site, internal ribosome entry sequence, genome homology region, recombination site, etc.), “heterologous” means that the regulatory sequence or auxiliary sequence is not naturally associated with the gene with which the regulatory or auxiliary nucleic acid sequence is juxtaposed in a construct, genome, chromosome, or episome. Thus, a promoter operably linked to a gene to which it is not operably linked to in its natural state (i.e., in the genome of a non-genetically engineered organism) is referred to herein as a “heterologous promoter,” even though the promoter may be derived from the same species (or, in some cases, the same organism) as the gene to which it is linked.

The term “polynucleotide encoding a polypeptide” as used herein encompasses polynucleotides that include a sequence encoding a polypeptide of the disclosure, particularly an α-1,3-fucosyltransferase having the amino acid sequence as set forth in SEQ ID NOS: 2, 4, 6, 8, 10, 12, 14, 16, 20, 22, 28, 30 or 32 of the attached sequence listing. For sake of clarity, also the polynucleotide encoding the polypeptides of SEQ ID NOS: 18, 24 and 26 is a polynucleotide encompassed by the definition, but the polynucleotide of SEQ ID NO: 18 is a prior art α-1,3-fucosyltransferase used as a reference and the polynucleotides of SEQ ID NOS: 24 and 26 are α-1,3-fucosyltransferase enzymes that are non-functional towards lactose as acceptor. The term also encompasses polynucleotides that include a single continuous region or discontinuous regions encoding the polypeptide (for example, interrupted by integrated phage or an insertion sequence or editing) together with additional regions that also may contain coding and/or non-coding sequences.

“Variant(s)” as the term is used herein, is a polynucleotide or polypeptide that differs from a reference polynucleotide or polypeptide respectively but retains essential properties. A typical variant of a polynucleotide differs in nucleotide sequence from another, reference polynucleotide. Changes in the nucleotide sequence of the variant may or may not alter the amino acid sequence of a polypeptide encoded by the reference polynucleotide. Nucleotide changes may result in amino acid substitutions, additions, deletions, fusions and truncations in the polypeptide encoded by the reference sequence, as discussed below. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more substitutions, additions, deletions in any combination. A substituted or inserted amino acid residue may or may not be one encoded by the genetic code. A variant of a polynucleotide or polypeptide may be a naturally occurring such as an allelic variant, or it may be a variant that is not known to occur naturally. Non-naturally occurring variants of polynucleotides and polypeptides may be made by mutagenesis techniques, by direct synthesis, and by other recombinant methods known to the persons skilled in the art. In some embodiments, the present disclosure contemplates making functional variants by modifying the structure of a membrane protein as used in the present disclosure. Variants can be produced by amino acid substitution, deletion, addition, or combinations thereof. For instance, it is reasonable to expect that an isolated replacement of a leucine with an isoleucine or valine, an aspartate with a glutamate, a threonine with a serine, or a similar replacement of an amino acid with a structurally related amino acid (e.g., conservative mutations) will not have a major effect on the biological activity of the resulting molecule. Conservative replacements are those that take place within a family of amino acids that are related in their side chains. Whether a change in the amino acid sequence of a polypeptide of the disclosure results in a functional homolog can be readily determined by assessing the ability of the variant polypeptide to produce a response in cells in a fashion similar to the wild-type polypeptide, as in the case of the present disclosure, to provide better yield, productivity, and/or growth speed than a cell without the variant.

The term “functional homolog” as used herein describes those molecules that have sequence similarity and also share at least one functional characteristic such as a biochemical activity. Functional homologs will typically give rise to the same characteristics to a similar, but not necessarily the same, degree. Functionally homologous proteins give the same characteristics where the quantitative measurement produced by one homolog is at least 10 percent of the other; more typically, at least 20 percent, between about 30 percent and about 40 percent; for example, between about 50 percent and about 60 percent; between about 70 percent and about 80 percent; or between about 90 percent and about 95 percent; between about 98 percent and about 100 percent, or greater than 100 percent of that produced by the original molecule. Thus, where the molecule has enzymatic activity the functional homolog will have the above-recited percent enzymatic activities compared to the original enzyme. Where the molecule is a DNA-binding molecule (e.g., a polypeptide) the homolog will have the above-recited percentage of binding affinity as measured by weight of bound molecule compared to the original molecule.

A functional homolog and the reference polypeptide may be naturally occurring polypeptides, and the sequence similarity may be due to convergent or divergent evolutionary events. Functional homologs are sometimes referred to as orthologs, where “ortholog” refers to a homologous gene or protein that is the functional equivalent of the referenced gene or protein in another species.

Functional homologs can be identified by analysis of nucleotide and polypeptide sequence alignments. For example, performing a query on a database of nucleotide or polypeptide sequences can identify homologs of biomass-modulating polypeptides. Sequence analysis can involve BLAST, Reciprocal BLAST, or PSI-BLAST analysis of non-redundant databases using amino acid sequence of a biomass-modulating polypeptide as the reference sequence. Amino acid sequence is, in some instances, deduced from the nucleotide sequence. Typically, those polypeptides in the database that have greater than 40 percent sequence identity are candidates for further evaluation for suitability as a biomass-modulating polypeptide. Amino acid sequence similarity allows for conservative amino acid substitutions, such as substitution of one hydrophobic residue for another or substitution of one polar residue for another. If desired, manual inspection of such candidates can be carried out in order to narrow the number of candidates to be further evaluated. Manual inspection can be performed by selecting those candidates that appear to have domains present in productivity-modulating polypeptides, e.g., conserved functional domains.

“Fragment,” with respect to a polynucleotide, refers to a clone or any part of a polynucleotide molecule, particularly a part of a polynucleotide that retains a usable, functional characteristic. Useful fragments include oligonucleotides and polynucleotides that may be used in hybridization or amplification technologies or in the regulation of replication, transcription or translation. A “polynucleotide fragment” refers to any subsequence of a polynucleotide, typically, of at least about nine consecutive nucleotides, for example, at least about 30 nucleotides or at least about 50 nucleotides of any of the sequences provided herein. Exemplary fragments can additionally or alternatively include fragments that comprise, consist essentially of, or consist of a region that encodes a conserved family domain of a polypeptide. Exemplary fragments can additionally or alternatively include fragments that comprise a conserved domain of a polypeptide.

Fragments may additionally or alternatively include subsequences of polypeptides and protein molecules, or a subsequence of the polypeptide. In some cases, the fragment or domain is a subsequence of the polypeptide that performs at least one biological function of the intact polypeptide in substantially the same manner, or to a similar extent, as does the intact polypeptide. For example, a polypeptide fragment can comprise a recognizable structural motif or functional domain such as a DNA-binding site or domain that binds to a DNA promoter region, an activation domain, or a domain for protein-protein interactions, and may initiate transcription. Fragments can vary in size from as few as three amino acid residues to the full length of the intact polypeptide, for example, at least about 20 amino acid residues in length, for example, at least about 30 amino acid residues in length. Preferentially a fragment is a functional fragment that has at least one property or activity of the polypeptide from which it is derived, such as, for example, the fragment can include a functional domain or conserved domain of a polypeptide. A domain can be characterized, for example, by a Pfam or Conserved Domain Database (CDD) designation.

The terms “alphα-1,3-fucosyltransferase,” “alpha 1,3 fucosyltransferase,” “3-fucosyltransferase,” “α-1,3-fucosyltransferase,” “α 1,3 fucosyltransferase,” “3 fucosyltransferase,” “3-FT” or “3FT” as used in the present disclosure, are used interchangeably and refer to a glycosyltransferase that catalyzes the transfer of fucose from the donor substrate GDP-L-fucose, to the acceptor molecule lactose in an alphα-1,3-linkage. A polynucleotide encoding an “alphα-1,3-fucosyltransferase” or any of the above terms, refers to a polynucleotide encoding such glycosyltransferase that catalyzes the transfer of fucose from the donor substrate GDP-L-fucose, to the acceptor molecule lactose in an alphα-1,3-linkage.

The terms “3-fucosyllactose,” “alphα-1,3-fucosyllactose,” “alpha 1,3 fucosyllactose,” “α-1,3-fucosyllactose,” “α 1,3 fucosyllactose,” “Galβ-4(Fucα1-3)G1c,” “3FL” or “3-FL” as used in the present disclosure, are used interchangeably and refer to the product obtained by the catalysis of the alphα-1,3-fucosyltransferase transferring the fucose residue from GDP-L-fucose to lactose in an alphα-1,3-linkage.

“Oligosaccharide” as the term is used herein and as generally understood in the state of the art, refers to a saccharide polymer containing a small number, typically three to ten, of simple sugars, i.e., monosaccharides.

The term “purified” refers to material that is substantially or essentially free from components that interfere with the activity of the biological molecule. For cells, saccharides, nucleic acids, and polypeptides, the term “purified” refers to material that is substantially or essentially free from components that normally accompany the material as found in its native state. Typically, purified saccharides, oligosaccharides, proteins or nucleic acids of the disclosure are at least about 50%, 55%, 60%, 65%, 70%, 75%, 80% or 85% pure, usually at least about 90%, 91%, 92%, 93%, 94%, 95%, 96%, 97%, 98%, or 99% pure as measured by band intensity on a silver stained gel or other method for determining purity. Purity or homogeneity can be indicated by a number of means well known in the art, such as polyacrylamide gel electrophoresis of a protein or nucleic acid sample, followed by visualization upon staining. For certain purposes high resolution will be needed and HPLC or a similar means for purification utilized. For oligosaccharides, e.g., 3-fucosyllactose, purity can be determined using methods such as but not limited to thin layer chromatography, gas chromatography, NMR, HPLC, capillary electrophoresis or mass spectroscopy.

The terms “identical” or percent “identity” or % “identity” in the context of two or more nucleic acid or polypeptide sequences, refer to two or more sequences or subsequences that are the same or have a specified percentage of amino acid residues or nucleotides that are the same, when compared and aligned for maximum correspondence, as measured using sequence comparison algorithms or by visual inspection. For sequence comparison, one sequence acts as a reference sequence, to which test sequences are compared. When using a sequence comparison algorithm, test and reference sequences are inputted into a computer, subsequence coordinates are designated, if necessary, and sequence algorithm program parameters are designated. The sequence comparison algorithm then calculates the percent sequence identity for the test sequence(s) relative to the reference sequence, based on the designated program parameters. Percent identity can be determined using BLAST and PSI-BLAST (Altschul et al., 1990, J. Mol. Biol. 215:3, 403-410; Altschul et al., 1997, Nucleic Acids Res. 25:17, 3389-402). For the purposes of this disclosure, percent identity is determined using MatGAT2.01 (Campanella et al., 2003, BMC Bioinformatics 4:29). MatGAT utilizes a Myers and Miller global alignment algorithm for conducting pairwise alignments. The following default parameters for protein are employed: (1) Gap cost Existence: 12 and Extension: 2; (2) The Matrix employed was BLOSUM50.

The term “control sequences” refers to sequences recognized by the host cells transcriptional and translational systems, allowing transcription and translation of a polynucleotide sequence to a polypeptide. Such DNA sequences are thus necessary for the expression of an operably linked coding sequence in a particular host cell or organism. Such control sequences can be, but are not limited to, promoter sequences, ribosome binding sequences, Shine Dalgarno sequences, Kozak sequences, transcription terminator sequences. The control sequences that are suitable for prokaryotes, for example, include a promoter, optionally an operator sequence, and a ribosome binding site. Eukaryotic cells are known to utilize promoters, polyadenylation signals, and enhancers. DNA for a presequence or secretory leader may be operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it affects the transcription of the sequence; or a ribosome binding site is operably linked to a coding sequence if it is positioned so as to facilitate translation. Control sequences can, furthermore, be controlled with external chemicals, such as, but not limited to, IPTG, arabinose, lactose, allo-lactose, rhamnose or fucose via an inducible promoter or via a genetic circuit that either induces or represses the transcription or translation of the polynucleotide to a polypeptide.

The term “end of fermentation” as used in the present disclosure refers to the time at which a fermentation is harvested for product purification.

Generally, “operably linked” means that the DNA sequences being linked are contiguous, and, in the case of a secretory leader, contiguous and in reading phase. However, enhancers do not have to be contiguous.

According to a first embodiment, the disclosure provides a method for producing α-1,3-fucosyllactose. The method comprising the steps of:
a) providing a polypeptide with α-1,3-fucosyltransferase activity and with the ability to use lactose as acceptor substrate wherein the polypeptide comprises:
   i) an amino acid sequence encoding a conserved GDP-fucose binding domain [Y/W/L/H/F/M]X[T/S/C][E/Q/D/A][K/R] (SEQ ID NO: 33);
   ii) an amino acid sequence encoding a conserved [K/D][L/K/M]XXX[F/Y] domain (SEQ ID NO: 34), and
   iii) wherein additionally the conserved motif [N/H]XDPAXLD (SEQ ID NO: 35) is present at the N-terminal region if the domain of ii) equals DM[A/S]VSF (SEQ ID NO: 36);
wherein X can be any distinct amino acid; and
wherein the C-terminus of the polypeptide has less than or equal to 100 amino acids starting from the first amino acid of the GDP-fucose binding domain;
b) contacting the polypeptide with α-1,3-fucosyltransferase activity of step a) with a mixture comprising GDP-fucose as donor substrate, and lactose as acceptor substrate, under conditions where the polypeptide catalyzes the transfer of a fucose residue from the donor substrate to the acceptor substrate, thereby producing α-1,3-fucosyllactose.

These newly identified polypeptides comprising both (or all of SEQ ID NOS: 33 to 36, as the case may be) of the above domains provide for an alternative α-1,3-fucosyltransferase having the ability to use lactose as acceptor substrate over the presently known α-1,3-fucosyltransferases. Polypeptides comprising both (or all of SEQ ID NOS: 33 to 36, as the case may be) of the above domains provide for transferases with similar or higher lactose binding and/or similar or higher transferase properties than presently known α-1,3-fucosyltransferases.

In a first preferred embodiment of the disclosure, a polypeptide useful in the disclosure comprises both (or all of SEQ ID NOS: 33 to 36, as the case may be) of the domains with SEQ ID NOS: 33 to 34 or 36 and wherein SEQ ID NO: 33 is a conserved domain with amino acid sequence YXTEK (SEQ ID NO: 37), wherein X can be any distinct amino acid.

In a second preferred embodiment of the disclosure, a polypeptide useful in the disclosure comprises both (or all of SEQ ID NOS: 33 to 36, as the case may be) of the domains with SEQ ID NOS: 33 to 34 or 36 and wherein SEQ ID NO: 34 is a conserved domain with amino acid sequence [K/D]LX[I/L/M]G[F/Y] (SEQ ID NO: 38), [K/D][L/K]xL[S/G][F/Y] (SEQ ID NO: 39), or [K/D]LXLG[F/Y] (SEQ ID NO: 40), wherein X can be any distinct amino acid.

A further advantage of using some of the polypeptides newly identified to have the ability to use lactose as acceptor substrate and having α-1,3-fucosyltransferase activity and with the newly identified domains resides in the fact that 3-fucosyllactose is produced with a higher purity, than the purity obtained with a reference prior art polypeptide with SEQ ID NO: 18, at the end of reaction or fermentation due to a better conversion ability of the newly identified 3-fucosyltransferases to use lactose for 3FL production. More specifically, the lactose concentration to 3-fucosyllactose concentration ratio is smaller than 1:5, preferably smaller than 1:10, more preferably smaller than 1/20, optimally smaller than 1:40. In another preferred embodiment, the 3-fucosyllactose purity is 80% or higher at the end of fermentation.

According to the disclosure, the method for producing α-1,3-fucosyllactose may be performed in a cell-free system or in a system containing cells. The substrates GDP-fucose and lactose are allowed to react with the alphα-1,3-fucosyltransferase polypeptide for a sufficient time and under sufficient conditions to allow formation of the enzymatic product. These conditions will vary depending upon the amounts and purity of the substrate and enzyme, and whether the system is a cell-free or cellular-based system. These variables will be easily adjusted by those skilled in the art.

In cell-free systems, the polypeptide according to the disclosure, the acceptor substrate(s), donor substrate(s) and, as the case may be, other reaction mixture ingredients, including other glycosyltransferases and accessory enzymes are combined by admixture in an aqueous reaction medium for performing the enzymatic reaction. The enzymes can be utilized free in solution, or they can be bound or immobilized to a support such as a polymer and the substrates may be added to the support. The support may be, e.g., packed in a column.

Cell-containing systems or cellular-based systems for the synthesis of 3-fucosyllactose as described herein may include genetically modified host cells. According to one aspect of the disclosure, the polypeptide with α-1,3-fucosyltransferase activity is produced by a cell producing the polypeptide, e.g., a host cell as described herein. According to another aspect of the disclosure, the GDP-fucose and/or lactose is provided by a cell producing the GDP-fucose and/or lactose. The cell can be the host cell that is also producing the α-1,3-fucosyltransferase. Alternatively, the cell can be another cell than the host cell producing the α-1,3-fucosyltransferase, in which case, the skilled person would talk about cell coupling. Such cell producing GDP-fucose can express an enzyme converting, e.g., fucose, which is to be added to the host cell, to GDP-fucose. This enzyme may be, e.g., a bifunctional fucose kinase/fucose-1-phosphate guanylyltransferase, like Fkp from Bacteroides fragilis, or the combination of one separate fucose kinase together with one separate fucose-1-phosphate guanylyltransferase like they are known from several species including Homo sapiens, Sus scrofa and Rattus norvegicus.

In another embodiment, the disclosure relates to a method for producing α-1,3-fucosyllactose, comprising the following steps:
i) providing a cell genetically modified for the production of α-1,3-fucosyllactose, the cell comprising at least one nucleic acid sequence coding for an enzyme for α-1,3-fucosyllactose synthesis, the cell comprising the expression of a polypeptide with α-1,3-fucosyltransferase activity and with the ability to use lactose as acceptor substrate, wherein the polypeptide comprises:
   a) an amino acid sequence encoding a conserved GDP-fucose binding domain [Y/W/L/H/F/M]X[T/S/C][E/Q/D/A][K/R] (SEQ ID NO: 33);
   b) an amino acid sequence encoding a conserved [K/D][L/K/M]XXX[F/Y] domain (SEQ ID NO: 34), and
   c) wherein additionally the conserved motif [N/H]XDPAXLD (SEQ ID NO: 35) is present at the N-terminal region if the domain of b) equals DM[A/S]VSF (SEQ ID NO: 36);
wherein X can be any distinct amino acid; and
wherein the C-terminus of the polypeptide has less than or equal to 100 amino acids starting from the first amino acid of the GDP-fucose binding domain, and
ii) cultivating the cell in a medium under conditions permissive for the production of α-1,3-fucosyllactose, thereby producing 3-FL.

In a further embodiment, the disclosure relates to a method for producing α-1,3-fucosyllactose the method comprising the steps of:
a) providing a host cell expressing the polypeptide with α-1,3-fucosyltransferase activity and with the ability to use lactose as acceptor substrate, as defined herein;
b) growing, under suitable nutrient conditions permissive for the production of the α-1,3-fucosyllactose, and permissive for the expression of the polypeptide with α-1,3-fucosyltransferase activity, the host cell;
c) providing, simultaneously or subsequently to step b), a donor substrate GDP-fucose and the acceptor substrate lactose, in order for the α-1,3-fucosyltransferase polypeptide to catalyze the transfer of a fucose residue from GDP-fucose to lactose, thereby producing α-1,3-fucosyllactose. Optionally the produced 3FL is then separated from the host cell and/or the medium of its growth.

According to yet another embodiment, the production of the 3-fucosyllactose in the methods as described herein is performed by means of a heterologous or homologous (over)expression of the polynucleotide encoding the α-1,3-fucosyltransferase by the cell.

In the methods of the disclosure as described herein, the host cell can be transformed or transfected to express an exogenous polypeptide as described herein and with α-1,3-fucosyltransferase activity and with the ability to use lactose as an acceptor substrate. As such, the disclosure relates to a method for producing α-1,3-fucosyllactose using a host cell, comprising the following steps:
a) growing, a host cell transformed or transfected to express an exogenous polypeptide with α-1,3-fucosyltransferase activity and with the ability to use lactose as an acceptor substrate, wherein the polypeptide is set forth herein; and
b) providing, simultaneously or subsequently to step a), a donor substrate GDP-fucose and an acceptor substrate lactose, wherein the α-1,3-fucosyltransferase polypeptide catalyzes the transfer of a fucose residue from the donor substrate to the acceptor substrate, thereby producing α-1,3-fucosyllactose.

Preferably, the exogenous polypeptide with α-1,3-fucosyltransferase activity and with the ability to use lactose as an acceptor substrate as used herein, produces 3FL with a lactose concentration to 3FL concentration ratio at the end of fermentation smaller than 1:5.

The ratio concentration lactose to concentration 3FL can be less than 1:5, 1:6, 1:7, 1:8, 1:9, 1:10, 1:11, 1:12, 1:13, 1:14, 1:15, 1:16, 1:17, 1:18, 1:19, 1:20, 1:21, 1:22, 1:23, 1:24, 1:25, 1:26, 1:27, 1:28, 1:29, 1:30, 1:31, 1:32, 1:33, 1:34, 1:35, 1:36, 1:37, 1:38, 1:39, 1:40, 1:45, 1:50, 1:55, 1:60, 1:65, 1:70, 1:75, 1:80, 1:85, 1:90, 1:95, 1:100, 1:110, 1:120, 1:130, 1:140, 1:150, 1:160, 1:170, 1:180; 1:190, 1:200, 1:300, 1:400, 1:500, 1:600, 1:700, 1:800, 1:900, 1:1000.

In a preferred embodiment, the ratio lactose concentration on 3FL concentration oflower than 1:5, 1:6, 1:7, 1:8, 1:9, 1:10, 1:11, 1:12, 1:13, 1:14, 1:15, 1:16, 1:17, 1:18, 1:19, 1:20, 1:21, 1:22, 1:23, 1:24, 1:25, 1:26, 1:27, 1:28, 1:29, 1:30, 1:31, 1:32, 1:33, 1:34, 1:35, 1:36, 1:37, 1:38, 1:39, 1:40, 1:45, 1:50, 1:55, 1:60, 1:65, 1:70, 1:75, 1:80, 1:85, 1:90, 1:95, 1:100, 1:110, 1:120, 1:130, 1:140, 1:150, 1:160, 1:170, 1:180; 1:190, 1:200, 1:300, 1:400, 1:500, 1:600, 1:700, 1:800, 1:900, 1:1000 is obtained within a production process resulting in a final lactose concentration of lower than 25 g/L, 20 g/L, 15 g/L 10 g/L, 9 g/L, 8 g/L, 7 g/L, 6 g/L, 5 g/L, 4 g/L, 3 g/L, 2 g/L, 1 g/L, 0.5 g/L, 0.25 g/L, 0.1 g/L or 0 g/L.

In another embodiment, the ratio lactose concentration on 3FL concentration of lower than 1:5, 1:6, 1:7, 1:8, 1:9, 1:10, 1:11, 1:12, 1:13, 1:14, 1:15, 1:16, 1:17, 1:18, 1:19, 1:20, 1:21, 1:22, 1:23, 1:24, 1:25, 1:26, 1:27, 1:28, 1:29, 1:30, 1:31, 1:32, 1:33, 1:34, 1:35, 1:36, 1:37, 1:38, 1:39, 1:40, 1:45, 1:50, 1:55, 1:60, 1:65, 1:70, 1:75, 1:80, 1:85, 1:90, 1:95, 1:100, 1:110, 1:120, 1:130, 1:140, 1:150, 1:160, 1:170, 1:180; 1:190, 1:200, 1:300, 1:400, 1:500, 1:600, 1:700, 1:800, 1:900, 1:1000 is obtained within a production process wherein the lactose concentration is fed at substrate limiting conditions, wherein the substrate limitation is defined as the concentration in the bioreactor that determines the rate of conversion of the substrate.

In another embodiment, the ratio lactose concentration on 3FL concentration of lower than 1:5, 1:6, 1:7, 1:8, 1:9, 1:10, 1:11, 1:12, 1:13, 1:14, 1:15, 1:16, 1:17, 1:18, 1:19, 1:20, 1:21, 1:22, 1:23, 1:24, 1:25, 1:26, 1:27, 1:28, 1:29, 1:30, 1:31, 1:32, 1:33, 1:34, 1:35, 1:36, 1:37, 1:38, 1:39, 1:40, 1:45, 1:50, 1:55, 1:60, 1:65, 1:70, 1:75, 1:80, 1:85, 1:90, 1:95, 1:100, 1:110, 1:120, 1:130, 1:140, 1:150, 1:160, 1:170, 1:180; 1:190, 1:200, 1:300, 1:400, 1:500, 1:600, 1:700, 1:800, 1:900, 1:1000 is obtained within a production process wherein the lactose is formed in the cell at rate limiting conditions.

In another embodiment, the 3-fucosyllactose purity in the broth is higher than about 80%, such as 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, 99.5%, 99.6%, 99.7%, 99.8%, 99.9% on sum of (lactose and 3FL) in broth. As used herein, the 3-fucosyllactose purity is defined as the ratio of the 3FL concentration to the sum of the 3FL concentration and the lactose concentration ([3FL]/([3FL]+[lactose])).

According to the disclosure, the GDP-fucose and/or lactose can be fed to the host cell in the fermentation medium or aqueous culture medium. Alternatively, the GDP-fucose and/or lactose can be provided by an enzyme simultaneously expressed in the host cell or by the metabolism of the host cell. Accordingly, the host cell will also produce the α-1,3-fucosyltransferase next to the GDP-fucose and/or lactose. In another embodiment, the GDP-fucose and/or lactose can be produced by a cell that is another cell other than the host cell producing the α-1,3-fucosyltransferase, in which case, the skilled person would talk about cell coupling. Such cell producing GDP-fucose can express an enzyme converting, e.g., fucose, which is to be added to the host cell, to GDP-fucose. This enzyme may be, e.g., a bifunctional fucose kinase/fucose-1-phosphate guanylyltransferase, like Fkp from Bacteroides fragilis, or the combination of one separate fucose kinase together with one separate fucose-1-phosphate guanylyltransferase like they are known from several species including Homo sapiens, Sus scrofa and Rattus norvegicus.

According to yet another embodiment, the production of the α-1,3-fucosyllactose is performed by means a host cell as described herein comprising a heterologous or homologous (over)expression of the polynucleotide encoding the α-1,3-fucosyltransferase.

In a further aspect, the present disclosure provides for a method for producing α-1,3-fucosyllactose as described herein, wherein the method further comprises a step of separating the α-1,3-fucosyllactose from the host cell or the medium of its growth.

As used herein, the term “separating” means harvesting, collecting or retrieving from the reaction mixture and/or from the cell producing the α-1,3-fucosyltransferase, the α-1,3-fucosyllactose produced by the α-1,3-fucosyltransferase according to the disclosure.

In case α-1,3-fucosyllactose is made by use of cells or fermentation, the 3-FL can be separated in a conventional manner from the aqueous culture medium, in which the mixture was made. In case the alphα-1,3-fucosyllactose is still present in the cells producing the α-1,3-fucosyllactose, conventional manners to free or to extract the α-1,3-fucosyllactose out of the cells can be used, such as cell destruction using high pH, heat shock, sonication, French press, homogenization, enzymatic hydrolysis, chemical hydrolysis, solvent hydrolysis, detergent, hydrolysis, etc. The culture medium, reaction mixture and/or cell extract, together and separately called 3-FL containing mixture, can then be further used for separating the 3-FL. This preferably involves clarifying the 3-FL containing mixtures to remove suspended particulates and contaminants, particularly cells, cell components, insoluble metabolites and debris produced by culturing the genetically modified cell and/or performing the enzymatic reaction. In this step, the 3-FL containing mixture can be clarified in a conventional manner. Preferably, the 3-FL containing mixture is clarified by centrifugation, flocculation, decantation and/or filtration. A second step of separating the 3-FL from the 3-FL containing mixture preferably involves removing substantially all the proteins, as well as peptides, amino acids, RNA and DNA and any endotoxins and glycolipids that could interfere with the subsequent separation step, from the 3-FL containing mixture, preferably after it has been clarified. In this step, proteins and related impurities can be removed from the 3-FL containing mixture in a conventional manner. Preferably, proteins, salts, byproducts, color and other related impurities are removed from the 3-FL containing mixture by ultrafiltration, nanofiltration, reverse osmosis, microfiltration, activated charcoal or carbon treatment, tangential flow high-performance filtration, tangential flow ultrafiltration, affinity chromatography, ion exchange chromatography (such as but not limited to cation exchange, anion exchange, mixed bed ion exchange), hydrophobic interaction chromatography and/or gel filtration (i.e., size exclusion chromatography), particularly by chromatography, more particularly by ion exchange chromatography or hydrophobic interaction chromatography or ligand exchange chromatography. With the exception of size exclusion chromatography, proteins and related impurities are retained by a chromatography medium or a selected membrane, while 3-FL remains in the 3-FL containing mixture.

3-FL is further separated from the reaction mixture and/or culture medium and/or cell with or without further purification steps by evaporation, lyophilization, crystallization, precipitation, and/or drying, spray drying.

In an even further aspect, the present disclosure also provides for a further purification of the α-1,3-fucosyllactose. A further purification of the alphα-1,3-fucosyllactose may be accomplished, for example, by use of (activated) charcoal or carbon, nanofiltration, ultrafiltration or ion exchange to remove any remaining DNA, protein, LPS, endotoxins, or other impurity. Alcohols, such as ethanol, and aqueous alcohol mixtures can also be used. Another purification step is accomplished by crystallization, evaporation or precipitation of the product. Another purification step is to dry, spray dry or lyophilize α-1,3-fucosyllactose.

The separated and preferably also purified 3-FL can be used as a supplement in infant formulas and for treating various diseases in newborn infants.

Another aspect of the disclosure provides for a method wherein the polypeptide and preferably also the 3-FL is produced in and/or by a fungal, yeast, bacterial, insect, animal and plant expression system or cell as described herein. The expression system or cell is chosen from the list comprising a bacterium, a yeast, or a fungus, or, refers to a plant or animal cell. The latter bacterium preferably belongs to the phylum of the Proteobacteria or the phylum of the Firmicutes or the phylum of the Cyanobacteria or the phylum Deinococcus-Thermus. The latter bacterium belonging to the phylum Proteobacteria belongs preferably to the family Enterobacteriaceae, preferably to the species Escherichia coli. The latter bacterium preferably relates to any strain belonging to the species Escherichia coli such as but not limited to Escherichia coli B, Escherichia coli C, Escherichia coli W, Escherichia coli K12, Escherichia coli Nissle. More specifically, the latter term relates to cultivated Escherichia coli strains—designated as E. coli K12 strains—which are well-adapted to the laboratory environment, and, unlike wild type strains, have lost their ability to thrive in the intestine. Well-known examples of the E. coli K12 strains are K12 Wild type, W3110, MG1655, M182, MC1000, MC1060, MC1061, MC4100, JM101, NZN111 and AA200. Hence, the disclosure specifically relates to a mutated and/or transformed Escherichia coli host cell or strain as indicated above wherein the E. coli strain is a K12 strain. More preferably, the Escherichia coli K12 strain is E. coli MG1655. The latter bacterium belonging to the phylum Firmicutes belongs preferably to the Bacilli, preferably Lactobacillales, with members such as Lactobacillus lactis, Leuconostoc mesenteroides, or Bacillales with members such as from the genus Bacillus, such as Bacillus subtilis or, B. amyloliquefaciens. The latter Bacterium belonging to the phylum Actinobacteria, preferably belonging to the family of the Corynebacteriaceae, with members Corynebacterium glutamicum or C. afermentans, or belonging to the family of the Streptomycetaceae with members Streptomyces griseus or S. fradiae. The latter yeast preferably belongs to the phylum of the Ascomycota or the phylum of the Basidiomycota or the phylum of the Deuteromycota or the phylum of the Zygomycetes. The latter yeast belongs preferably to the genus Saccharomyces, Pichia, Komagataella, Hansenula, Kluyveromyces, Yarrowia or Starmerella. The latter fungus belongs preferably to the genus Rhizopus, Dictyostelium, Penicillium, Mucor or Aspergillus.

According to a further aspect of the disclosure, the polynucleotide encoding the polypeptide with alphα-1,3-fucosyltransferase activity is adapted to the codon usage of the respective cell or expression system.

In a further preferred embodiment, the method of the disclosure uses a culture medium for growth of the host cell or microorganism comprising the alphα-1,3-fucosyltransferase of the disclosure, wherein the lactose concentration in the culture medium ranges from 50 to 150 g/L. Such lactose concentration in the culture medium can be 50 g/L, 55 g/L, 60 g/L, 65 g/L, 70 g/L, 75 g/L, 80 g/L, 85 g/L, 90 g/L, 95 g/L, 100 g/L, 105 g/L, 110 g/L, 115 g/L, 120 g/L, 125 g/L, 130 g/L, 135 g/L, 140 g/L, 145 g/L, or 150 g/L.

In a further preferred embodiment, the method of the disclosure produces a final concentration of 3-fucosyllactose ranges between 70 g/L to 200 g/L. Such 3-FL concentration being 70 g/L, 75 g/L, 80 g/L, 85 g/L, 90 g/L, 95 g/L, 100 g/L, 105 g/L, 110 g/L, 115 g/L, 120 g/L, 125 g/L, 130 g/L, 135 g/L, 140 g/L, 145 g/L, 145 g/L, 150 g/L, 155 g/L, 160 g/L, 165 g/L, 170 g/L, 175 g/L, 180 g/L, 185 g/L, 190 g/L, 195 g/L, or 200 g/L. Higher lactose concentrations in the culture medium can provide even higher 3-FL final concentrations obtained in the production method.

In a further preferred embodiment, the method of the disclosure produces a final concentration of 3FL ranging between 70 g/L to 200 g/L as explained above, and wherein the 3FL purity in the broth is 80% or more. The 3FL purity according to the disclosure is at least about 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, 99.5%, 99.6%, 99.7%, 99.8%, 99.9%.

In the methods of the disclosure as described herein the polypeptide with α-1,3-fucosyltransferase activity and with the ability to use lactose as acceptor substrate comprises:
a) an amino acid sequence encoding a conserved GDP-fucose binding domain [Y/W/L/H/F/M]X[T/S/C][E/Q/D/A][K/R] (SEQ ID NO: 33);
b) an amino acid sequence encoding a conserved [K/D][L/K/M]XXX[F/Y] domain (SEQ ID NO: 34), and
c) wherein additionally the conserved motif [N/H]XDPAXLD (SEQ ID NO: 35) is present at the N-terminal region if the domain of b) equals DM[A/S]VSF (SEQ ID NO: 36);
wherein X can be any distinct amino acid; and
wherein the C-terminus of the polypeptide has less than or equal to 100 amino acids starting from the first amino acid of the GDP-fucose binding domain.

Within the scope of the disclosure, such polypeptide proved to have lactose binding α-1,3-fucosyltransferase activity and preferably has better lactose conversion efficiency compared to the presently known α-1,3-fucosyltransferase enzymes.

In a preferred embodiment of the disclosure, the polypeptide with α-1,3-fucosyltransferase activity and with the ability to use lactose as acceptor substrate comprises an amino acid sequence selected from the group consisting of:
i) any one of SEQ ID NOS: 2, 4, 6, 8, 10, 12, 14, 16, 20, 22, 28, 30 or 32 of the attached sequence listing;
ii) an amino acid sequence having 87% or more sequence identity to the full length amino acid sequence of SEQ ID NOS: 2, 20 or 22;
iii) an amino acid sequence having 80% or more sequence identity to the full length amino acid sequence of any one of SEQ ID NOS: 6, 8, 10, 12, 14, 16, 28, 30 or 32;
iv) a fragment of an amino acid sequence shown in SEQ ID NOS: 2, 20 or 22, wherein the fragment comprises at least 45 contiguous amino acids thereof;
v) a fragment of an amino acid sequence shown in any one of SEQ ID NOS: 6, 8, 10, 12, 14, 16, 28, 30 or 32, wherein the fragment comprises at least 10 contiguous amino acids thereof and has lactose binding alphα-1,3-fucosyltransferase activity.

Optionally, the polypeptide is further modified by an N-terminal and/or C-terminal amino acid stretch.

The amino acid sequence of the polypeptide used herein can be a sequence chosen from SEQ ID NOS: 2, 4, 6, 8, 10, 12, 14, 16, 20, 22, 28, 30 or 32 of the attached sequence listing. The amino acid sequence can also be an amino acid sequence that has greater than about 87% sequence identity, 87%, 88%, 89%, 90%, 91%, 92%, 93%, 94%, 95%, 95.5%, 96%, 96.5%, 97%, 97.5%, 98%, 98.5%, 99%, 99.5%, 99.6%, 99.7%, 99.8%, 99.9% sequence identity to the full length amino acid sequence of any one of SEQ ID NOS: 2, 20 or 22. The amino acid sequence can also be an amino acid sequence that has greater than about 80% sequence identity, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, 99.5%, 99.6%, 99.7%, 99.8%, 99.9% sequence identity to the full length amino acid sequence of any one of SEQ ID NOS: 6, 8, 10, 12, 14, 16, 28, 30 or 32.

Furthermore, within the scope of the disclosure, the amino acid sequence can be a fragment of an amino acid sequence shown in any one of SEQ ID NOS: 2, 20 or 22, wherein the fragment comprises at least 45 contiguous amino acids thereof; alternatively the amino acid sequence can be a fragment of an amino acid sequence shown in any one of SEQ ID NOS: 6, 8, 10, 12, 14, 16, 28, 30 or 32, wherein the fragment comprises at least 10 contiguous amino acids thereof and has lactose binding α-1,3-fucosyltransferase activity.

Further included in the scope of the disclosure, is an α-1,3-fucosyltransferase polypeptide as described herein that is optionally further modified by an N-terminal and/or C-terminal amino acid stretch. Such amino acid stretch is to be understood as an addition of polypeptide sequences at the N-terminus and/or C-terminus of the polypeptide. For example, polypeptide sequences may be fused to the alphα-1,3-fucosyltransferase polypeptide in order to effectuate additional enzymatic activity. Such amino acid stretch can be a specific tag and/or HQ-tag; an extension of up to 20 amino acids, e.g., 1, 2, 3, 4, 5, 6, 7, 8, 9, 10, 11, 12, 13, 14, 15, 16, 17, 18, 19 or 20 amino acids; such extension can also be 25, 30, 35, 40, 45, 50, 55, 60, 65, 70 or more amino acids long. The optional N-terminal and/or C-terminal amino acid stretch can also be a tag for purification, a tag for increasing the solubility of the polypeptide, a tag or amino acid stretch for metabolon formation, a tag for protein metabolomics, a tag for substrate binding, another polypeptide with the same or a different function in a gene fusion, such as but not limited to a polypeptide coding for GDP-fucose synthase, galactosyltransferase, fucosyltransferase, bifunctional fucose kinase/fucose-1-phosphate guanylyltransferase or fucose-1-phosphate guanylyltransferase, wherein the other polypeptide is optionally fused to the alphα-1,3-fucosyltransferase polypeptide via a peptide linker. For example, the alphα-1,3-fucosyltransferase polypeptide as described herein optionally includes one or more exogenous affinity tags, e.g., purification or substrate binding tags, such as a 6 His tag sequence, a GST tag, a HQ tag, an HA tag sequence, a plurality of 6 His tag sequences, a plurality of GST tags, a plurality of HA tag sequences, a SNAP-tag, a SUMOstar tag. Other examples include proteolytic cleavage sites, retention sites, cleavage sites, polyhistidine tags, biotin, avidin, BiTag sequences, S tags, enterokinase sites, thrombin sites, antibodies or antibody domains, antibody fragments, antigens, receptors, receptor domains, receptor fragments, ligands, dyes, acceptors, quenchers, or combinations thereof.

In addition, α-1,3-fucosyltransferase polypeptides may include proteins or polypeptides that represent functionally equivalent polypeptides. Such an equivalent α-1,3-fucosyltransferase polypeptide may contain deletions, additions or substitutions of amino acid residues within the amino acid sequence encoded by the α-1,3-fucosyltransferase polynucleotides described herein, but that results in a silent change, thus producing a functionally equivalent α-1,3-fucosyltransferase. Amino acid substitutions may be made on the basis of similarity in polarity, charge, solubility, hydrophobicity, hydrophilicity, and/or the amphipathic nature of the residues involved. For example, nonpolar (hydrophobic) amino acids include alanine, leucine, isoleucine, valine, proline, phenylalanine, tryptophan, and methionine; planar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid. Within the context of this disclosure, “functionally equivalent,” as used herein, refers to a polypeptide capable of exhibiting a substantially similar in vivo activity as the lactose binding α-1,3-fucosyltransferase polypeptides of the present disclosure as judged by any of a number of criteria, including but not limited to enzymatic activity.

Included within the scope of the disclosure are alphα-1,3-fucosyltransferase proteins, polypeptides, and derivatives (including fragments) that are differentially modified during or after translation. Furthermore, non-classical amino acids or chemical amino acid analogues can be introduced as a substitution or addition into the alphα-1,3-fucosyltransferase polypeptide sequence.

The α-1,3-fucosyltransferase polypeptide may be produced by expression by polynucleotides produced via recombinant DNA technology using techniques well known in the art. Methods that are well known to those skilled in the art can be used to construct expression vectors containing α-1,3-fucosyltransferase coding sequences and appropriate transcriptional and/or translational control signals. These methods include, for example, in vitro recombinant DNA techniques, synthetic techniques, and in vivo genetic recombination. See, for example, the techniques described in Sambrook et al. (2001) Molecular Cloning: a laboratory manual, 3rd Edition, Cold Spring Harbor Laboratory Press, CSH, New York or to Current Protocols in Molecular Biology, John Wiley and Sons, N.Y. (1989 and yearly updates). Alternatively, the α-1,3-fucosyltransferase polypeptide may be produced by direct synthesis, by extraction of the cell that produces the polypeptide in nature or within a cell free and/or in vitro system.

The suitability of the newly identified alphα-1,3-fucosyltransferases having the ability to bind lactose to be used for producing 3-fucosyllactose, and preferably producing such 3FL with a purity of 80% or more, is highly surprising, and, thus, their use represents an excellent tool to easily, efficiently and cost-effectively produce 3-fucosyllactose.

The polynucleotide encoding the α-1,3-fucosyltransferase polypeptide may be produced via recombinant DNA technology using techniques well known in the art. Methods that are well known to those skilled in the art can be used to construct expression vectors containing α-1,3-fucosyltransferase coding sequences and appropriate transcriptional and/or translational control signals. These methods include, for example, in vitro recombinant DNA techniques, synthetic techniques, and in vivo genetic recombination. See, for example, the techniques described in Sambrook et al. (2001) Molecular Cloning: a laboratory manual, 3rd Edition, Cold Spring Harbor Laboratory Press, CSH, New York or to Current Protocols in Molecular Biology, John Wiley and Sons, N.Y. (1989 and yearly updates).

According to another aspect of the disclosure, a vector is provided, containing a polynucleotide encoding a polypeptide with alphα-1,3-fucosyltransferase activity as described herein, wherein the polynucleotide is operably linked to control sequences recognized by a host cell transformed with the vector. In a particularly preferred embodiment, the vector is an expression vector, and, according to another aspect of the disclosure, the vector can be present in the form of a plasmid, cosmid, phage, liposome, or virus.

Thus, the polynucleotide according to the disclosure may, e.g., be comprised in a vector that is to be stably transformed/transfected into host cells. In the vector, the polynucleotide of the disclosure is under control of a promoter. The promoter can be, e.g., an inducible promoter, so that the expression of the gene/polynucleotide can be specifically targeted, and, if desired, the gene may be overexpressed in that way. The promoter can also be a constitutive promoter.

A great variety of expression systems can be used to produce the polypeptides of the disclosure. Such vectors include, among others, chromosomal, episomal and virus-derived vectors, e.g., vectors derived from bacterial plasmids, from bacteriophage, from transposons, from yeast episomes, from insertion elements, from yeast chromosomal elements, from viruses, and vectors derived from combinations thereof, such as those derived from plasmid and bacteriophage genetic elements, such as cosmids and phagemids. These vectors may contain selection markers such as but not limited to antibiotic markers, auxotrophic markers, toxin-antitoxin markers, RNA sense/antisense markers. The expression system constructs may contain control regions that regulate as well as engender expression. Generally, any system or vector suitable to maintain, propagate or express polynucleotides and/or to express a polypeptide in a host may be used for expression in this regard. The appropriate DNA sequence may be inserted into the expression system by any of a variety of well-known and routine techniques, such as, for example, those set forth in Sambrook et al., see above.

For recombinant production, host cells can be genetically engineered to incorporate expression systems or portions thereof or polynucleotides of the invention. Introduction of a polynucleotide into the host cell can be effected by methods described in many standard laboratory manuals, such as Davis et al., Basic Methods in Molecular Biology, (1986), and Sambrook et al., 1989, supra.

According to another aspect of the disclosure, a host cell is provided containing the vector as described above.

According to a further aspect, the disclosure provides a host cell genetically modified for the production of α-1,3-fucosyllactose, wherein the host cell comprises at least one nucleic acid sequence coding for an enzyme for 3-fucosyllactose synthesis and wherein the cell comprises the expression of a polypeptide with α-1,3-fucosyltransferase activity and with the ability to use lactose as acceptor substrate and the polypeptide being as described herein.

As used herein, the term “host cell” is presently defined as a cell that has been transformed or transfected or is capable of transformation or transfection by an exogenous polynucleotide sequence, thus containing at least one sequence not naturally occurring in the host cell.

A variety of host-expression vector systems may be utilized to express the alpha-1,3-fucosyltransferase polynucleotides of the disclosure. Such host-expression systems represent vehicles by which the coding sequences of interest may be produced and subsequently purified, but also represent cells that, when transformed or transfected with the appropriate nucleotide coding sequences, exhibit the alphα-1,3-fucosyltransferase gene product of the disclosure in situ.

According to another aspect of the disclosure, a host cell for the production of 3-fucosyllactose is provided wherein the host cell contains a sequence consisting of a polynucleotide encoding a polypeptide with lactose binding alphα-1,3-fucosyltransferase activity as described herein, wherein the sequence is a sequence foreign to the host cell and wherein the sequence is integrated in the genome of the host cell. The polynucleotide is operably linked to control sequences recognized by the host cell.

According to an alternative aspect of the disclosure, a host cell for the production of 3-fucosyllactose is provided wherein the host cell contains a vector comprising the polynucleotide described herein, wherein the polynucleotide being operably linked to control sequences recognized by a host cell transformed with the vector.

In a further aspect, the present disclosure also provides for a method for the production of α-1,3-fucosyllactose, comprising the steps of:
a) providing a cell as described herein, and
b) cultivating the cell in a medium under conditions permissive for the production of α-1,3-fucosyltransferase.

Preferably, the α-1,3-fucosyltransferase is separated from the cultivation as described herein. Preferably, also a purification can be done as described herein.

In another further aspect, the disclosure provides for use of the cell as described herein for the production of 3-fucosyllactose.

According to a further aspect of the disclosure, a microorganism is provided expressing the alphα-1,3-fucosyltransferase as described herein and preferably encoded by the polynucleotide as described herein.

The term micro-organism or organism or cell or host cell as used herein refers to a microorganism chosen from the list comprising a bacterium, a yeast, or a fungus, or, refers to a plant or animal cell. The latter bacterium preferably belongs to the phylum of the Proteobacteria or the phylum of the Firmicutes or the phylum of the Cyanobacteria or the phylum Deinococcus-Thermus. The latter bacterium belonging to the phylum Proteobacteria belongs preferably to the family Enterobacteriaceae, preferably to the species Escherichia coli. The latter bacterium preferably relates to any strain belonging to the species Escherichia coli such as but not limited to Escherichia coli B, Escherichia coli C, Escherichia coli W, Escherichia coli K12, Escherichia coli Nissle. More specifically, the latter term relates to cultivated Escherichia coli strains—designated as E. coli K12 strains—that are well-adapted to the laboratory environment, and, unlike wild type strains, have lost their ability to thrive in the intestine. Well-known examples of the E. coli K12 strains are K12 Wild type, W3110, MG1655, M182, MC1000, MC1060, MC1061, MC4100, JM101, NZN111 and AA200. Hence, the present disclosure specifically relates to a mutated and/or transformed Escherichia coli host cell or strain as indicated above wherein the E. coli strain is a K12 strain. More preferably, the Escherichia coli K12 strain is E. coli MG1655. The latter bacterium belonging to the phylum Firmicutes belongs preferably to the Bacilli, preferably Lactobacillales, with members such as Lactobacillus lactis, Leuconostoc mesenteroides, or Bacillales with members such as from the genus Bacillus such as Bacillus subtilis or B. amyloliquefaciens. The latter Bacterium belonging to the phylum Actinobacteria, preferably belonging to the family of the Corynebacteriaceae, with members Corynebacterium glutamicum or C. afermentans, or belonging to the family of the Streptomycetaceae with members Streptomyces griseus or S. fradiae. The latter yeast preferably belongs to the phylum of the Ascomycota or the phylum of the Basidiomycota or the phylum of the Deuteromycota or the phylum of the Zygomycetes. The latter yeast belongs preferably to the genus Saccharomyces, Pichia, Komagataella, Hansenula, Kluyveromyces, Yarrowia or Starmerella. The latter fungus belongs preferably to the genus Rhizopus, Dictyostelium, Penicillium, Mucor or Aspergillus.

According to another aspect of the disclosure, the polynucleotide encoding the polypeptide with lactose binding alphα-1,3-fucosyltransferase activity is adapted to the codon usage of the respective host cell.

A further aspect of the disclosure provides for the use of a polypeptide as described herein for the production of alphα-1,3-fucosyllactose. A further aspect of the disclosure provides for the use of a polynucleotide as described herein or of the vector as described herein, for the production of alphα-1,3-fucosyllactose.

According to one other embodiment, there is provided hitherto unknown lactose binding alphα-1,3-fucosyltransferases. The disclosure provides an isolated and/or synthesized polypeptide with a lactose binding alphα-1,3-fucosyltransferase activity wherein the polypeptide comprises:
an amino acid sequence encoding a conserved GDP-fucose binding domain [Y/W/L/H/F/M]X[T/S/C][E/Q/D/A][K/R] (SEQ ID NO: 33) and a conserved [K/D][L/K/M]XXX[F/Y] domain (SEQ ID NO: 34), where additionally the conserved motif [N/H]XDPAXLD (SEQ ID NO: 35) is present at the N-terminal region if this domain equals DM[A/S]VSF (SEQ ID NO: 36), wherein X can be any distinct amino acid, and the C-terminus of the amino acid sequence having less than or equal to 100 amino acids starting from the first amino acid of the GDP-fucose binding domain.

Preferably, the polypeptide is selected from the group consisting of:
i) SEQ ID NOS: 2, 4, 6, 8, 10, 12, 14, 16, 20, 22, 28, 30 or 32 of the attached sequence listing;
ii) an amino acid sequence having 87% or more sequence identity to the full length of SEQ ID NOS: 2, 20 or 22;
iii) an amino acid sequence comprising at least 80% sequence identity to the full-length amino acid sequence of any one of SEQ ID NOS: 6, 8, 10, 12, 14, 16, 28, 30 or 32;
iv) a fragment of an amino acid sequence shown in any one of SEQ ID NOS: 2, 20 or 22, wherein the fragment comprises at least 45 contiguous amino acids thereof;
v) a fragment of an amino acid sequence shown in any one of SEQ ID NOS: 6, 8, 10, 12, 14, 16, 28, 30 or 32, wherein the fragment comprises at least 10 contiguous amino acids thereof.

Optionally, the polypeptide is further modified by an N-terminal and/or C-terminal amino acid stretch.

Within the scope of the disclosure, the isolated and/or synthesized polypeptide has lactose binding alphα-1,3-fucosyltransferase activity. Such polypeptide comprises an amino acid sequence encoding a conserved GDP-fucose binding domain [Y/W/L/H/F/M]X[T/S/C] [E/Q/D/A] [K/R] (SEQ ID NO: 33) and a conserved [K/D][L/K/M]XXX[F/Y] domain (SEQ ID NO: 34), where additionally the conserved motif [N/H]XDPAXLD (SEQ ID NO: 35) is present at the N-terminal region if this domain equals DM[A/S]VSF (SEQ ID NO: 36), wherein X can be any distinct amino acid, and the C-terminus of the amino acid sequence having less than or equal to 100 amino acids, such as 100, 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40 amino acids, starting from the first amino acid of the above defined conserved GDP-fucose binding domain.

Further included in the scope of the disclosure is an alpha 1,3-fucosyltransferase polypeptide as described herein that is optionally further modified by an N-terminal and/or C-terminal amino acid stretch.

The newly identified lactose binding alphα-1,3-fucosyltransferases were surprisingly found to be useable to perform reactions that are not naturally occurring. Furthermore, it has been found that the above identified alphα-1,3-fucosyltransferases are able to use lactose as substrate with similar or higher lactose binding properties than the presently known alphα-1,3-fucosyltransferase enzymes and are able to produce 3-fucosyllactose.

Up to the present day, the newly identified fucosyltransferases of the disclosure were not described to have lactose binding alphα-1,3-fucosyltransferase activity, as can be seen in Table 1.

TABLE 1

| SEQ ID NO: | DNA Identifier | NCBI_name | Organism |
|---|---|---|---|
| SEQ ID NOS: 1-2 | SMF69967.1 | Glycosyltransferase family 10 | Azospirillum oryzae A2P |
| | | (fucosyltransferase) C-term | |
| | | [Azospirillum oryzae] | |
| SEQ ID NOS: 3-4 | WP_042442472.1 | putative glycosyltransferase | Azospirillum lipoferum |
| | | [Azospirillum lipoferum 4B] | |
| SEQ ID NOS: 5-6 | AIL32582.1 | hypothetical protein IX83_03990 | Basilea psittacipulmonis |
| | | [Basilea psittacipulmonis DSM | |
| | | 24701] | |
| SEQ ID NOS: 7-8 | ADG66884.1 | putative LPS biosynthesis related | Planctopirus limnophila |
| | | glycosyltransferase [Planctopirus | (strain ATCC 43296/DSM |
| | | limnophila DSM 3776] | 3776/IFAM 1008/290) |
| | | | (Planctomyces limnophilus) |
| SEQ ID NOS: 9-10 | KHJ37904.1 | glycosyltransferase family 10 | Pedobacter glucosidilyticus |
| | | (fucosyltransferase) [Pedobacter | |
| | | glucosidilyticus] | |
| SEQ ID NOS: 11-12 | WP_081748371.1 | hypothetical protein [Porphyromonas | Porphyromonas catoniae |
| | | catoniae] | (WGS, in genbank: |
| | | | JDFF01000001 till |
| | | | JDFF010000025) |
| SEQ ID NOS: 13-14 | WP_052080772.1 | hypothetical protein [Porphyromonas | Porphyromonas sp. COT- |
| | | sp. COT-239 OH1446] | 239 OH1446 (contig_18; |
| | | | NZJRAO01000018.1) |
| SEQ ID NOS: 15-16 | EHG19535.1 | hypothetical protein | Selenomonas infelix ATCC |
| | | HMPREF9334_01850 [Selenomonas | 43532 |
| | | infelix ATCC 43532] | |
| SEQ ID NOS: 19-20 | WP_109445332 | alpha-1,3-fucosyltransferase | Azospirillum sp. TSH64 |
| | | [Azospirillum sp. TSH64] | |
| SEQ ID NOS: 21-22 | QCG87584.1 | alpha-1,3-fucosyltransferase | Azospirillum sp. TSH100 |
| | | [Azospirillum sp. TSH100] | |
| SEQ ID NOS: 27-28 | SEQ36152.1 | Glycosyltransferase family 10 | Butyrivibrio sp. TB |
| | | (fucosyltransferase) C-term | |
| | | [Butyrivibrio sp. TB] | |
| SEQ ID NOS: 29-30 | EKX99948.1 | hypothetical protein | Porphyromonas catoniae |
| | | HMPREF9134_01857 | F0037 |
| | | [Porphyromonas catoniae F0037] | |
| SEQ ID NOS: 31-32 | SHI32494.1 | Glycosyltransferase family 10 | Butyrivibrio fibrisolvens |
| | | (fucosyltransferase) C-term | |
| | | [Butyrivibrio fibrisolvens | |

As shown in Table 2, it was also found that the newly identified α-1,3-fucosyltransferases having the ability to bind lactose to be used for producing 3-fucosyllactose, all shared the same special feature of having an amino acid sequence comprising a conserved GDP-fucose binding domain [Y/W/L/H/F/M]X[T/S/C][E/Q/D/A][K/R] (SEQ ID NO: 33) wherein x can be any distinct amino acid and wherein the C-terminus of the amino acid sequence has less than or equal to 100 amino acids starting from the first amino acid of the GDP-fucose binding domain. This in contrast with the known lactose binding 3-fucosyltransferases as described in, e.g., WO2012/049083 having a C-terminus that is longer than 100 amino acids starting from the first amino acid of the above defined GDP-fucose binding domain.

TABLE 2

| | | | C-terminus length |
|---|---|---|---|
| | | | from GDP-fucose |
| SEQ ID NO: | DNA Identifier | Organism | binding domain |
| SEQ ID NOS: 1-2 | SMF69967.1 | Azospirillum oryzae A2P | 89 |
| SEQ ID NOS: 3-4 | WP_042442472.1 | Azospirillum lipoferum B510 | 89 |
| SEQ ID NOS: 5-6 | AIL32582.1 | Basilea psittacipulmonis | 97 |
| SEQ ID NOS: 7-8 | ADG66884.1 | Planctopirus limnophila | 96 |
| SEQ ID NOS: 9-10 | KHJ37904.1 | Pedobacter glucosidilyticus | 94 |
| SEQ ID NOS: 11-12 | WP_081748371.1 | Porphyromonas catoniae | 93 |
| SEQ ID NOS: 13-14 | WP_052080772.1 | Porphyromonas sp. COT-239 OH1446 | 94 |
| SEQ ID NOS: 15-16 | EHG19535.1 | Selenomonas infelix | 96 |
| SEQ ID NOS: 19-20 | WP_109445332 | Azospirillum sp. TSH64 | 89 |
| SEQ ID NOS: 21-22 | QCG87584.1 | Azospirillum sp. TSH100 | 89 |
| SEQ ID NOS: 27-28 | SEQ36152.1 | Butyrivibrio sp. TB | 93 |
| SEQ ID NOS: 29-30 | EKX99948.1 | Porphyromonas catoniae F0037 | 93 |
| SEQ ID NOS: 31-32 | SHI32494.1 | Butyrivibrio fibrisolvens DSM 3071 | 93 |

Furthermore, it was also found that the polypeptide sequences of SEQ ID NO: 6, SEQ ID NO: 10, SEQ ID NO: 12, SEQ ID NO: 14 and SEQ ID NO: 16, share the domain PENXXXXXXXTEK (SEQ ID NO: 37), wherein X can be any distinct amino acid, as shown in FIG. 1, wherein the domain is put in a box. All alignments were done with MAFFT v7.307, visualization was made with Jalview 2.10.

Furthermore, it was also found that, in addition to the conserved GDP-fucose binding domain [Y/W/L/H/F/M]X[T/S/C][E/Q/D/A][K/R] (SEQ ID NO: 33), the newly identified polypeptides all share the common conserved motif [K/D][L/K/M]XXX[F/Y] (SEQ ID NO: 34) wherein X can be any distinct amino acid, as well as the conserved amino acid region [FW]W that is important for lactose binding, as shown in FIG. 11, wherein the domains are put in a box.

In addition, we noticed that when this common feature is DM[A/S]VSF (SEQ ID NO: 36), additionally the conserved [N/H]XDPAXLD (SEQ ID NO: 35) motif, wherein X can be any distinct amino acid, is required in the N-terminal domain of the protein for the enzyme to have α-1,3-fucosyltransferase activity on lactose as the acceptor substrate, as shown in the alignment of FIG. 12, wherein the domains are put in a box. As exemplified in Example 14, the polypeptides with SEQ ID NO: 2, SEQ ID NO: 4, SEQ ID NO: 20 and SEQ ID NO: 22 contain both consensus motifs and appear to have alphα-1,3-fucosyltransferase activity on lactose as the acceptor substrate, while the polypeptides with SEQ ID NO: 24 and SEQ ID NO: 26 does not contain the N-terminal [NH]XDPAXLD motif, wherein X can be any distinct amino acid (SEQ ID NO: 35) and do show this activity.

Furthermore, it was also found that the polypeptide sequences of SEQ ID NO: 6, SEQ ID NO: 12, SEQ ID NO: 14, SEQ ID NO: 16, SEQ ID NO: 28, SEQ ID NO: 30 and SEQ ID NO: 32, share the domains K[IV]F[FL]XGEN (SEQ ID NO: 41) and RFPLW (SEQ ID NO: 42), wherein x can be any distinct amino acid, as shown in the alignment of FIG. 13, wherein the domain is put in a box.

In a second embodiment, the disclosure also relates to an isolated and/or synthesized polynucleotide encoding a polypeptide with lactose binding alphα-1,3-fucosyltransferase activity as described above.

Within the scope of the disclosure, the polynucleotide can be an allelic variant of a polynucleotide encoding any one of the amino acid sequences shown in SEQ ID NOS: 2, 6, 8, 10, 12, 14, 16, 20, 22, 28, 30, 32.

Accordingly, the disclosure also relates to an isolated and/or synthesized polynucleotide that encodes a polypeptide with α-1,3-fucosyltransferase activity and that comprises a sequence selected from the group consisting of: a) SEQ ID NOS: 1, 5, 7, 9, 11, 13, 15, 19, 21, 27, 29, 31 of the attached sequence listing; b) a nucleic acid sequence complementary to SEQ ID NOS: 1, 5, 7, 9, 11, 13, 15, 19, 21, 27, 29, 31; c) a nucleic acid sequence having 80% or more sequence identity to SEQ ID NOS: 1, 5, 7, 9, 11, 13, 15, 19, 21, 27, 29, 31.

Accordingly, the disclosure also relates to the 3-fucosyllactose obtained by the methods according to the disclosure, as well as to the use of a polynucleotide, the vector, host cells, microorganisms or the polypeptide as described above for the production of 3-fucosyllactose. The alphα-1,3-fucosyllactose may be used as food additive, prebiotic, symbiotic, for the supplementation of baby food, adult food or feed, or as either therapeutically or pharmaceutically active compound. With the novel methods, alphα-1,3-fucosyllactose can easily and effectively be provided, without the need for complicated, time and cost consuming synthetic processes.

Unless defined otherwise, all technical and scientific terms used herein generally have the same meaning as commonly understood by one of ordinary skill in the art to which this disclosure belongs. Generally, the nomenclature used herein and the laboratory procedures in cell culture, molecular genetics, organic chemistry and nucleic acid chemistry described above and below are those well-known and commonly employed in the art. Standard techniques are used for nucleic acid and peptide synthesis. Generally, enzymatic reactions and purification steps are performed according to the manufacturer's specifications.

Further advantages follow from the specific embodiments, the examples and the attached drawings.

It goes without saying that the abovementioned features and the features that are still to be explained below can be used not only in the respectively specified combinations, but also in other combinations or on their own, without departing from the scope of the disclosure.

The disclosure relates to the following specific embodiments:

1. A method for producing α-1,3-fucosyllactose, the method comprising the steps of:
a) providing a polypeptide with α-1,3-fucosyltransferase activity and with the ability to use lactose as acceptor substrate wherein the polypeptide comprises
   i) an amino acid sequence encoding a conserved GDP-fucose binding domain [Y/W/L/H/F/M]X[T/S/C][E/Q/D/A][K/R] (SEQ ID NO: 33);
   ii) an amino acid sequence encoding a conserved [K/D][L/K/M]XXX[F/Y] domain (SEQ ID NO: 34), and
   iii) wherein additionally the conserved motif [N/H]XDPAXLD (SEQ ID NO: 35) is present at the N-terminal region if the domain of ii) equals DM[A/S]VSF (SEQ ID NO: 36);
wherein X can be any distinct amino acid; and
wherein the C-terminus of the polypeptide has less than or equal to 100 amino acids starting from the first amino acid of the GDP-fucose binding domain;
b) contacting the polypeptide with α-1,3-fucosyltransferase activity of step a) with a mixture comprising GDP-fucose as donor substrate, and lactose as acceptor substrate, under conditions where the polypeptide catalyzes the transfer of a fucose residue from the donor substrate to the acceptor substrate,
thereby producing α-1,3-fucosyllactose
c) optionally, separating the α-1,3-fucosyllactose.

2. Method according to embodiment 1, wherein the polypeptide is provided in a cell free system.

3. Method according to embodiment 1, wherein the polypeptide is produced by a cell comprising a polynucleotide encoding the polypeptide.

4. Method according to any one of embodiments 1 or 3, wherein the GDP-fucose and/or lactose is provided by a cell producing the GDP-fucose and/or lactose.

5. A method according to any one of embodiments 1,3 or 4, the method comprising the steps of:
i) providing a cell genetically modified for the production of α-1,3-fucosyllactose, said cell comprising at least one nucleic acid sequence coding for an enzyme for α-1,3-fucosyllactose synthesis,
the cell comprising the expression of the polypeptide with α-1,3-fucosyltransferase activity and with the ability to use lactose as acceptor substrate
ii) cultivating the cell in a medium under conditions permissive for the production of α-1,3-fucosyllactose,
iii) preferably separating the α-1,3-fucosyllactose from the cultivation.

6. Method according to embodiment 3, the method comprising the steps of:
a) providing a host cell expressing the polypeptide with α-1,3-fucosyltransferase activity and with the ability to use lactose as acceptor substrate;
b) growing, under suitable nutrient conditions permissive for the production of the α-1,3-fucosyllactose, and permissive for the expression of the polypeptide with α-1,3-fucosyltransferase activity, the host cell;
c) providing simultaneously or subsequently to step b) a donor substrate GDP-fucose and the acceptor substrate lactose, in order for the α-1,3-fucosyltransferase polypeptide to catalyze the transfer of a fucose residue from GDP-fucose to lactose, thereby producing α-1,3-fucosyllactose;
d) optionally separating the α-1,3-fucosyllactose from the host cell or the medium of its growth.

7. A method according to any one of embodiments 5 or 6, wherein the host cell is transformed or transfected to express an exogenous polypeptide with α-1,3-fucosyltransferase activity and with the ability to use lactose as an acceptor substrate.

8. Method according to any one of embodiments 3 to 7, wherein the GDP-fucose and/or lactose is provided by an enzyme simultaneously expressed in the host cell or by the metabolism of the host cell.

9. The method of any one of embodiments 1 to 8, further comprising purification of α-1,3-fucosyllactose.

10. Method according to any one of the preceding embodiments, wherein the polypeptide is selected from the group consisting of:
i) any one of SEQ ID NOS: 6, 2, 4, 8, 10, 12, 14, 16, 20, 22, 28, 30 or 32 of the attached sequence listing;
ii) an amino acid sequence having 87% or more sequence identity to the full length amino acid sequence of SEQ ID NOS: 2, 20 or 22;
iii) an amino acid sequence having 80% or more sequence identity to the full length amino acid sequence of any one of SEQ ID NOS: 6, 8, 10, 12, 14, 16, 28, 30 or 32;
iv) a fragment of an amino acid sequence shown in SEQ ID NOS: 2, 20 or 22, wherein the fragment comprises at least 45 contiguous amino acids thereof;
v) a fragment of an amino acid sequence shown in any one of SEQ ID NOS: 6, 8, 10, 12, 14, 16, 28, 30 or 32, wherein the fragment comprises at least 10 contiguous amino acids thereof and has lactose binding alphα-1,3-fucosyltransferase activity;
optionally, the polypeptide is further modified by an N-terminal and/or C-terminal amino acid stretch.

11. Method for the production of 3-fucosyllactose according to any one of the preceding embodiments, the method further comprising at least one of the following steps:
i) adding to the culture medium a lactose feed comprising at least 50, more preferably at least 75, more preferably at least 100, more preferably at least 120, more preferably at least 150 gram of lactose per initial reactor volume, preferably in a continuous manner, and preferably so that the final volume of the culture medium is not more than three-fold, preferably not more than two-fold, more preferably less than 2-fold of the volume of the culture medium before the addition of the lactose feed;
ii) adding a lactose feed in a continuous manner to the culture medium over the course of 1 day, 2 days, 3 days, 4 days, 5 days by means of a feeding solution;
iii) adding a lactose feed in a continuous manner to the culture medium over the course of 1 day, 2 days, 3 days, 4 days, 5 days by means of a feeding solution and wherein the concentration of the lactose feeding solution is 50 g/L, preferably 75 g/L, more preferably 100 g/L, more preferably 125 g/L, more preferably 150 g/L, more preferably 175 g/L, more preferably 200 g/L, more preferably 225 g/L, more preferably 250 g/L, more preferably 275 g/L, more preferably 300 g/L, more preferably 325 g/L, more preferably 350 g/L, more preferably 375 g/L, more preferably, 400 g/L, more preferably 450 g/L, more preferably 500 g/L, even more preferably, 550 g/L, most preferably 600 g/L; and wherein preferably the pH of the solution is set between 3 and 7 and wherein preferably the temperature of the feed solution is kept between 20° C. and 80° C.;
iv) the method resulting in a 3-fucosyllactose concentration of at least 50 g/L, preferably at least 75 g/L, more preferably at least 90 g/L, more preferably at least 100 g/L, more preferably at least 125 g/L, more preferably at least 150 g/L, more preferably at least 175 g/L, more preferably at least 200 g/L in the final volume of the culture medium.

12. Host cell genetically modified for the production of α-1,3-fucosyllactose, wherein the host cell comprises at least one nucleic acid sequence coding for an enzyme involved in α-1,3-fucosyllactose synthesis; the cell comprising the expression of a polypeptide with α-1,3-fucosyltransferase activity and with the ability to use lactose as acceptor substrate, wherein the polypeptide comprises:
i) an amino acid sequence encoding a conserved GDP-fucose binding domain [Y/W/L/H/F/M]X[T/S/C][E/Q/D/A][K/R] (SEQ ID NO: 33);
ii) an amino acid sequence encoding a conserved [K/D][L/K/M]XXX[F/Y] domain (SEQ ID NO: 34), and
iii) wherein additionally the conserved motif [N/H]XDPAXLD (SEQ ID NO: 35) is present at the N-terminal region if the domain of ii) equals DM[A/S]VSF (SEQ ID NO: 36);
wherein X can be any distinct amino acid; and
wherein the C-terminus of the polypeptide has less than or equal to 100 amino acids starting from the first amino acid of the GDP-fucose binding domain.

13. Cell according to embodiment 12, the host cell comprising i) a sequence comprising a polynucleotide encoding the polypeptide with lactose binding alphα-1,3-fucosyltransferase activity, wherein the sequence is a sequence foreign to the host cell and wherein the sequence is integrated in the genome of the host cell, or ii) containing a vector comprising a polynucleotide encoding the polypeptide, wherein the polynucleotide being operably linked to control sequences recognized by a host cell transformed with the vector.

14. Cell according to any one of embodiments 12 or 13, wherein the polypeptide comprises an amino acid sequence selected from the group consisting of:
i) any one of SEQ ID NOS: 2, 4, 6, 8, 10, 12, 14, 16, 20, 22, 28, 30 or 32 of the attached sequence listing;
ii) an amino acid sequence having 87% or more sequence identity to the full length amino acid sequence of SEQ ID NOS: 2, 20 or 22;
iii) an amino acid sequence having 80% or more sequence identity to the full length amino acid sequence of any one of SEQ ID NOS: 6, 8, 10, 12, 14, 16, 28, 30 or 32;
iv) a fragment of an amino acid sequence shown in SEQ ID NOS: 2, 20 or 22, wherein the fragment comprises at least 45 contiguous amino acids thereof;
v) a fragment of an amino acid sequence shown in any one of SEQ ID NOS: 6, 8, 10, 12, 14, 16, 28, 30 or 32, wherein the fragment comprises at least 10 contiguous amino acids thereof and has lactose binding alphα-1,3-fucosyltransferase activity;

Optionally, the polypeptide is further modified by an N-terminal and/or C-terminal amino acid stretch.

15. Method according to any one of the embodiments 3 to 11 or cell according to any one of embodiments 12, 13 or 14, wherein the cell is selected from the group consisting of microorganism, plant, or animal cells, preferably, the microorganism is a bacterium, fungus or a yeast, preferably, the plant is a rice, cotton, rapeseed, soy, maize or corn plant, preferably, the animal is an insect, fish, bird or non-human mammal; preferably the cell is an Escherichia coli cell.

16. Host cell according to any one of embodiments 12 to 15, wherein the host cell is a cell of a bacterium, preferably of an Escherichia coli strain, more preferably of an Escherichia coli strain that is a K12 strain, even more preferably the Escherichia coli K12 strain is Escherichia coli MG1655.

17. Host cell according to any one of embodiments 12 to 15, wherein the host cell is a yeast cell.

18. Host cell according to any one of embodiments 12 to 17, wherein the polynucleotide encoding the polypeptide with lactose binding alphα-1,2-fucosyltransferase activity is adapted to the codon usage of the respective host cell.

19. Method for the production of α-1,3-fucosyllactose, comprising the steps of:
a) providing a cell according to any one of embodiments 12 to 18,
b) cultivating the cell in a medium under conditions permissive for the production of α-1,3-fucosyltransferase,
c) preferably, separating the α-1,3-fucosyltransferase from the cultivation.

20. Use of a host cell according to any one of embodiments 12 to 18 for the production of α-1,3-fucosyllactose.

21. Use of a polypeptide as described in the method of any one of embodiment 1 or 11 for the production of α-1,3-fucosyllactose.

22. A microorganism heterologously expressing a lactose binding alphα-1,3-fucosyltransferase polypeptide wherein the polypeptide comprises:
i) an amino acid sequence encoding a conserved GDP-fucose binding domain [Y/W/L/H/F/M]X[T/S/C][E/Q/D/A][K/R] (SEQ ID NO: 33);
ii) an amino acid sequence encoding a conserved [K/D][L/K/M]XXX[F/Y] domain (SEQ ID NO: 34), and
iii) wherein additionally the conserved motif [N/H]XDPAXLD (SEQ ID NO: 35) is present at the N-terminal region if the domain of ii) equals DM[A/S]VSF (SEQ ID NO: 36);
wherein X can be any distinct amino acid; and
wherein the C-terminus of the polypeptide has less than or equal to 100 amino acids starting from the first amino acid of the GDP-fucose binding domain.

23. Microorganism according to embodiment 22, wherein the polypeptide comprises an amino acid sequence selected from the group consisting of:
i) any one of SEQ ID NOS: 2, 4, 6, 8, 10, 12, 14, 16, 20, 22, 28, 30 or 32 of the attached sequence listing;
ii) an amino acid sequence having 87% or more sequence identity to the full length amino acid sequence of SEQ ID NOS: 2, 20 or 22;
iii) an amino acid sequence having 80% or more sequence identity to the full length amino acid sequence of any one of SEQ ID NOS: 6, 8, 10, 12, 14, 16, 28, 30 or 32;
iv) a fragment of an amino acid sequence shown in SEQ ID NOS: 2, 20 or 22, wherein the fragment comprises at least 45 contiguous amino acids thereof;
v) a fragment of an amino acid sequence shown in any one of SEQ ID NOS: 6, 8, 10, 12, 14, 16, 28, 30 or 32, wherein the fragment comprises at least 10 contiguous amino acids thereof and has lactose binding alphα-1,3-fucosyltransferase activity;

Optionally, the polypeptide is further modified by an N-terminal and/or C-terminal amino acid stretch.

24. Use of a microorganism according to embodiment 22 or 23 for the production of alphα-1,3-fucosyllactose.

25. The method of any one of embodiments 1 to 11, 15, or 19, further comprising a step of separating the alphα-1,3-fucosyllactose from the host cell or the medium of its growth.

26. The method of any one of embodiments 1 to 11, 15, 19 or 25, wherein the separation comprises at least one of the following steps: clarification, ultrafiltration, nanofiltration, reverse osmosis, microfiltration, activated charcoal or carbon treatment, tangential flow high-performance filtration, tangential flow ultrafiltration, affinity chromatography, ion exchange chromatography, hydrophobic interaction chromatography and/or gel filtration, ligand exchange chromatography.

27. The method of any one of embodiments 1 to 11, 15, 19, 25 or 26, further comprising purification of alphα-1,3-fucosyllactose.

28. The method of embodiment 27, wherein the purification of the alphα-1,3-fucosyllactose comprises at least one of the following steps: use of activated charcoal or carbon, use of charcoal, nanofiltration, ultrafiltration or ion exchange, use of alcohols, use of aqueous alcohol mixtures, crystallization, evaporation, precipitation, drying, spray drying or lyophilization.

29. The method of any one of embodiments 1 to 11, 15, 19, 25 to 28, wherein the polypeptide is produced in a fungal, yeast, bacterial, insect, animal and plant expression system.

30. The method of embodiment 29, wherein the host cell is a cell of a bacterium, preferably of an Escherichia coli strain, more preferably of an Escherichia coli strain that is a K12 strain, even more preferably the Escherichia coli K12 strain is Escherichia coli MG1655.

31. The method of embodiment 29, wherein the host cell is a yeast cell.

32. The method of any one of embodiments 1 to 11, 15, 19, 25 to 31, wherein the lactose concentration in the culture medium ranges from 50 to 150 g/L.

33. The method of any one of embodiments 1 to 11, 15, 19, 25 to 32 wherein the final concentration of 3-fucosyllactose ranges between 70 g/L to 200 g/L.

34. A method of any one of embodiments 1 to 11, 15, 19, 25 to 33 wherein the production results in a lactose concentration to 3-fucosyllactose concentration ratio of less than 1:5 at the end of fermentation.

35. A method of any one of embodiments 1 to 11, 15, 19, 25 to 34 wherein the production results in a 3-fucosyllactose purity of 80% or more at the end of fermentation.

36. A method for the production of α-1,3-fucosyllactose, the method comprising the steps of:
a) providing a polypeptide with α-1,3-fucosyltransferase activity and with the ability to use lactose as acceptor substrate
b) contacting the polypeptide with α-1,3-fucosyltransferase activity of step a) with a mixture comprising GDP-fucose as donor substrate, and lactose as acceptor substrate, under conditions where the polypeptide catalyzes the transfer of a fucose residue from the donor substrate to the acceptor substrate,
thereby producing α-1,3-fucosyllactose,
c) wherein the catalysis results in a lactose concentration to 3-fucosyllactose concentration ratio of less than 1:5 at the end of fermentation
d) optionally, separating the α-1,3-fucosyllactose.

37. A method for the production of α-1,3-fucosyllactose, the method comprising the steps of:
a) providing a polypeptide with α-1,3-fucosyltransferase activity and with the ability to use lactose as acceptor substrate
b) contacting the polypeptide with α-1,3-fucosyltransferase activity of step a) with a mixture comprising GDP-fucose as donor substrate, and lactose as acceptor substrate, under conditions where the polypeptide catalyzes the transfer of a fucose residue from the donor substrate to the acceptor substrate,
thereby producing α-1,3-fucosyllactose,
c) wherein the catalysis results in a 3-fucosyllactose purity of 80% or more at the end of fermentation,
d) optionally, separating the α-1,3-fucosyllactose.

38. Method for the production of 3-fucosyllactose comprising at least one of the following steps:
i) Adding to the culture medium a lactose feed comprising at least 50, more preferably at least 75, more preferably at least 100, more preferably at least 120, more preferably at least 150 gram of lactose per initial reactor volume, preferably in a continuous manner, and preferably so that the final volume of the culture medium is not more than three-fold, preferably not more than two-fold, more preferably less than 2-fold of the volume of the culture medium before the addition of the lactose feed;
ii) Adding a lactose feed in a continuous manner to the culture medium over the course of 1 day, 2 days, 3 days, 4 days, 5 days by means of a feeding solution;
iii) Adding a lactose feed in a continuous manner to the culture medium over the course of 1 day, 2 days, 3 days, 4 days, 5 days by means of a feeding solution and wherein the concentration of the lactose feeding solution is 50 g/L, preferably 75 g/L, more preferably 100 g/L, more preferably 125 g/L, more preferably 150 g/L, more preferably 175 g/L, more preferably 200 g/L, more preferably 225 g/L, more preferably 250 g/L, more preferably 275 g/L, more preferably 300 g/L, more preferably 325 g/L, more preferably 350 g/L, more preferably 375 g/L, more preferably, 400 g/L, more preferably 450 g/L, more preferably 500 g/L, even more preferably, 550 g/L, most preferably 600 g/L; and wherein preferably the pH of the solution is set between 3 and 7 and wherein preferably the temperature of the feed solution is kept between 20° C. and 80° C.;
the method resulting in a 3-fucosyllactose concentration of at least 50 g/L, preferably at least 75 g/L, more preferably at least 90 g/L, more preferably at least 100 g/L, more preferably at least 125 g/L, more preferably at least 150 g/L, more preferably at least 175 g/L, more preferably at least 200 g/L in the final volume of the culture medium and preferably a lactose concentration to 3FL concentration ratio lower than 1:5, more preferably 1:10, even more preferably 1:20, most preferably 1:40 in the final volume of the culture.

39. Method for the production of 3-fucosyllactose comprising at least one of the following steps:
i) Adding to the culture medium a lactose feed comprising at least 50, more preferably at least 75, more preferably at least 100, more preferably at least 120, more preferably at least 150 gram of lactose per initial reactor volume, preferably in a continuous manner, and preferably so that the final volume of the culture medium is not more than three-fold, preferably not more than two-fold, more preferably less than 2-fold of the volume of the culture medium before the addition of the lactose feed;
ii) Adding a lactose feed in a continuous manner to the culture medium over the course of 1 day, 2 days, 3 days, 4 days, 5 days by means of a feeding solution;
iii) Adding a lactose feed in a continuous manner to the culture medium over the course of 1 day, 2 days, 3 days, 4 days, 5 days by means of a feeding solution and wherein the concentration of the lactose feeding solution is 50 g/L, preferably 75 g/L, more preferably 100 g/L, more preferably 125 g/L, more preferably 150 g/L, more preferably 175 g/L, more preferably 200 g/L, more preferably 225 g/L, more preferably 250 g/L, more preferably 275 g/L, more preferably 300 g/L, more preferably 325 g/L, more preferably 350 g/L, more preferably 375 g/L, more preferably, 400 g/L, more preferably 450 g/L, more preferably 500 g/L, even more preferably, 550 g/L, most preferably 600 g/L; and wherein preferably the pH of the solution is set between 3 and 7 and wherein preferably the temperature of the feed solution is kept between 20° C. and 80° C.;
the method resulting in a 3-fucosyllactose concentration of at least 50 g/L, preferably at least 75 g/L, more preferably at least 90 g/L, more preferably at least 100 g/L, more preferably at least 125 g/L, more preferably at least 150 g/L, more preferably at least 175 g/L, more preferably at least 200 g/L in the final volume of the culture medium and preferably with a 3FL purity of 80% or more in the final volume of the culture.

The following drawings and examples will serve as further illustration and clarification of the present disclosure and are not intended to be limiting.

### BRIEF DESCRIPTION OF THE DRAWINGS

FIG. 1 shows an alignment of the polypeptide sequences of SEQ ID NO: 6, SEQ ID NO: 10, SEQ ID NO: 12, SEQ ID NO: 14 and SEQ ID NO: 16.

FIG. 2 shows normalized production of 3-fucosyllactose in a growth experiment.

FIG. 3 shows normalized production of 3-fucosyllactose in a growth experiment with low to high amounts of lactose in the medium.

FIG. 4 shows normalized production of 3-fucosyllactose in a growth experiment with low amounts of lactose in the medium.

FIG. 5 shows the percentage of lactose that is converted to 3-FL of one of the identified lactose binding alphα-1,3-fucosyltransferases.

FIG. 6 shows the percentage of lactose that is converted to 3-FL of different of the identified lactose binding alphα-1,3-fucosyltransferases driven by different promoters.

FIG. 7 shows the normalized production of 3-fucosyllactose of a further experiment.

FIG. 8 shows the normalized production of 3-fucosyllactose of a subset of the identified lactose binding alphα-1,3-fucosyltransferases driven by different promoters.

FIG. 9 shows the normalized production of 3-fucosyllactose of strains expressing H. pylori fucT (SEQ ID NO: 18) from 2 different promoters.

FIG. 10 shows the normalized production of 3-fucosyllactose of strains expressing polypeptides with the DM[AS]VSF consensus motif

FIGS. 11A-11C show an alignment of the polypeptide sequences of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 20, SEQ ID NO: 22, SEQ ID NO: 28, SEQ ID NO: 30 and SEQ ID NO: 32. The consensus motifs [Y/W/L/H/F/M]X[T/S/C][E/Q/D/A][K/R], [K/D][L/K/M]XXX[F/Y] and [FW]W, wherein X can be any distinct amino acid, are marked with a box.

FIG. 12 shows an alignment of the polypeptide sequences of SEQ ID NO: 2, SEQ ID NO: 4, SEQ ID NO: 20, SEQ ID NO: 22, SEQ ID NO: 24 and SEQ ID NO: 26. The consensus motifs DM[A/S]VSF and [N/H]XDPAXLD, wherein X can be any distinct amino acid (and unrelated motifs) are marked with a box.

FIG. 13 shows an alignment of the polypeptide sequences of SEQ ID NO: 6, SEQ ID NO: 12, SEQ ID NO: 14, SEQ ID NO: 16, SEQ ID NO: 28, SEQ ID NO: 30 and SEQ ID NO: 32. The consensus motifs K[I/V]F[F/L]XGEN (SEQ ID NO: 41) and RFPLW (SEQ ID NO: 42), wherein X can be any distinct amino acid, are marked with a box.

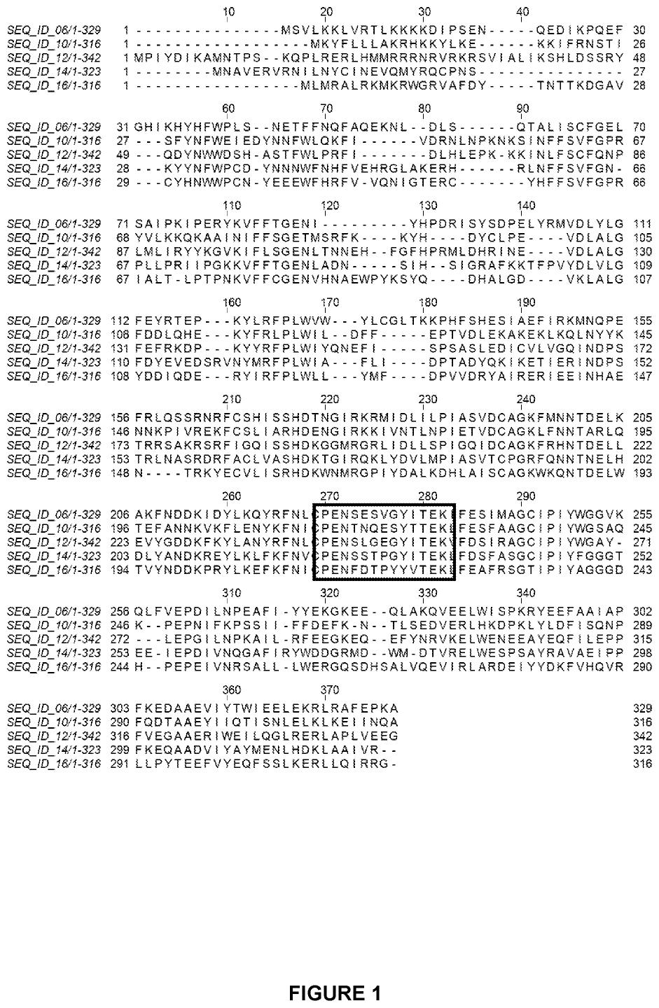

### DETAILED DESCRIPTION

### EXAMPLES

### Example 1

Materials and Methods Escherichia Coli Media

The Luria Broth (LB) medium consisted of 1% tryptone peptone (Difco, Erembodegem, Belgium), 0.5% yeast extract (Difco) and 0.5% sodium chloride (VWR. Leuven, Belgium). The medium for the shake flasks experiments contained 2.00 g/L NH₄Cl, 5.00 g/L (NH₄)2SO₄, 2.993 g/L KH₂PO₄, 7.315 g/L K₂HPO₄, 8.372 g/L MOPS, 0.5 g/L NaCl, 0.5 g/L MgSO₄.7H₂O, 14.26 g/L sucrose or another carbon source when specified in the examples, 1 ml/L vitamin solution, 100 μl/L molybdate solution, and 1 mL/L selenium solution. The medium was set to a pH of 7 with 1 M KOH. Vitamin solution consisted of 3.6 g/L FeCl₂.4H₂O, 5 g/L CaCl₂.2H₂O, 1.3 g/L MnCl₂.2H₂O, 0.38 g/L CuCl₂.2H₂O, 0.5 g/L CoCl₂.6H₂O, 0.94 g/L ZnCl₂, 0.0311 g/L H₃BO₄, 0.4 g/L Na₂EDTA.2H₂O and 1.01 g/L thiamine.HCl. The molybdate solution contained 0.967 g/L NaMoO₄.2H₂O. The selenium solution contained 42 g/L SeO₂.

The minimal medium for fermentations contained 6.75 g/L NH₄Cl, 1.25 g/L (NH₄)₂SO₄, 2.93 g/L KH₂PO₄ and 7.31 g/L KH₂PO₄, 0.5 g/L NaCl, 0.5 g/L MgSO₄.7H₂O, 14.26 g/L sucrose, 1 mL/L vitamin solution, 100 μL/L molybdate solution, and 1 mL/L selenium solution with the same composition as described above.

Complex medium was sterilized by autoclaving (121° C., 21 minutes) and minimal medium by filtration (0.22 μm Sartorius). When necessary, the medium was made selective by adding an antibiotic (e.g., chloramphenicol (20 mg/L), carbenicillin (100 mg/L), spectinomycin (40 mg/L) and/or kanamycin (50 mg/L)).

### Plasmids

pKD46 (Red helper plasmid, Ampicillin resistance), pKD3 (contains an FRT-flanked chloramphenicol resistance (cat) gene), pKD4 (contains an FRT-flanked kanamycin resistance (kan) gene), and pCP20 (expresses FLP recombinase activity) plasmids were obtained from Prof. R. Cunin (Vrije Universiteit Brussel, Belgium in 2007).

Plasmids for alphα-1,3-fucosyltransferase expression were constructed in a pMB1 ori vector using Golden Gate assembly. The genes were expressed using promoters apFAB305 (“PROM0012”), apFAB146 (“PROM0032”) (both as described by Mutalik et al. (Nat. Methods 2013, No. 10, 354-360)), and p14 (“PROM0016” in combination with “UTR0019”) (as described by De Mey et al. (BMC Biotechnology 2007)) and UTRs Gene10-LeuAB-BCD2 (“UTR0002”) (as described by Mutalik et al. (Nat. Methods 2013, No. 10, 354-360)).

Plasmids were maintained in the host E. coli DH5alpha (F⁻, phi80dlacZdeltaM15, delta(lacZYA-argF) U169, deoR, recA1, endA1, hsdR17(rk⁻, mk⁺), phoA, supE44, lambda, thi-1, gyrA96, relA1) bought from Invitrogen.

### Strains and Mutations

Escherichia coli K12 MG1655 [lambda⁻, F⁻, rph-1] was obtained from the Coli Genetic Stock Center (US), CGSC Strain#: 7740, in March 2007. Gene disruptions as well as gene introductions were performed using the technique published by Datsenko and Wanner (PNAS 97 (2000), 6640-6645). This technique is based on antibiotic selection after homologous recombination performed by lambda Red recombinase. Subsequent catalysis of a flippase recombinase ensures removal of the antibiotic selection cassette in the final production strain.

Transformants carrying a Red helper plasmid pKD46 were grown in 10 ml LB media with ampicillin, (100 mg/L) and L-arabinose (10 mM) at 30° C. to an OD600 nm of 0.6. The cells were made electrocompetent by washing them with 50 ml of ice-cold water, a first time, and with 1 ml ice cold water, a second time. Then, the cells were resuspended in 50 μl of ice-cold water. Electroporation was done with 50 μl of cells and 10-100 ng of linear double-stranded-DNA product by using a Gene Pulser™ (BioRad) (600 Ω, 25 μED, and 250 volts).

After electroporation, cells were added to 1 ml LB media incubated 1 hour at 37° C., and finally spread onto LB-agar containing 25 mg/L of chloramphenicol or 50 mg/L of kanamycin to select antibiotic resistant transformants. The selected mutants were verified by PCR with primers upstream and downstream of the modified region and were grown in LB-agar at 42° C. for the loss of the helper plasmid. The mutants were tested for ampicillin sensitivity.

The linear ds-DNA amplicons were obtained by PCR using pKD3, pKD4 and their derivates as template. The primers used had a part of the sequence complementary to the template and another part complementary to the side on the chromosomal DNA where the recombination must take place. For the genomic knock-out, the region of homology was designed 50-nt upstream and 50-nt downstream of the start and stop codon of the gene of interest. For the genomic knock-in, the transcriptional starting point (+1) had to be respected. PCR products were PCR-purified, digested with Dpn1, repurified from an agarose gel, and suspended in elution buffer (5 mM Tris, pH 8.0).

The selected mutants (chloramphenicol or kanamycin resistant) were transformed with pCP20 plasmid, which is an ampicillin and chloramphenicol-resistant plasmid that shows temperature-sensitive replication and thermal induction of FLP synthesis. The ampicillin-resistant transformants were selected at 30° C., after which a few were colony purified in LB at 42° C. and then tested for loss of all antibiotic resistance and of the FLP helper plasmid. The gene knock outs and knock ins are checked with control primers (Fw/Rv-gene-out).

A mutant strain derived from E. coli K12 MG1655 was created by knocking out the genes lacZ, lacY lacA, glgC, agp, pfkA, pflth, pgi, arcA, iclR, wcaf, pgi, ion and thyA. Additionally, the E. coli lacY gene, a fructose kinase gene (frk) originating from Zymomonas mobilis and a sucrose phosphorylase (SP) originating from Bifidobacterium adolescentis were knocked in into the genome and expressed constitutively. The constitutive promoters originate from the promoter library described by De Mey et al. (BMC Biotechnology, 2007). These genetic modifications are also described in WO2016075243 and WO2012007481.

All constructed plasmids with the hypothetical alphα-1,3-fucosyltransferase genes were evaluated in this mutant strain derived from E. coli K12 MG1655. All strains are stored in cryovials at −80° C. (overnight LB culture mixed in a 1:1 ratio with 70% glycerol). A list of all successful lactose binding alphα-1,3-fucosyltransferases (SEQ ID NOS: 1 to 16, 19 to 22 and 27 to 32) together with a prior art alphα-1,3-fucosyltransferase (SEQ ID NOS: 17-18) and two non-functional alphα-1,3-fucosyltransferases (SEQ ID NOS: 23 to 26) is provided in Table 3.

TABLE 3

| SEQ ID | Organism | Country origin |
|---|---|---|
| SEQ ID NOS: 1-2 | Azospirillum oryzae A2P | Japan |
| SEQ ID NOS: 3-4 | Azospirillum lipoferum B510 | Japan |
| SEQ ID NOS: 5-6 | Basilea psittacipulmonis | Switzerland |
| SEQ ID NOS: 7-8 | Planctopirus limnophila (strain ATCC 43296/DSM 3776/ | Germany |
| | IFAM 1008/290) (Planctomyces limnophilus) | |
| SEQ ID NOS: 9-10 | Pedobacter glucosidilyticus | Germany |
| SEQ ID NOS: 11-12 | Porphyromonas catoniae (WGS, in genbank: | United States |
| | JDFF01000001 till JDFF010000025) | |
| SEQ ID NOS: 13-14 | Porphyromonas sp. COT-239 OH1446 (contig_18; | United Kingdom |
| | NZ_JRA001000018.1) | |
| SEQ ID NOS: 15-16 | Selenomonas infelix ATCC 43532 | Unknown |
| SEQ ID NOS: 17-18 | Helicobacter pylori | Australia |
| SEQ ID NOS: 19-20 | Azospirillum sp. TSH64 | Japan |
| SEQ ID NOS: 21-22 | Azospirillum sp. TSH100 | Japan |
| SEQ ID NOS: 23-24 | Azospirillum brasilense | Unknown |
| SEQ ID NOS: 25-26 | Azospirillum sp. B510 | Japan |
| SEQ ID NOS: 27-28 | Butyrivibrio sp. TB | Unknown |
| SEQ ID NOS: 29-30 | Porphyromonas catoniae F0037 | Unknown |
| SEQ ID NOS: 31-32 | Butyrivibrio fibrisolvens DSM 3071 | Unknown |

### Heterologous and Homologous Expression

All potential alphα-1,3-fucosyltransferase genes that needed to be expressed, be it for a plasmid or for the genomic insertion, were synthetically synthetized at Twist Biosciences (San Francisco, USA). Expression could be further facilitated by optimizing the codon usage to the codon usage of the expression host. Genes were optimized using the tools of the supplier.

### Cultivation Conditions

A preculture of 96-well microtiter plate experiments was started from a cryovial, in 150 μL LB and was incubated overnight at 37° C. on an orbital shaker at 800 rpm. This culture was used as inoculum for a 96-well square microtiter plate, with 400 μL MMsf medium by diluting 400×. These final 96-well culture plates were then incubated at 37° C. on an orbital shaker at 800 rpm for 72 hours, or shorter, or longer. At the end of the cultivation experiment samples were taken from each well to measure sugar concentrations in the broth supernatant (extracellular sugar concentrations, after spinning down the cells), or by boiling the culture broth for 15 minutes at 90° C. before spinning down the cells (=whole broth measurements, average of intra- and extracellular sugar concentrations).

A preculture for the bioreactor was started from an entire 1 mL cryovial of a certain strain, inoculated in 250 mL or 500 mL of MMsf medium in a 1 L or 2.5 L shake flask and incubated for 24 hours at 37° C. on an orbital shaker at 200 rpm. A 5 L bioreactor was then inoculated (250 mL inoculum in 2 L batch medium); the process was controlled by MFCS control software (Sartorius Stedim Biotech, Melsungen, Germany). Culturing conditions were set to 37° C., and maximal stirring; pressure gas flow rates were dependent on the strain and bioreactor. The pH was controlled at 6.8 using 0.5 M H2S04 and 20% NH4OH. The exhaust gas was cooled. 10% solution of silicone antifoaming agent was added when foaming raised during the fermentation.

### Optical Density

Cell density of the cultures was frequently monitored by measuring optical density at 600 nm (Implen Nanophotometer NP80, Westburg, Belgium or with a Spark 10 M microplate reader, Tecan, Switzerland).

### Liquid Chromatography

Standards for 3-fucosyllactose were synthetized in house. Other standards such as but not limited to lactose, sucrose, glucose, fructose were purchased from Sigma.

Carbohydrates were analyzed via a HPLC-RI (Waters, USA) method, whereby RI (Refractive Index) detects the change in the refraction index of a mobile phase when containing a sample. The sugars were separated in an isocratic flow using an X-Bridge column (Waters X-bridge HPLC column, USA) and a mobile phase containing 75 ml acetonitrile and 25 ml Ultrapure water and 0.15 ml triethylamine. The column size was 4.6×150 mm with 3.5 μm particle size. The temperature of the column was set at 35° C. and the pump flow rate was 1 mL/minute.

### Example 2

### Evaluation of Different Lactose Binding alphα-1,3-Fucosyltransferase Enzymes Incorporated in Escherichia Coli

An experiment was set up to evaluate a number of genes coding for potential alphα-1,3-fucosyltransferase enzymes that are able to produce 3-fucosyllactose (3-FL) from GDP-fucose and lactose. A growth experiment was performed according to the cultivation conditions provided in Example 1.

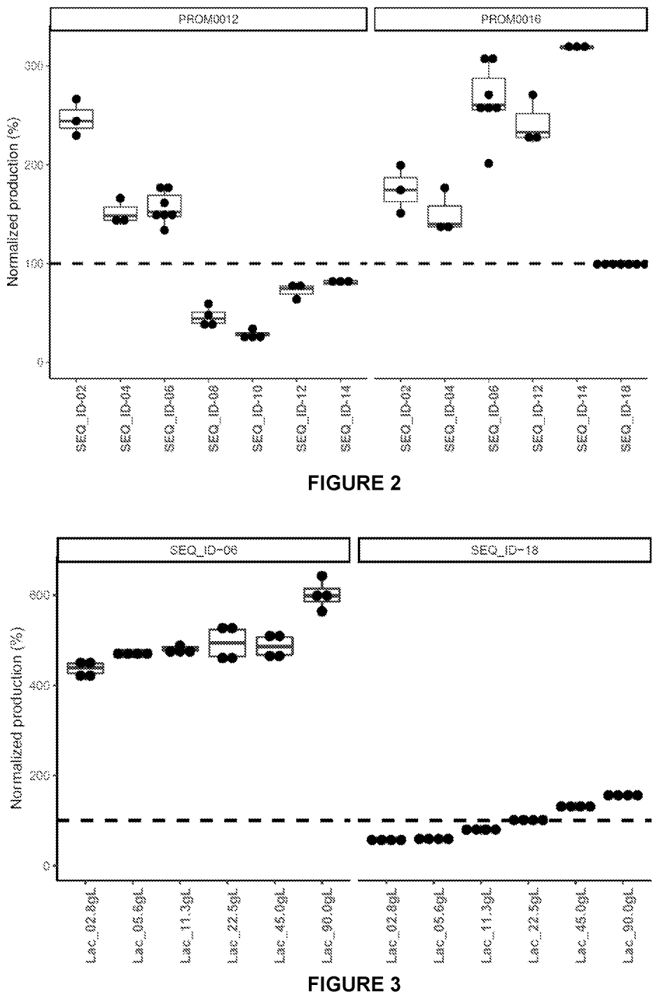

FIG. 2 shows the normalized production of 3-fucosyllactose obtained in a growth experiment of the strains successfully expressing various lactose binding alphα-1,3-fucosyltransferases using two different promoters (PROM0012 and PROM0016) with 20 g/L lactose in the production medium. Each datapoint corresponds to data from one well. The dashed horizontal line indicates the setpoint to which all datapoints were normalized.

The experiment identified the following polypeptides with lactose binding 3-fucosyltransferase activity: SEQ ID NO: 2, SEQ ID NO: 4, SEQ ID NO: 6, SEQ ID NO: 8, SEQ ID NO: 10, SEQ ID NO: 12 and SEQ ID NO: 14 having similar to better lactose binding α-1,3-fucosyltransferase activity compared to a strain containing SEQ ID NO: 18 with previously confirmed lactose binding α-1,3-fucosyltransferase activity. The polypeptide of SEQ ID NO: 4 has 90.8% global sequence identity to SEQ ID NO: 2, herewith showing that also sequences that have 87% or more sequence identity to SEQ ID NO: 2 have lactose binding α-1,3-fucosyltransferase activity.

### Example 3

### Evaluation of a Lactose Binding alphα-1,3-Fucosyltransferase Enzyme Incorporated in Escherichia Coli for its Ability to Produce 3-FL with Low to High Lactose Concentrations in Minimal Media

A gene coding for SEQ ID NO: 6 (and combined with PROM0016) is evaluated on its ability to produce 3-FL in minimal media with various concentrations of lactose. A growth experiment was performed according to the cultivation conditions provided in Example 1. Strains with SEQ ID NO: 6 and SEQ ID NO: 18 (driven by PROM0016) were grown in multiple wells of a 96-well plate as described above. SEQ ID NO: 18 has previously confirmed alphα-1,3-fucosyltransferase activity on lactose.

FIG. 3 shows the normalized production of 3-fucosyllactose with six different concentrations of lactose as a precursor for 3-FL (90 g/L and a 1:2 dilution series thereof, until 2.8 g/L, as indicated in the figure). Each datapoint corresponds to data from one well. The dashed horizontal line indicates the setpoint to which all datapoints were normalized.

The experiment identified the polypeptide of SEQ ID NO: 6 to have better lactose binding α-1,3-fucosyltransferase activity at all lactose concentrations compared to a strain expressing SEQ ID NO: 18, a polypeptide with previously confirmed lactose binding alphα-1.3-fucosyltransferase activity.

### Example 4

### Evaluation of Various Lactose Binding alphα-1,3-Fucosyltransferase Enzymes Incorporated in Escherichia Coli for Their Ability to Produce 3-FL at Low Concentrations of Lactose in Minimal Media

Several of the above identified strains with genes coding for SEQ ID NO: 2, SEQ ID NO: 4, SEQ ID NO: 6, SEQ ID NO: 12 and SEQ ID NO: 14 were evaluated on their ability to produce 3-fucosyllactose from GDP-fucose and lactose in a growth experiment at low concentrations of lactose. A growth experiment was performed according to the cultivation conditions provided in Example 1.

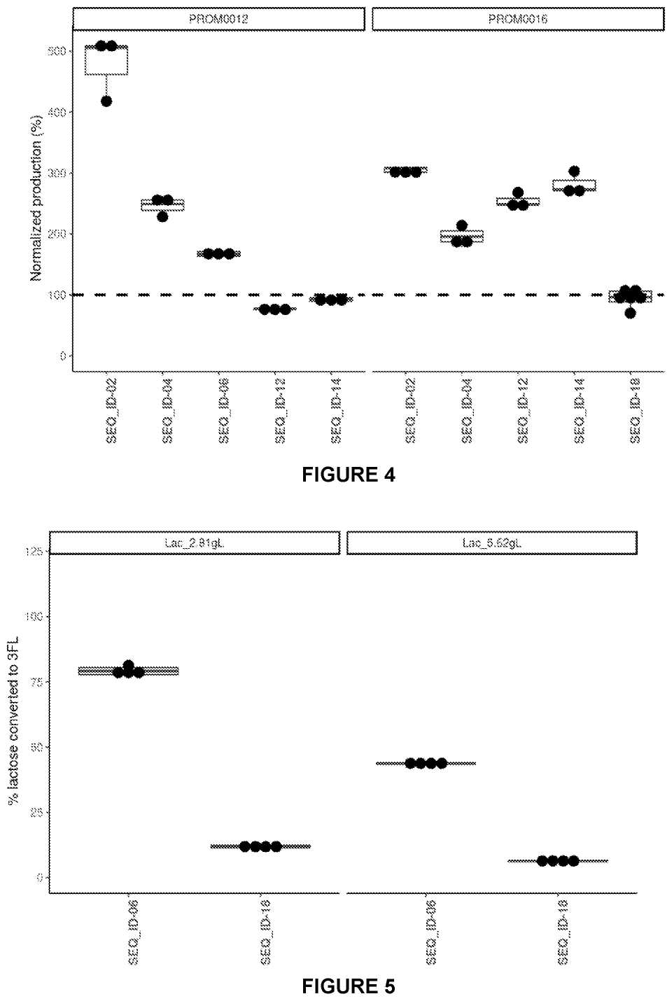

FIG. 4 shows the normalized production of 3-fucosyllactose with strains expressing various alphα-1,3-fucosyltransferases (using two different promoters PROM0012 and PROM0016) and grown in a medium with low amounts of lactose (2.8 g/L lactose). Each datapoint corresponds to data from one well. The dashed horizontal line indicates the setpoint to which all datapoints were normalized.

The experiment identified the following polypeptides with SEQ ID NO: 2, SEQ ID NO: 4, SEQ ID NO: 6, SEQ ID NO: 12 and SEQ ID NO: 14 to having similar to better lactose binding alphα-1,3-fucosyltransferase activity when provided with low concentrations of lactose compared to a strain containing SEQ ID NO: 18 with previously confirmed lactose binding alpha-1,3-fucosyltransferase activity.

### Example 5

### Evaluation of Enzyme Activity of the Polypeptide of SEQ ID NO: 6 Incorporated in Escherichia Coli on Two Low Concentrations of Lactose

A gene coding for SEQ ID NO: 6 (and combined with PROM0016) was evaluated for its ability to convert lactose into 3-fucosyllactose in a strain producing GDP-fucose in a growth experiment providing 2.8 g/L or 5.62 g/L of lactose and sucrose at 30 g/L. A growth experiment was performed according to the cultivation conditions provided in Example 1.

FIG. 5 shows the percentage of lactose that is converted to 3-FL, calculated by dividing the measured amount of 3-FL by the amount that could theoretically be obtained based on the input concentration of lactose. Theoretically, if all lactose is converted, a value of 100% is obtained. Each datapoint corresponds to data from one well.

The strain expressing polypeptide as shown in SEQ ID NO: 6 is compared to a strain expressing the polypeptide as shown in SEQ ID NO: 18 (driven by PROM0016), which is previously confirmed to have alphα-1,3-fucosyltransferase activity on lactose. At both concentrations of lactose the strain expressing the polypeptide as shown in SEQ ID NO: 6 is able to convert much more lactose to 3-FL than the strain expressing the polypeptide as shown in SEQ ID NO: 18 for a given amount of carbon source (30 g/L of sucrose).

### Example 6

### Evaluation of Enzyme Activity of Various Lactose Binding alphα-1,3-Fucosyltransferase Enzymes Incorporated in Escherichia Coli at Limited Concentrations of Lactose and Sucrose

Genes coding for the above identified polypeptides SEQ ID NO: 2, SEQ ID NO: 6, SEQ ID NO: 12 and SEQ ID NO: 14 were evaluated on their ability to convert lactose into 3-fucosyllactose in a strain producing GDP-fucose in a growth experiment at low concentrations of lactose (2 g/L) and sucrose (7.5 g/L). A growth experiment was performed according to the cultivation conditions provided in Example 1.

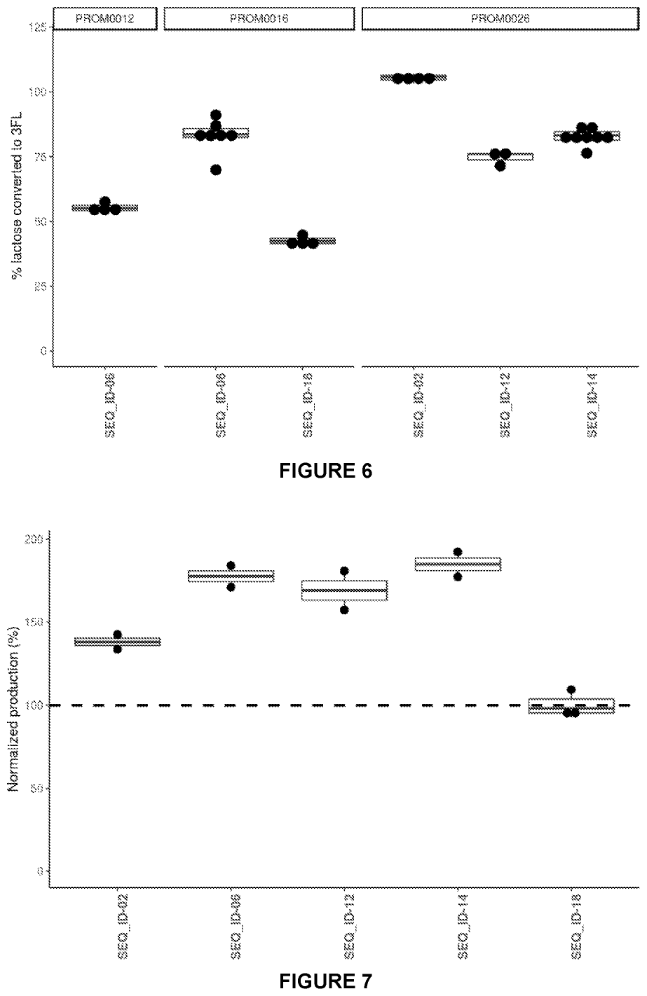

FIG. 6 shows the percentage of lactose that is converted to 3-FL, calculated by dividing the measured amount of 3-FL by the amount that could theoretically be obtained based on the input concentration of lactose. Each datapoint corresponds to data from one well.

The strains expressing the polypeptides with SEQ ID NO: 2, SEQ ID NO: 6, SEQ ID NO: 12 or SEQ ID NO: 14 are able to convert more lactose to 3-FL than the strain expressing the polypeptide with SEQ ID NO: 18 for a given amount of carbon source (7.5 g/L sucrose).

### Example 7

### Evaluation of Escherichia Coli Strains Expressing Various Lactose Binding alphα-1,3-Fucosyltransferase Enzymes in a Batch Fermentation

Batch fermentations at bioreactor scale were performed to evaluate strains, derived from the mutant E. coli K12 MG1655 strain background as described in Example 1, expressing various alphα-1,3-fucosyltransferase enzymes with SEQ ID NO: 2, SEQ ID NO: 6, SEQ ID NO: 12 and SEQ ID NO: 14. The bioreactor runs were performed as described in Example 1. In these examples, sucrose was used as a carbon source. Lactose was added in the batch medium at 90 g/L as a precursor for 3-FL formation.

FIG. 7 shows the normalized production of 3-fucosyllactose obtained in batch fermentations with strains successfully expressing various lactose binding alphα-1,3-fucosyltransferases with lactose in the production medium as a precursor. Each datapoint corresponds to data from one fermentation run. The dashed horizontal line indicates the setpoint to which all datapoints were normalized.

The experiment shows that mutant E. coli strains expressing the lactose binding alphα-1,3-fucosyltransferase genes with SEQ ID NO: 2, SEQ ID NO: 6, SEQ ID NO: 12 or SEQ ID NO: 14 produce higher amounts of 3-FL compared to the strain expressing the polypeptide with SEQ ID NO: 18.

### Example 8

### Evaluation of Different Lactose Binding alphα-1,3-Fucosyltransferase Enzymes Incorporated in Escherichia Coli

A further experiment was set up with strains expressing the enzymes with SEQ ID NO: 2, SEQ ID NO: 6, SEQ ID NO: 12, SEQ ID NO: 14 and SEQ ID NO: 16 and evaluated whether these are able to produce 3-fucosyllactose from lactose in a strain producing GDP-fucose. A growth experiment was performed according to the cultivation conditions provided in Example 1.

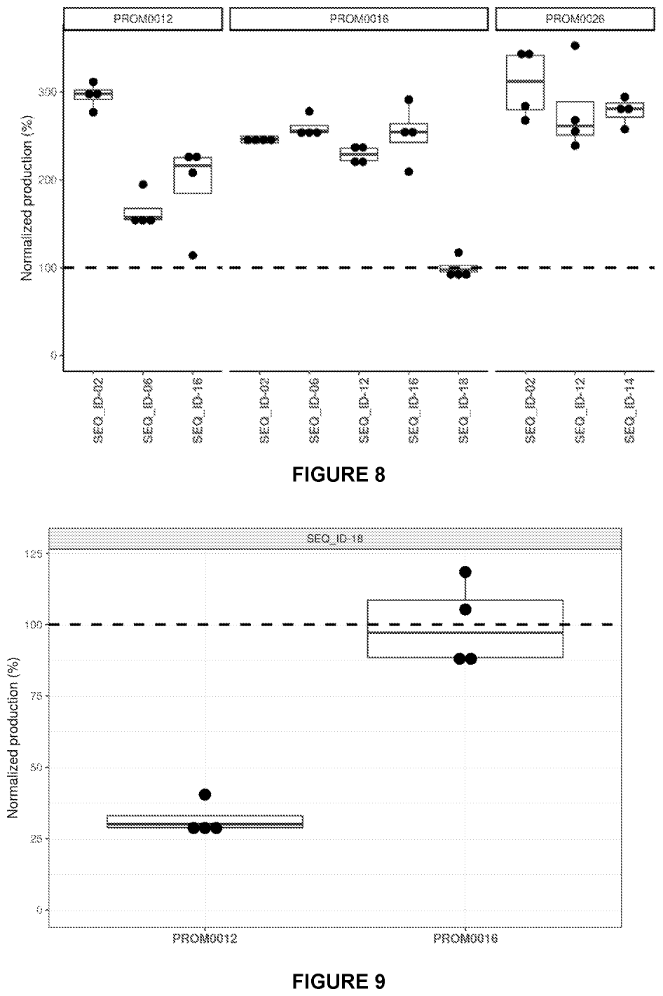

FIG. 8 shows normalized production of 3-fucosyllactose with strains successfully expressing various lactose binding alphα-1,3-fucosyltransferases (using three different promoters PROM0012, PROM0016 and PROM0026) with 20 g/L lactose in the production medium. Each datapoint corresponds to data from one well. The dashed horizontal line indicates the setpoint to which all datapoints were normalized.

The experiment confirmed the results from Example 2 for the strains expressing polypeptides with SEQ ID NO: 12, SEQ ID NO: 6, SEQ ID NO: 12 and SEQ ID NO: 14, and identified the polypeptide with SEQ ID NO: 16 to also have better lactose binding alphα-1,3-fucosyltransferase activity compared to a strain containing SEQ ID NO: 18 with previously confirmed lactose binding alphα-1,3-fucosyltransferase activity.

### Example 9

### Material and Methods Saccharomyces Cerevisiae Media

Strains are grown on Synthetic Defined yeast medium with Complete Supplement Mixture (SD CSM) or CSM drop-out (SD CSM-Ura) containing 6.7 g/L Yeast Nitrogen Base without amino acids (YNB w/o AA, Difco), 20 g/L agar (Difco) (solid cultures), 22 g/L glucose monohydrate or 20 g/L lactose and 0.79 g/L CSM or 0.77 g/L CSM-Ura (MP Biomedicals).

### Strains

Saccharomyces cerevisiae BY4742 created by Bachmann et al. (Yeast (1998) 14:115-32) was used available in the Euroscarf culture collection. All mutant strains were created by homologous recombination or plasmid transformation using the method of Gietz (Yeast 11:355-360, 1995). Kluyveromyces marxianus lactis is available at the LMG culture collection (Ghent, Belgium).

### Plasmids

Yeast expression plasmid p2a_2 μ_sia_GFA1 (Chan 2013 (Plasmid 70 (2013) 2-17)) was used for expression of foreign genes in Saccharomyces cerevisiae. This plasmid contains an ampicillin resistance gene and a bacterial origin of replication to allow for selection and maintenance in E. coli. The plasmid further contains the 2 μ yeast ori and the Ura3 selection marker for selection and maintenance in yeast. Next, this plasmid can be modified to p2_a2 μ_ff to contain a lactose permease (for example, LAC12 from Kluyveromyces lactis), a GDP-mannose 4,6-dehydratase (such as Gmd from E. coli) and a GDP-L-fucose synthase (such as fcl from E. coli).

Yeast expression plasmids p2a 2 μ_fl_3ft is based on p2a 2₁1. ft but modified in a way that also SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16 or SEQ ID NO: 18 are expressed. Preferably but not necessarily, the fucosyltransferase proteins are N-terminally fused to a SUMOstar tag (e.g., obtained from pYSUMOstar, Life Sensors, Malvern, Pa.) to enhance the solubility of the fucosyltransferase enzymes.

Plasmids were maintained in the host E. coli DH5alpha (F⁻, phi80diacZdeltaM15, delta(/acZYA-argF)U169, deoR, recA1, endA1, hsdR17(rk⁺, mk⁺), phoA, supE44, lambda⁻, thi-1, gyrA96, rel A2) bought from Invitrogen.

### Gene Expression Promoters

Genes are expressed using synthetic constitutive promoters, as described in by Blazeck (Biotechnology and Bioengineering, Vol. 109, No. 11, 2012).

### Heterologous and Homologous Expression

Genes that needed to be expressed, be it from a plasmid or from the genome were synthetically synthetized with one of the following companies: DNA2.0, Gen9 or IDT.

Expression could be further facilitated by optimizing the codon usage to the codon usage of the expression host. Genes were optimized using the tools of the supplier.

### Cultivations Conditions

In general, yeast strains were initially grown on SD CSM plates to obtain single colonies. These plates were grown for 2-3 days at 30° C.

Starting from a single colony, a preculture was grown over night in 5 mL at 30° C., shaking at 200 rpm. Subsequent 125 mL shake flask experiments were inoculated with 2% of this preculture, in 25 mL media. These shake flasks were incubated at 30° C. with an orbital shaking of 200 rpm. The use of an inducer is not required as all genes are constitutively expressed.

### Example 10

### Production of 3-Fucosyllactose in Saccharomyces Cerevisiae Using Various Lactose Binding alphα-1,3-Fucosyltransferase Enzymes

Another example provides use of a eukaryotic organism, in the form of Saccharomyces cerevisiae, for the disclosure. Using the strains, plasmids and methods as described in Example 9, strains are created that express SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16 or SEQ ID NO: 18.

On top of that, further modifications are made in order to produce 3-fucosyllactose. These modifications comprise the addition of a lactose permease, a GDP-mannose 4,6-dehydratase and a GDP-L-fucose synthase. The preferred lactose permease is the KlLAC12 gene from Kluyveromyces lactis (WO 2016/075243). The preferred GDP-mannose 4,6-dehydratase and the GDP-L-fucose synthase are respectively gmd and fcl from Escherichia coli.

These strains are capable of growing on glucose or glycerol as carbon source, converting the carbon source into GDP-L-fucose, taking up lactose, and producing 3-fucosyllactose using GDP-L-fucose and lactose as substrates for the enzymes represented by SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16 or SEQ ID NO: 18, with SEQ ID NO: 18 as reference.

Preculture of the strains are made in 5 mL of the synthetic defined medium SD-CSM containing 22 g/L glucose and grown at 30° C. as described in Example 9. These precultures are inoculated in 25 mL medium in a shake flask with 10 g/L sucrose as sole carbon source and grown at 30° C. Regular samples are taken and the production of 3-fucosyllactose is measured as described in Example 1.

### Example 11

### Enzymatic Production of 3-Fucosyllactose

Another example provides the use of an enzyme with SEQ ID NO: 2, SEQ ID NO: 4, SEQ ID NO: 6, SEQ ID NO: 8, SEQ ID NO: 10, SEQ ID NO: 12, SEQ ID NO: 14 or SEQ ID NO: 16 of the present disclosure. These enzymes are produced in a cell-free expression system such as but not limited to the PURExpress system (NEB), or in a host organism such as but not limited to Escherichia coli or Saccharomyces cerevisiae, after which the above-listed enzymes can be isolated and optionally further purified.

Each of the above enzyme extracts or purified enzymes are added to a reaction mixture together with GDP-fucose, lactose and a buffering component such as Tris-HCl or HEPES. The reaction mixture is then incubated at a certain temperature (for example, 37° C.) for a certain amount of time (for example, 24 hours), during which the lactose will be converted to 3-fucosyllactose by the enzyme using GDP-fucose. The 3-fucosyllactose is then separated from the reaction mixture by methods known in the art. Further purification of the 3-FL can be performed if preferred. At the end of the reaction or after separation and/or purification, the production of 3-fucosyllactose is measured as described in Example 1.

### Example 12

### 3-Fucosyllactose Production with Different Lactose Concentrations

A fermentation process as described in Examples 1 and 7, wherein the lactose concentration in the culture medium ranges from 50 to 150 g/L. The lactose is converted during the process into 3-fucosyllactose until minor amounts of lactose is left. The final ratio lactose to 3-fucosyllactose may be manipulated during this process by stopping the process earlier (higher lactose to 3-fucosyllactose ratio) or later (lower lactose to 3-fucosyllactose ratio) The lactose concentration may be increased in the vessel by feeding high concentrations of lactose solution with or without another carbon source to the bioreactor. The lactose feed contains lactose concentrations between 100 and 700 g/L and is kept at a temperature so that the lactose is kept soluble at a pH below or equal to 6 to avoid lactulose formation during the process, a standard method used in the dairy industry. The final concentrations of 3-fucosyllactose reached in such a production process ranges between 70 g/L when lower lactose concentrations are used and 200 g/L or higher when high lactose concentrations are used in the process as described above.

### Example 13

### Evaluation of the Helicobacter Pylori alphα-1,3-Fucosyltransferase fucT (SEQ ID NO: 18) Expressed from Various Promoters

The gene coding for the H. pylori alphα-1,3-fucosyltransferase fucT (SEQ ID NO: 18) was cloned in an expression vector under control of promoters PROM0012 or PROM0016, and the resulting plasmids were transformed to the E. coli mutant strain as described in Example 1. These strains were then evaluated in a growth experiment for their ability to produce 3-FL. Both strains were grown in multiple wells of a 96-well plate.

FIG. 9 shows the normalized production of 3-fucosyllactose produced by the strains. Each datapoint corresponds to data from one well. The dashed horizontal line indicates the setpoint to which all datapoints were normalized.

The experiment shows that the 3-FL production in a strain expressing H. pylori FucT using promoter PROM0012 drops to ±30% of the levels observed for a similar strain expressing the fucosyltransferase from promoter PROM0016.

By extrapolation of the data provided in Examples 2, 4 and 8, we can conclude that all strains containing any of the SEQ ID NOS: 2-16 show a significantly higher production compared to the control strain with α-1,3-fucosyltransferase fucT (SEQ ID NO: 18) when the fucosyltransferase is expressed from the same promoter (PROM0012 OR PROM0016), except for the strain with SEQ ID NO: 10, which shows a similar production as the control strain.

### Example 14

### Evaluation of Strains Expressing Polypeptides with the DMIASIVSF Consensus Motif for the Production of 3-Fucosyllactose

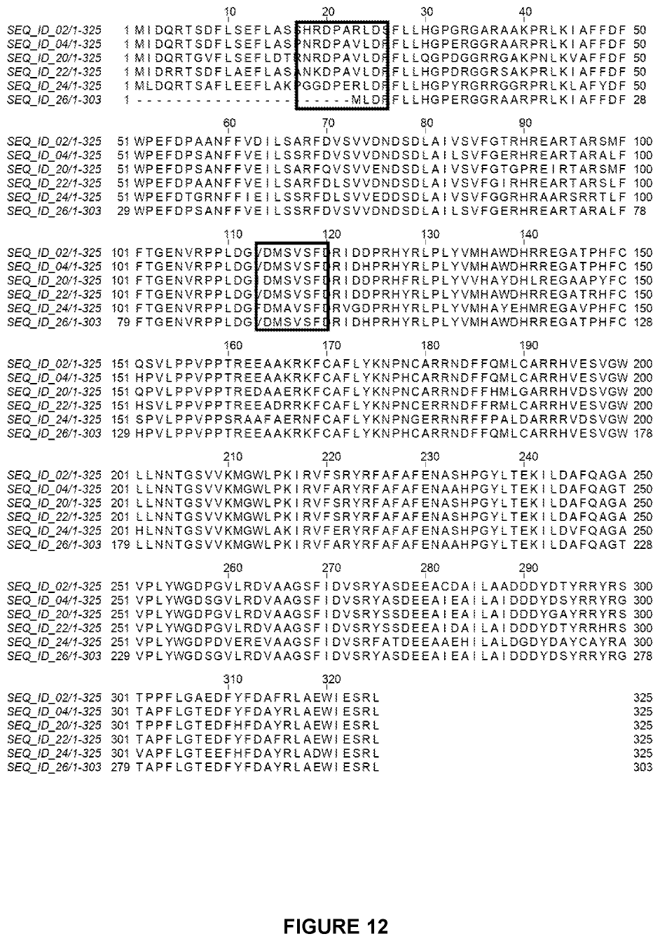

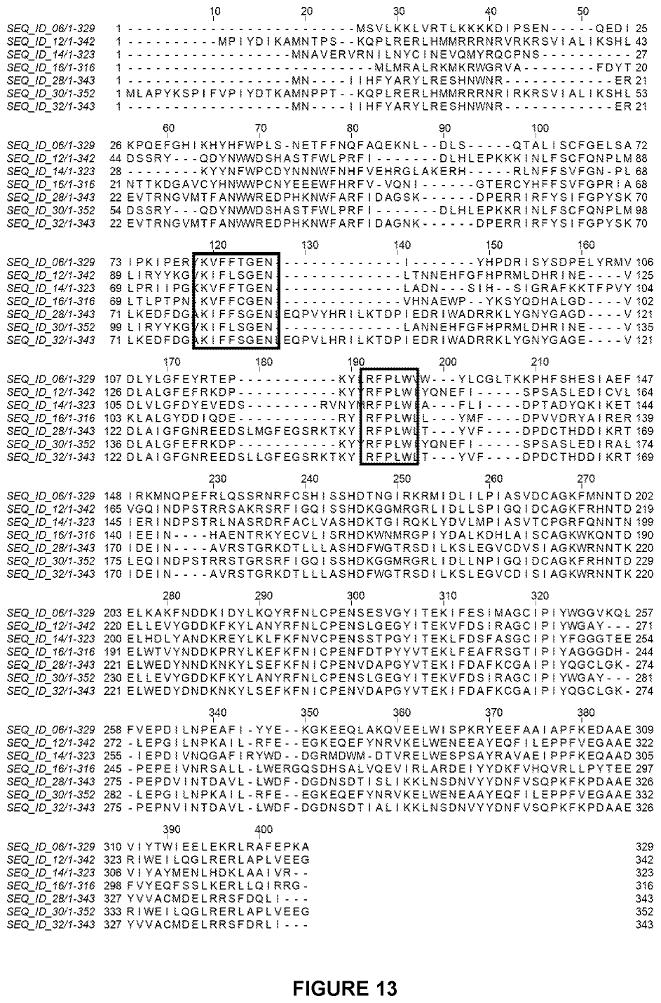

Mutant E. coli strains containing an expression construct for either SEQ ID NO: 4, SEQ ID NO: 20, SEQ ID NO: 22, SEQ ID NO: 24 and SEQ ID NO: 26 were evaluated for their 3-FL production in a growth experiment as described in Example 1. As indicated in FIG. 12, all polypeptide sequences contain the consensus domain DM[AS]VSF (SEQ ID NO: 36), but only SEQ ID NO: 2, SEQ ID NO: 4, SEQ ID NO: 20 and SEQ ID NO: 22 additionally contain the consensus motif [NH]XDPAXLD (SEQ ID NO: 35) in the N-terminal region of the protein. The strain containing the H. pylori alphα-1,3-fucosyltransferase fucT (SEQ ID NO: 18) was taken along as a positive control. All strains were grown in multiple wells of a 96-well plate and tested in standard medium with 30 g/L sucrose and 20 g/L lactose.

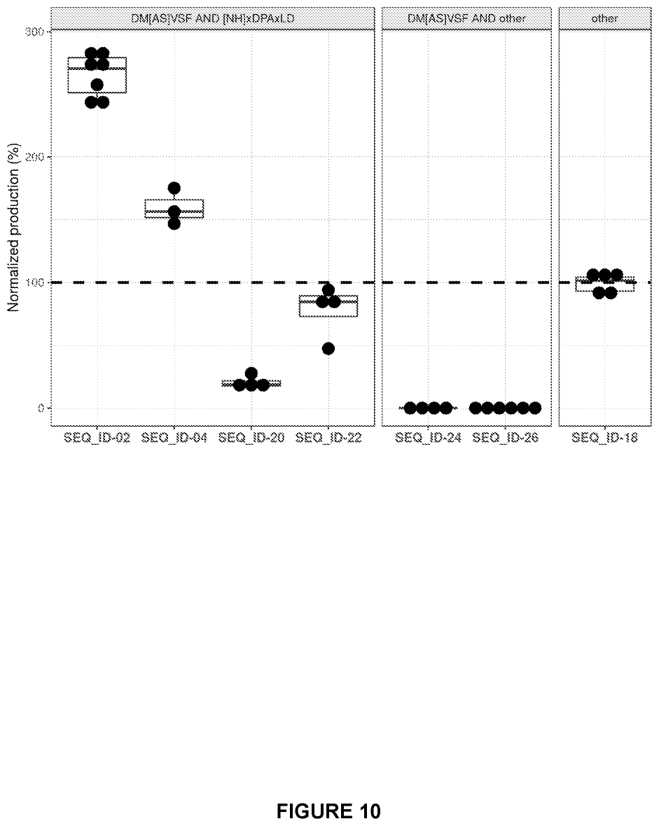

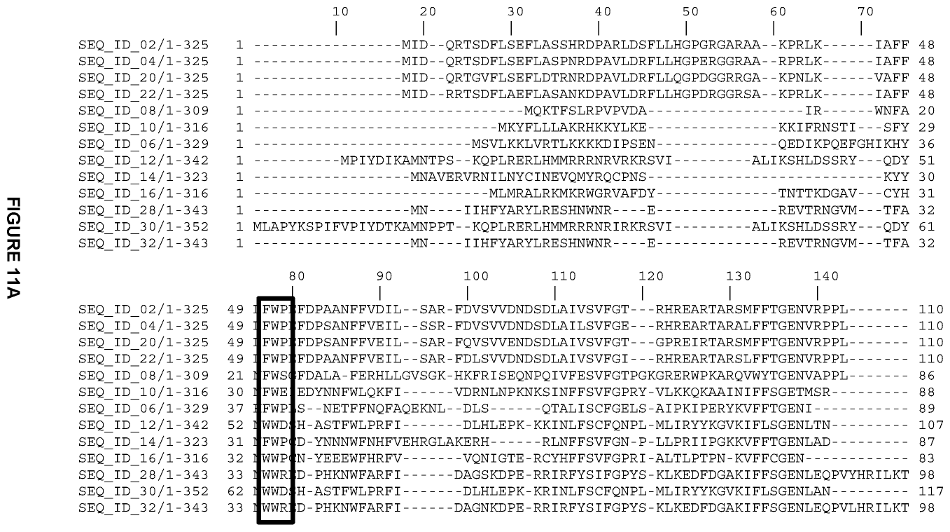

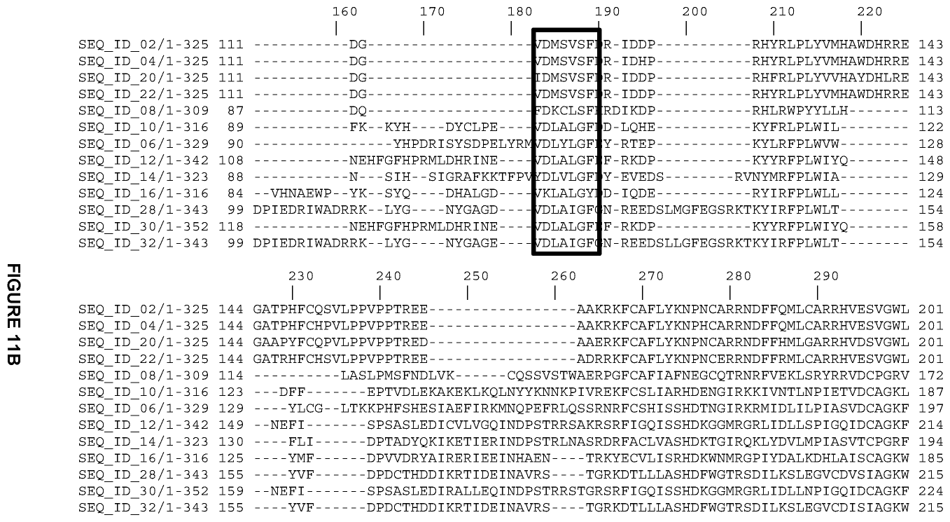

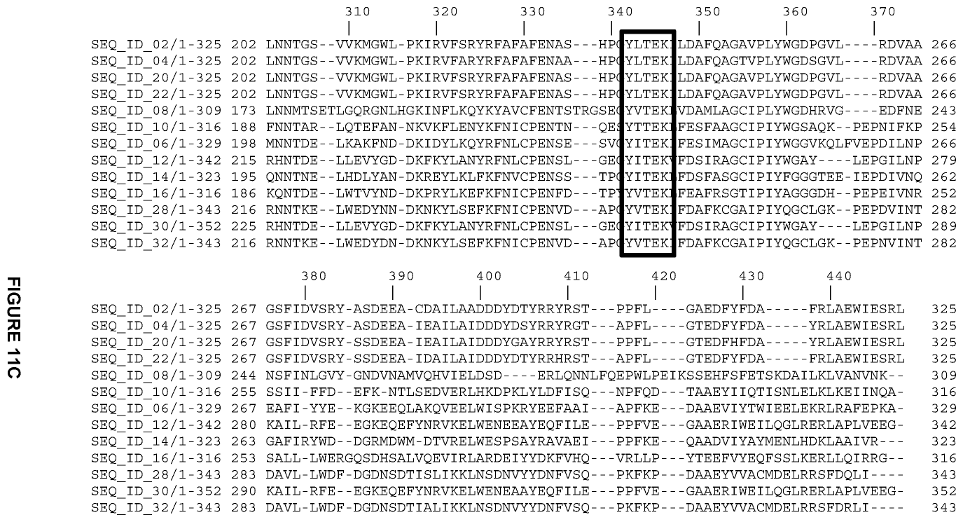

FIG. 10 shows the normalized production of 3-fucosyllactose produced by the strains. Each datapoint corresponds to data from one well. The dashed horizontal line indicates the setpoint to which all datapoints were normalized.

The experiment shows that only the strains containing polypeptides with both consensus motifs [NH]xDPAxLD and DM[AS]VSF: i.e., SEQ ID NO: 2, SEQ ID NO: 4, SEQ ID NO: 20 or SEQ ID NO: 22, are able to produce 3-FL, while the strains with polypeptides with DM[AS]VSF but lacking [NH]xDPAxLD: i.e., SEQ ID NO: 24 and SEQ ID NO: 26, do not produce any 3-FL. Based on this data, we can conclude that the presence of the [NH]xDPAxLD (SEQ ID NO: 35) consensus motif at the N-terminal region of polypeptides with the DM[AS]VSF (SEQ ID NO: 36) domain is crucial for the enzyme to have lactose binding alphα-1,3-fucosyltransferase activity.

Moreover, the polypeptide of SEQ ID NO: 22 has 92% global sequence identity to SEQ ID NO: 2, herewith showing that also sequences that have 87% or more sequence identity to SEQ ID NO: 2 have lactose binding alphα-1,3-fucosyltransferase activity.

### Example 15

### Evaluation of Strains Expressing Polypeptides with SEQ ID NO: 28, SEQ ID NO: 30 or SEQ ID NO: 32

Mutant E. colistrains containing an expression construct for either SEQ ID NO: 28, SEQ ID NO: 30 or SEQ ID NO: 32 can be evaluated for their 3-FL production in a growth experiment as described in Example 1. At the end of the growth experiment, the production of 3-fucosyllactose can be observed in the culture broth.

### Example 16

### Evaluation of the 3FL Purity at the End of a Fed-Batch Fermentation

Fed-batch fermentations at bioreactor scale were performed to evaluate strains, derived from the mutant E. coli K12 MG1655 strain background as described in Example 1, expressing various alphα-1,3-fucosyltransferase enzymes with SEQ ID NO: 2, SEQ ID NO: 6 and SEQ ID NO: 18. The bioreactor runs were performed as described in Example 1. In these examples, sucrose was used as a carbon source. Lactose was added in the batch medium at 90 g/L as a precursor for 3-FL formation, and a concentrated sucrose solution was fed during the fed-batch. For each strain, three independent fermentations were performed.

At the end of the fermentation, the broth was analyzed for the presence of lactose and 3-FL and the 3-FL purity was calculated using the formula 3FL (g/L)/(3FL (g/L)+lactose (g/L)). For strains containing SEQ ID NO: 18, an average purity of 85% was obtained, while for strains containing SEQ ID NO: 2 or 6 an average purity of over 98% and over 99% was obtained respectively.

The experiment shows that mutant E. coli strains expressing the lactose binding alphα-1,3-fucosyltransferase genes with SEQ ID NO: 2 or SEQ ID NO: 6 produce, in fed-batch fermentations at bioreactor scale, a broth with a higher 3-FL purity than similar strains containing SEQ ID NO: 18.

1.-39. (canceled)

40. A method of producing α-1,3-fucosyllactose, the method comprising:
contacting a polypeptide with a mixture comprising GDP-fucose as donor substrate, and lactose as acceptor substrate, under conditions wherein the polypeptide catalyzes the transfer of a fucose residue from the donor substrate to the acceptor substrate,
wherein the polypeptide has α-1,3-fucosyltransferase activity and is able to use lactose as acceptor substrate, wherein the polypeptide comprises:
i) an amino acid sequence comprising a conserved GDP-fucose binding domain [Y/W/L/H/F/M]X[T/S/C][E/Q/D/A][K/R] (SEQ ID NO: 33);
ii) an amino acid sequence comprising a conserved [K/D][L/K/M]XXX[F/Y] domain (SEQ ID NO: 34), and
iii) if ii) is DM[A/S]VSF (SEQ ID NO: 36), then a conserved motif [N/H]XDPAXLD (SEQ ID NO: 35) is present at the N-terminal region;
wherein X can be any distinct amino acid; and
wherein the C-terminus of the polypeptide has less than or equal to 100 amino acids starting from the first amino acid of the GDP-fucose binding domain;
so as to thereby produce α-1,3-fucosyllactose.

41. The method according to claim 40, wherein the polypeptide is provided in a cell-free system.

42. The method according to claim 40, wherein the polypeptide is produced by a cell comprising a polynucleotide encoding the polypeptide.

43. The method according to claim 42, wherein GDP-fucose and/or lactose is provided by a cell producing the GDP-fucose and/or lactose.

44. The method according to claim 42, wherein the cell is genetically modified to produce α-1,3-fucosyllactose, and wherein the cell comprises at least one polynucleotide encoding an enzyme for α-1,3-fucosyllactose synthesis, wherein the cell has the ability to use lactose as acceptor substrate.

45. The method according to claim 42,
wherein a cell is grown, which cell expresses the polypeptide with α-1,3-fucosvitransferase activity and with the ability to use lactose as acceptor substrate, under suitable nutrient conditions permissive for producing α-1,3-fucosyllactose, and also permissive for the expression of the polypeptide; and
wherein, simultaneously or subsequently thereto, a donor substrate GDP-fucose and the acceptor substrate lactose is provided, in order for the α-1,3-fucosyltransferase polypeptide to catalyze the transfer of a fucose residue from GDP-fucose to lactose, thereby producing α-1,3-fucosyllactose.

46. The method according to claim 42, wherein the cell is transformed or transfected to express an exogenous polypeptide with α-1,3-fucosyltransferase activity and with the ability to use lactose as an acceptor substrate.

47. The method according to claim 42, wherein the GDP-fucose and/or lactose is provided by an enzyme simultaneously expressed in the cell or by the metabolism of the cell.

48. The method according to claim 40, farther comprising purifying α-1,3-fucosyllactose.

49. The method according to claim 40, wherein the polypeptide is selected from the group consisting of:
i) any one of SEQ ID NOS: 2, 4, 6, 8, 10, 12, 14, 16, 20, 22, 28, 30, or 32;
ii) an amino acid sequence having 87% or more sequence identity to the full length amino acid sequence of SEQ ID NOS: 2, 20, or 22;
iii) an amino acid sequence having 80% or more sequence identity to the full length amino acid sequence of any one of SEQ ID NOS: 6, 8, 10, 12, 14, 16, 28, 30, or 32;
iv) a fragment of an amino acid sequence of SEQ ID NOS: 2, 20, or 22, wherein the fragment comprises at least 45 contiguous amino acids thereof; and
v) a fragment of an amino acid sequence of any one of SEQ ID NOS: 6, 8, 10, 12, 14, 16, 28, 30, or 32, wherein the fragment comprises at least 10 contiguous amino acids thereof and has lactose binding alphα-1,3-fucosyltransferase activity;
wherein, optionally, the polypeptide is modified by an N-terminal and/or C-terminal amino acid stretch.

50. The method according to claim 40, wherein the method comprises at least one of the following:
i) adding to a culture medium a lactose feed comprising at least 150 gram of lactose per initial reactor volume, in a continuous manner, so that the final volume of the culture medium is not more than two-fold of the volume of culture medium before addition of the lactose feed;
ii) adding a lactose feed in a continuous manner to the culture medium over the course of 1 day, 2 days, 3 days, 4 days, or 5 days by means of a feeding solution;
iii) adding a lactose feed in a continuous manner to the culture medium over the course of 1 day, 2 days, 3 days, 4 days, or 5 days by means of a feeding solution, wherein the concentration of the lactose feeding solution is 600 g/L, wherein the pH of the solution is set between 3 and 7 and wherein the temperature of the feed solution is kept between 20° C. and 80° C.; or
iv) wherein the method results in a 3-fucosyllactose concentration of at least 200 g/L in the final volume of the culture medium.

51. A cell comprising at least one polynucleotide encoding a polypeptide with α-1,3-fucosyltransferase activity and able to use lactose as acceptor substrate, wherein the polypeptide comprises:
i) an amino acid sequence comprising a conserved GDP-fucose binding domain [Y/W/L/H/F/M]X[T/S/C][E/Q/D/A][K/R] (SEQ ID NO: 33);
ii) an amino acid sequence comprising a conserved [K/D][L/K/M]XXX[F/Y] domain (SEQ ID NO: 34), and
iii) if the domain of ii) is DM[A/S]VSF (SEQ ID NO: 36), then a conserved motif [N/H]XDPAXLD (SEQ ID NO: 35) is present at the N-terminal region of the polypeptide;
wherein X can be any distinct amino acid; and
wherein the C-terminus of the polypeptide has less than or equal to 100 amino acids starting from the first amino acid of the GDP-fucose binding domain.

52. The cell of claim 51, whereinthe cell comprises:
i) a polynucleotide encoding the polypeptide with lactose binding α-1,3-fucosyltransferase activity, wherein the polynucleotide is foreign to the cell and wherein the polynucleotide is integrated into the cell's genome, or
ii) a vector comprising a polynucleotide encoding the polypeptide, wherein the polynucleotide is operably linked to control sequences recognized by a cell transformed with the vector.

53. The cell of claim 51, wherein the polypeptide comprises an amino acid sequence selected from the group consisting of:
i) any one of SEQ ID NOS: 2, 4, 6, 8, 10, 12, 14, 16, 20, 22, 28, 30, or 32;
ii) an amino acid sequence having 87% or more sequence identity to the full length amino acid sequence of SEQ ID NOS: 2, 20, or 22;
iii) an amino acid sequence having 80% or more sequence identity to the full length amino acid sequence of any one of SEQ ID NOS: 6, 8, 10, 12, 14, 16, 28, 30, or 32;
iv) a fragment of an amino acid sequence of SEQ ID NOS: 2, 20, or 22, wherein the fragment comprises at least 45 contiguous amino acids thereof; and
v) a fragment of an amino acid sequence of any one of SEQ ID NOS: 6, 8, 10, 12, 14, 16, 28, 30 or 32, wherein the fragment comprises at least 10 contiguous amino acids thereof and has lactose binding alphα-1,3-fucosyltransferase activity; and
wherein, optionally, the polypeptide is modified by an N-terminal and/or C-terminal amino acid stretch.

54. The method according to claim 42, wherein the cell is selected from the group consisting of a microorganism, plant cell, animal cell, bacterium, fungus, and yeast.

55. The cell of claim 51, wherein the cell is selected from the group consisting of a bacterium, an Escherichia coli strain, an Escherichia coli K12 strain, and Escherichia coli MG1655.

56. The cell of claim 51, wherein the cell is a yeast cell.

57. The cell of claim 51, wherein the polynucleotide is adapted to the codon usage of the respective cell.

58. A method of using the cell of claim 51 to produce α-1,3 fucosyllactose, the method comprising:
a) cultivating the cell in a medium under conditions permissive for producing α-1₂3-fucosyltransferase, and
b) optionally, separating the α-1,3-fucosyltransferase from the cultivation.

59. A microorganism that heterologously expresses a lactose binding α-1,3-fucosyltransferase polypeptide, wherein the polypeptide comprises:
i) an amino acid sequence comprising a conserved GDP-fucose binding domain [Y/W/L/H/F/M]X[T/S/C][E/Q/D/A][K/R] (SEQ ID NO: 33);
ii) an amino acid sequence comprising a conserved [K/D][L/K/M]XXX[F/Y] domain (SEQ ID NO: 34), and
iii) if ii) is DM[A/S]VSF (SEQ ID NO: 36), then a conserved motif [N/H]XDPAXLD (SEQ ID NO: 35) is present at the N-terminal region;
wherein X can be any distinct amino acid; and
wherein the C-terminus of the polypeptide has less than or equal to 100 amino acids starting from the first amino acid of the GDP-fucose binding domain.

60. The microorganism of claim 59, wherein the polypeptide comprises an amino acid sequence selected from the group consisting of:
i) any one of SEQ ID NOS: 2, 4, 6, 8, 10, 12, 14, 16, 20, 22, 28, 30, or 32;
ii) an amino acid sequence having 87% or more sequence identity to the full length amino acid sequence of SEQ ID NOS: 2, 20, or 22;
iii) an amino acid sequence having 80% or more sequence identity to the full length amino acid sequence of any one of SEQ ID NOS: 6, 8, 10, 12, 14, 16, 28, 30, or 32;
iv) a fragment of an amino acid sequence of SEQ ID NOS: 2, 20, or 22, wherein the fragment comprises at least 45 contiguous amino acids thereof;
v) a fragment of an amino acid sequence of any one of SEQ ID NOS: 6, 8, 10, 12, 14, 16, 28, 30, or 32, wherein the fragment comprises at least 10 contiguous amino acids thereof and has lactose binding alphα-1,3-fucosyltransferase activity; and
wherein, optionally, the polypeptide is modified by an N-terminal and/or C-terminal amino acid stretch.

61. A method of producing α-1,3-fucosyllactose, the method comprising:
utilizing the microorganism of claim 59 to produce alphα-1,3-fucosyllactose. 62, (New) The method according to claim 40, further comprising:
separating the α-1,3-fucosyllactose from a cell or a medium of its growth.

63. The method according to claim 62, wherein the separation comprises at least one of the following: clarification, ultrafiltration, nanofiltration, reverse osmosis, microfiltration, activated charcoal or carbon treatment, tangential flow high-performance filtration, tangential flow ultrafiltration, affinity chromatography, ion exchange chromatography, hydrophobic interaction chromatography and/or gel filtration, or ligand exchange chromatography.

64. The method according to claim 40, further comprising:
purifying α-1,3-fucosyllactose.

65. The method according to claim 64, wherein purifying α-1,3-fucosyllactose comprises at least one of the following: use of activated charcoal or carbon, use of charcoal, nanofiltration, ultrafiltration or ion exchange, use of alcohols, use of aqueous alcohol mixtures, crystallization, evaporation, precipitation, drying, spray drying, or lyophilization.

66. The method according to claim 42, wherein the polypeptide is produced in a cell selected from the group consisting of a fungal cell, yeast, bacterial, insect, animal, or plant expression system 67, (New) The method according to claim 66, wherein the cell is a bacterium, Escherichia coli, an Escherichia coli K12 strain, or Escherichia coli MG1655.

68. The method according to claim 66, wherein the cell is a yeast cell.

69. The method according to claim 40, wherein the lactose concentration in culture medium ranges from 50 to 150 g/L.

70. The method according to claim 40, wherein the final concentration of 3-fucosyllactose ranges between 70 g/L to 200 g/L.

71. The method according to claim 40, wherein the production results in a lactose concentration to 3-fucosyllactose concentration ratio of less than 1:5 at the end of fermentation.

72. The method according to claim 40, wherein the 3-fucosyllactose thus produced has a purity of 80% or more.

73. The method according to claim 40, wherein the catalysis results in a lactose concentration to 3-fucosyllactose concentration ratio of less than 1:5 at the end of fermentation.

74. The method according to claim 40, wherein the catalysis results in a 3-fucosyllactose purity of 80% or more at the end of fermentation.

75. The method according to claim 50, resulting in a 3-fucosyllactose purity of 80% or more in the final volume of the culture.
